SEQUENCE LISTING (3 sequences)

SEQ ID NO: 1 - protein, 109 aa, synthetic construct
  REGION 1..109: note = Synthetic Construct
SLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVT

SEQ ID NO: 2 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 3 - protein, 211 aa, Homo sapiens [sequence omitted]

(19) United States
(12) Patent Application Publication
(10) Pub. No.: US 2026/0157951 A1
(43) Pub. Date: Jun. 11, 2026

(54) METHODS AND COMPOSITIONS FOR HAIR FOLLICLE REGENERATION AND HAIR GROWTH

(71) Applicant: Samuel E. Lynch, Franklin, TN (US)

(72) Inventor: Samuel E. Lynch, Franklin, TN (US)

(21) Appl. No.: 19/284,522

(22) Filed: Jul. 29, 2025

Related U.S. Application Data
(63) Continuation-in-part of application No. 18/752,711, filed on Jun. 24, 2024.
(60) Provisional application No. 63/522,989, filed on Jun. 23, 2023.
(60) Provisional application No. 63/523,584, filed on Jun. 27, 2023.
(60) Provisional application No. 63/676,605, filed on Jul. 29, 2024.

(51) Int. Cl. A61K 8/64; A61K 8/73; A61K 38/18; A61P 17/14; A61Q 7/00; A61Q 19/08

(52) U.S. Cl. CPC A61K 8/64; A61K 8/735; A61K 38/1858; A61P 17/14; A61Q 7/00; A61Q 19/08; A61K 2800/91

(57) ABSTRACT

Compositions of PDGF for skin or hair follicle repair, regeneration, rejuvenation, comfort, reinforcement or aesthetic utility and hair and growth comprising rhPDGF-BB and/or rhPDGF-AB combined with a carrier selected from the group consisting of differing types of collagen, gelatin, alginates, polysaccharides, glycosaminoglycans, fibrin, hyaluronic acid and derivatives or combinations thereof, wherein skin, hair follicles and surrounding skin, and/or an area of hair loss is treated by applying the disclosed compositions.

### PRIORITY CLAIM

This present application is a Continuation-in-part of currently pending U.S. patent application Ser. No. 18/752,711, filed Jun. 24, 2024 which claims priority to U.S. Provisional Patent Application No. 63/523,584 filed on Jun. 27, 2023 and U.S. Provisional Patent Application No. 63/522,989 filed on Jun. 23, 2023, and in addition, claims priority to U.S. Provisional Patent Application No. 63/676,605 filed on Jul. 29, 2024, the contents of which all applications are incorporated herein by reference in their entireties.

### FIELD OF THE INVENTION

The present invention relates to methods and compositions for using recombinant human platelet-derived growth factor (rhPDGF) for hair follicle regeneration and hair growth and for repair, rejuvenation, reinforcement, and regeneration of skin and soft tissues and to improve comfort and aesthetics thereof.

### DESCRIPTION OF THE TEXT FILE SUBMITTED ELECTRONICALLY

The contents of the text file submitted electronically herewith are incorporated herein by reference in their entirety: A computer readable format copy of the Sequence Listing titled: 133996_PCT_25_July_2025.xml, date recorded: 25 Jul. 2025, file size 4,364 bytes.

### BACKGROUND OF THE INVENTION

A well-known demand exists for cosmetic procedures to repair and restore disruptions of the skin and hair follicles to stimulate hair growth. The hair follicle is a complex skin appendage that undergoes lifelong cyclical remodeling, but, in humans, no additional hair follicles are formed after birth. When hair growth machinery is compromised, hair follicles cannot regenerate on their own. Illness, hormonal changes, stress, aging, and genetic conditions can interfere with the hair's growth cycle and cause hair loss that doesn't grow back on both the head and body. Baldness, the most common type of hair loss, affects an estimated 80 million individuals in the US alone. Not only does hair loss require additional protection of the scalp, but it can be an emotionally distressing issue that causes unpleasing appearance and lack of self-confidence in many individuals. Numerous treatments, from medications to hair transplants exist, with the medications having a limited and unpredictable affect. Thus, a large demand remains for minimally invasive procedures that effectively stimulates and activates hair growth with minimal to no pain and a quick recovery time.

Platelet-derived growth factor (PDGF) is a family of disulfide-bonded dimeric molecules of A-, B-, C- and D-polypeptide chains (PDGF-AA, -AB, -BB, -CC and -DD). The four PDGFs are inactive in their monomeric forms. The PDGFs bind to the protein tyrosine kinase receptors PDGF receptor-α and -β. These two receptor isoforms dimerize upon binding the PDGF dimer, leading to three possible receptor combinations, namely -αα, -ββ and -αβ. The extracellular region of the receptor consists of five immunoglobulin-like domains while the intracellular part is a tyrosine kinase domain. The ligand-binding sites of the receptors are located to the three first immunoglobulin-like domains. PDGF-AB and PDGF-CC specifically interact with PDGFR-αα and -αβ, but not with -ββ. PDGF-DD binds to PDGFR-ββ with high affinity, and to PDGFR-αβ to a markedly lower extent and is therefore regarded as PDGFR-ββ specific. PDGF-AA binds only to PDGFR-αα, while PDGF-BB is the only PDGF that can bind all three receptor combinations with high affinity.

rhPDGF-BB is known in art for use in bone regeneration and augmentation for both orthopedic and periodontal bone indications, and more recently novel methods and compositions to treat wounds, such as ulcers and burns have been discovered. The cellular and molecular mechanisms involved in bone regeneration versus wound healing are very complex, radically different and in many cases in direct conflict. The differing processes involve the formation of radically different extracellular matrices, mineralization versus no mineralization, different cell signaling and different cell types. Furthermore, the repair environments are very different, including vascularity for blood supply, cellular protein production involved in the repair process, different innervation, and different biomechanics (i.e., skin elasticity versus bone rigidity). rhPDGF-AB is known in the art for its role in post-myocardial infarction cardiac wound repair, showing promise in improving cardiac function and reducing ventricular arrhythmias by facilitating more organized scar formation. Additionally, rhPDGF-AB enhances angiogenesis and supports the formation of new blood vessels. Therefore, the PDGF composition, dosage ranges and treatment protocols known to treat bone repair inside the body had to be adapted to treat wounds.

It is generally expected that these known uses of PDGF delivered to bone inside the body or to non-intact skin would be ineffective and even cause worsening of aesthetics. For example, the methods and compositions of PDGF known in the art stimulate osteoblast proliferation and bone formation could lead to calcification of the skin, or fibroblast growth and collagen production, which potentially leads to better healing of wounds, also has the potential to cause hypertrophic scarring leading to terrible complications and disfigurations. Furthermore, PDGF stimulates angiogenesis, i.e., new blood vessel formation, and increased blood vessels in the face could be a serious disfiguring side effect of the use of PDGF in cosmetic procedures. Thus, the current methods, including the delivery systems, and therapeutic compositions of PDGF to treat bone and tissue inside the body would not be used by one skilled in the art to promote dermal and hair follicle repair and rejuvenation.

However, given the recent developments to treat different types of wounds using PDGF based on variations in composition and treatment regimens, a demand exists for an effective use of PDGF to improve, repair, regenerate or rejuvenate cosmetic and other disruptions to the hair follicles and surrounding dermal or subdermal layers of the skin in a patient-friendly and consistent manner.

### SUMMARY OF THE INVENTION

The present disclosure describes compositions of PDGF and corresponding methods for improving skin and hair repair and growth and to aid in the regeneration and rejuvenation of the skin and hair follicles by using the disclosed compositions. The present embodiments leverage the attributes of rhPDGF-BB and/or PDGF-AB for the cosmetic treatment of hair loss and/or promote hair growth (i.e., to improve one or more of total hair count, hair density, mean thickness, and cumulative hair thickness) for any reason, such as hair loss that arises from stress, hormonal imbalance, disease, infection, and nutritional deficiencies. The disclosed compositions of rhPDGF-BB and/or PDGF-AB may be used for improving tissue integrity and reinforcement, promoting hair growth, thickness, and strength, and for aesthetic utility in cosmetics and cosmeceuticals or to improve comfort and reduce pain, inflammation, redness, and swelling resulting from disruptions of the skin and hair follicles.

In an embodiment, an rhPDGF-BB and/or rhPDGF-AB-based composition for treating disrupted or diseased skin, hair follicles and surrounding skin, and/or an area of hair loss includes a composition of rhPDGF-BB and/or rhPDGF-AB and a biocompatible carrier selected from the group consisting of differing types of collagen, gelatin, alginates, polysaccharides, glycosaminoglycans, fibrin and derivatives or combinations thereof. In some embodiments, the composition comprises rhPDGF-BB. In some embodiments, the composition comprises rhPDGF-AB. In some embodiments, the composition comprises rhPDGF-BB and rhPDGF-AB. Preferably, the composition comprises rhPDGF-BB and hyaluronic acid as the carrier. In some embodiments, the composition comprises rhPDGF-AB and hyaluronic acid as the carrier. In some embodiments, the composition comprises rhPDGF-BB and rhPDGF-AB and hyaluronic acid as the carrier. In another embodiment, an rhPDGF-BB and rhPDGF-AB-based composition for treating the disrupted or diseased skin, hair follicles and surrounding skin, and/or an area of hair loss includes a composition of rhPDGF-BB and/or rhPDGF-AB only in a physiologic solution (i.e., without a carrier). In another embodiment, an rhPDGF-BB-based composition for treating the disrupted or diseased skin, hair follicles and surrounding skin, and/or an area of hair loss includes a composition of rhPDGF-BB only (i.e., without a carrier). In another embodiment, an rhPDGF-AB-based composition for treating the disrupted or diseased skin, hair follicles and surrounding skin, and/or an area of hair loss includes a composition of rhPDGF-AB only (i.e., without a carrier). A person skilled in the art would understand that the disclosed compositions may include any combination of the above-mentioned ingredients and are not limited to the above-mentioned compositions only.

The present disclosure also describes methods of treating skin, hair follicles and surrounding skin, and/or an area of hair loss by applying any of the disclosed compositions. In one aspect, the invention provides a cosmetic method of treating a skin disruption on a subject, wherein the skin disruption does not involve tendons, ligaments, or bones, wherein said method comprises: (i) providing a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier, wherein the biocompatible carrier is a hyaluronic acid, collagen, gelatin, alginate, polysaccharide, glycosaminoglycan, fibrin, a derivative thereof or a combination thereof; and (ii) applying the composition onto or into the skin disruption, wherein the carrier delivers biologically active rhPDGF onto or into the skin disruption leading to regeneration, rejuvenation or repair of the skin disruption. In one aspect, the invention provides a cosmetic method of promoting hair follicle regeneration and/or hair growth in a subject, wherein said method comprises: (i) providing a composition comprising or consisting essentially of recombinant human PDGF-BB (rhPDGF-BB) and/or recombinant human PDGF-AB (rhPDGF-AB) in a physiologic solution; and (ii) applying the composition onto or into the hair follicles and surrounding skin and/or an area of hair loss. In a further aspect, the invention provides a method of reducing or improving the pain or discomfort caused by a skin disruption on a subject, wherein the disruption does not involve tendons, ligaments, or bones, wherein said method comprises: (i) providing a composition comprising or consisting essentially of sterile recombinant human PDGF-BB (rhPDGF-BB) and/or sterile recombinant human PDGF-AB (rhPDGF-AB) in a physiologic solution, and optionally further comprising a sterile biocompatible carrier that is a hyaluronic acid, collagen, gelatin, alginate, polysaccharide, glycosaminoglycan, fibrin, a derivative thereof or a combination thereof; and (ii) applying the composition onto or into the skin disruption leading to reduced pain or discomfort caused by the skin disruption.

In some embodiments, the composition comprises hyaluronic acid as the carrier. In an illustrative embodiment, the method includes mixing 1 to 5 milliliters (mL) of a solution containing hyaluronic acid (a linear polysaccharide also known as hyaluronan or hyaluronate) with 0.5 mL to 5 mL of rhPDGF-BB solution in a sodium acetate buffer having a pH value between 5.5-6.5 to form a composition; and applying the composition to a surface of the disrupted or diseased skin, hair follicles and the surrounding skin, and/or an area of hair loss. In some embodiments, the composition is applied to an area of hair loss. In a further illustrative embodiment, the method includes mixing 1 to 10 milliliters (mL) of a solution containing hyaluronic acid (a linear polysaccharide also known as hyaluronan or hyaluronate) with 0.5 mL to 5 mL of rhPDGF-AB solution in a sodium acetate buffer having a pH value between 5.5-6.5 to form a composition; applying the composition to a surface of the disrupted or diseased skin, hair follicle and surrounding skin, and/or area of hair loss. In a further illustrative embodiment, the method includes mixing 1 to 5 milliliters (mL) of a solution containing hyaluronic acid (a linear polysaccharide also known as hyaluronan or hyaluronate) with 0.5 mL to 5 mL of rhPDGF-BB and rhPDGF-AB solution in a sodium acetate buffer having a pH value between 5.5-6.5 to form a composition; applying the composition to a surface of the disrupted or diseased skin, hair follicle and surrounding skin, and/or area of hair loss.

In some embodiments, the composition is applied to a surface of the disrupted or diseased skin. In some embodiments, the composition is applied to hair follicles and the surrounding skin. In some embodiments, the composition is applied to an area of hair loss. The composition may be applied to intact, nondeficited but aged skin, hair follicles and surrounding skin, and/or an area of hair loss, or to skin, hair follicles and surrounding skin, and/or an area of hair loss disrupted by accidental or intentional injury into the dermal or subdermal layers of the skin. In some embodiments, the skin disruption is on healthy skin. In some embodiments, the skin disruption is caused by aging. In some embodiments, the skin disruption is caused by inflammation, radiation damage, ultraviolet damage, or combinations thereof. In some embodiments, the skin disruption is caused by an incision from a surgical procedure or other medical intervention, such as chemotherapy, radiation, or medication. In some embodiments, the skin disruption is a scar. In some embodiments, the skin disruption is caused by an aesthetic procedure, as described herein, such as micro-needling, micro-injections, micro-coring, dermaplaning, dermal abrasion, dermal filling or laser resurfacing. In some embodiments, the hair follicles and surrounding skin are healthy. In some embodiments, the hair follicles and surrounding skin are on a subject having a reduction in one or more of total hair count, hair density, mean thickness, and/or cumulative hair thickness, in comparison to measurements taken from the subject at a previous time or average measurements. For example, the subject may have a reduction in any two, three, or four of total hair count, hair density, mean thickness, and/or cumulative hair thickness, in comparison to measurements taken from the subject at a previous time or average measurements. In some embodiments, the reduced total hair count, reduced hair density, reduced mean thickness, and/or reduced cumulative hair thickness is caused by aging. In some embodiments, the reduced total hair count, hair density, mean thickness, and/or cumulative hair thickness is caused by inflammation, radiation damage, ultraviolet damage, or combinations thereof. In some embodiments, the reduced total hair count, hair density, mean thickness, and/or cumulative hair thickness is caused by an incision from a surgical procedure or other medical intervention, such as chemotherapy, radiation, or medication. In some embodiments, the reduced total hair count, hair density, mean thickness, and/or cumulative hair thickness is caused by scarring. In some embodiments, the reduced total hair count, hair density, mean thickness, and/or cumulative hair thickness is caused by an aesthetic procedure, as described herein, such as micro-needling, micro-injections, micro-coring, dermaplaning, dermal abrasion, dermal filling or laser resurfacing. In some embodiments, the area of hair loss is on healthy skin. In some embodiments, the area of hair loss is caused by ageing. In some embodiments, the area of hair loss is caused by inflammation, radiation damage, ultraviolet damage, or combinations thereof. In some embodiments, the area of hair loss is caused by an incision from a surgical procedure or other medical intervention, such as chemotherapy, radiation, or medication. In some embodiments, the area of hair loss is due to a scar. In some embodiments, the area of hair loss is caused by an aesthetic procedure, as described herein, such as micro-needling, micro-injections, micro-coring, dermaplaning, dermal abrasion, dermal filling or laser resurfacing.

In aesthetic procedures, including those to stimulate hair growth, the compositions disclosed herein can be applied to a skin or other disruption as a standalone method or before, during, and/or after other aesthetic procedures, as further described herein. In aesthetic procedures to promote hair follicle regeneration and/or stimulate hair growth, the compositions disclosed herein can be applied onto or into hair follicles and the surrounding skin or an area of hair loss as a standalone method or before, during, and/or after other aesthetic procedures, as further described herein. In some embodiments, the composition may be applied topically to a skin or other disruption, and may be applied before, during and/or after a micro-needling, micro-injections, micro-coring, dermal abrasion, dermaplaning, dermal filling, laser resurfacing procedure, or other non-invasive skin resurfacing procedure. In some embodiments, the composition may be applied topically to hair follicles and the surrounding skin or an area of hair loss. For example, the composition may be applied topically before, during and/or after a micro-needling, micro-injections, micro-coring, dermal abrasion, dermaplaning, dermal filling, laser resurfacing procedure, or other non-invasive skin resurfacing procedure. In some embodiments, the composition may be applied by injection to hair follicles and the surrounding skin or an area of hair loss. For example, the composition may be applied by injection before, during and/or after a micro-needling, micro-injections, micro-coring, dermal abrasion, dermaplaning, dermal filling, laser resurfacing procedure, or other non-invasive skin resurfacing procedure. In some embodiments, the injection is sub-dermal injection. In some embodiments, the composition may be applied by micro-needling, laser or other penetration of the skin. In some embodiments, the composition may be applied by micro-injection. In some embodiments, the composition may be applied by microchannel microinjector.

The above-described procedure may be repeated periodically, for example weekly, every few weeks, monthly or every few months for up to about six times, followed by a healing period of varying lengths depending on clinical requirements and then repeated.

The inventive embodiments provided in this Summary of the Invention are meant to be illustrative only and to provide an overview of selected embodiments disclosed herein. The Summary of the Invention, being illustrative and selective, does not limit the scope of any claim, does not provide the entire scope of inventive embodiments disclosed or contemplated herein, and should not be construed as limiting or constraining the scope of this disclosure or any claimed inventive embodiment.

### BRIEF DESCRIPTION OF THE DRAWINGS

FIG. 1 shows a photographic image of a patient taken prior to treatment with PDGF Example Composition 1.

FIG. 2 shows a photographic image of a patient taken after one treatment with PDGF Example Composition 1. The image is taken two weeks after the first treatment.

FIG. 3 shows a photographic image of a patient taken after two treatments with PDGF Example Composition 1. The image is taken 4 and a half months (19 weeks and 2 days) after the first treatment.

FIG. 4 shows photographic images of a patient before and after two treatments with a PDGF composition. FIG. 4A shows an image of the patient taken prior to treatment. FIG. 4B shows an image of the patient taken 1 month after the second treatment, which was administered 4-8 weeks after the first.

FIG. 5 shows photographic images of a patient taken before and after two treatments with a PDGF composition. The second image was taken 1 month after the second treatment, which was administered 4-8 weeks after the first.

FIG. 6 shows photographic images of a patient taken before and after two treatments with a PDGF composition. The second image was taken 1 month after the second treatment, which was administered 4-8 weeks after the first.

FIG. 7 shows photographic images of a patient taken before and after two treatments with a PDGF composition. The second image was taken 1 month after the second treatment, which was administered 4-8 weeks after the first.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown but is to be accorded the widest possible scope consistent with the principles and features disclosed herein.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Certain terms and phrases have been used throughout the disclosure and will have the following meanings in the context of the ongoing disclosure.

As used herein, the article “a” means one or more unless explicitly stated otherwise.

The meaning of abbreviations is as follows: “C” means Celsius or degrees Celsius, as is clear from its usage, “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “g” means gram(s), “μg” or “ug” means microgram(s), “ng” means nanogram(s), “% w/v” means weight/volume percent, and “wt %” means percentage by weight.

The terms “comprising,” “contain,” “containing,” “including,” and “having,” as used in the claim and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition, or step being referred to is an optional (not required) feature of the invention.

As used herein the term “consisting essentially of” refers to those elements required for a given embodiment. The term permits the presence of additional elements that do not materially affect the basic and novel or functional characteristic(s) of that embodiment of the invention.

Some embodiments herein contemplate numerical ranges. When a numerical range is provided, the range includes the range endpoints unless otherwise indicated. Unless otherwise indicated, numerical ranges include all values and subranges therein as if explicitly written out.

Some values herein are modified by the term “about.” In some instances, the term “about” in relation to a reference numerical value can include a range of values plus or minus 10% from that value. For example, the amount “about 10” can include amounts from 9 to 11. In other embodiments, the term “about” in relation to a reference numerical value can include a range of values plus or minus 10%, 9%, 8%, 7%, 6%, 5%, 4%, 3%, 2%, or 1% from that value.

### Compositions

As described herein, it has been observed that recombinant human platelet-derived growth factor BB (rhPDGF-BB) effectively accelerates repair or rejuvenation of skin and hair follicles, such as improved appearance of the skin or tissues, or improves the quality or integrity of tissues such as skin and facilitates and restores hair growth, by causing an immediate influx of regenerative cells via a process called chemotaxis. Further, rhPDGF-BB also stimulates the proliferation or growth of regenerative cells thereby via a process called mitogenesis. In a third mechanism, rhPDGF-BB induces new capillaries and blood vessels to grow and become stable enhancing the tissue environment for healthy tissue growth or rejuvenation or regeneration. Moreover, the beta chain in recombinant human platelet-derived growth factor AB (rhPDGF-AB) is the same beta chain present in rhPDGF-BB. Therefore, the inventors have discovered that rhPDGF-AB also acts via these three mechanisms to function similarly to the PDGF-BB, to cause an immediate influx of regenerative cells, to stimulate the proliferation or growth of regenerative cells, and to induce new capillaries and blood vessels to grow and become stable. Compositions comprising rhPDGF-BB and/or rhPDGF-AB are therefore useful for treatment of skin, hair follicles and surrounding skin, areas of hair loss, tissue disruptions such as caused by injury or trauma, and disease processes, and deficits in the healthy integumental integrity such as those that result from aging. Further, compositions of rhPDGF-BB and/or rhPDGF-AB are useful for improving tissue integrity and reinforcement, for stimulating hair regrowth, or to improve comfort and reduce pain, inflammation, redness, and swelling resulting from disruptions of the skin and hair follicles.

As used herein, the terms “recombinant human platelet-derived growth factor BB” and “rhPDGF-BB” mean recombinant human platelet-derived growth factor BB and any derivative or isoform of PDGF (i.e., PDGF-AA, -AB, -BB, -CC, and -DD) that binds to and induces dimerization of the αα-, ββ- or αβ-receptor (PDGFR-αα, PDGFR-ββ or PDGFR-αβ), including peptides, small molecules, or mimetics containing a receptor binding domain that binds to PDGFR-αα, PDGFR-ββ or PDGFR-αβ.

As used herein, the terms “recombinant human platelet-derived growth factor AB” and “rhPDGF-AB” mean recombinant human platelet-derived growth factor AB and any derivative or isoform of PDGF (i.e., PDGF-AA, -AB, -BB, -CC, and -DD) that binds to and induces dimerization of the αα- or αβ-receptor (PDGFR-αα or PDGFR-αβ), including peptides, small molecules, or mimetics containing a receptor binding domain that binds to PDGFR-αα or PDGFR-αβ.

In one aspect, the present invention provides a composition comprising recombinant human platelet-derived growth factor BB (rhPDGF-BB) and/or recombinant human platelet-derived growth factor AB (rhPDGF-AB). In some embodiments, the composition comprises rhPDGF-BB and rhPDGF-AB. In some embodiments, the composition comprises rhPDGF-BB. In some embodiments, the composition comprises rhPDGF-AB. In some embodiments, the composition comprises rhPDGF-BB or a derivative or isoform of PDGF (i.e., PDGF-AA, -AB, -BB, -CC, and -DD), a peptide, a small molecule, or a mimetic containing a receptor binding domain that binds to and induces dimerization of the αα-, ββ- or αβ-receptor (PDGFR-αα, PDGFR-ββ or PDGFR-αβ); and/or rhPDGF-AB or a derivative or isoform of PDGF (i.e., PDGF-AA, -AB, -BB, -CC, and -DD), a peptide, a small molecule, or a mimetic containing a receptor binding domain that binds to and induces dimerization of the αα- or αβ-receptor (PDGFR-αα or PDGFR-αβ). In some embodiments, the composition comprises rhPDGF-BB or a derivative or isoform of PDGF (i.e., PDGF-AA, -AB, -BB, -CC, and -DD), a peptide, a small molecule, or a mimetic containing a receptor binding domain that binds to and induces dimerization of the αα-, ββ- or αβ-receptor (PDGFR-αα, PDGFR-ββ or PDGFR-αβ). In some embodiments, the composition comprises rhPDGF-AB or a derivative or isoform of PDGF (i.e., PDGF-AA, -AB, -BB, -CC, and -DD), a peptide, a small molecule, or a mimetic containing a receptor binding domain that binds to and induces dimerization of the αα- or αβ-receptor (PDGFR-αα or PDGFR-αβ). In some embodiments, the composition comprises rhPDGF-BB or a derivative or isoform of PDGF (i.e., PDGF-AA, -AB, -BB, -CC, and -DD), a peptide, a small molecule, or a mimetic containing a receptor binding domain that binds to and induces dimerization of the αα-, ββ- or αβ-receptor (PDGFR-αα, PDGFR-ββ or PDGFR-αβ); and rhPDGF-AB or a derivative or isoform of PDGF (i.e., PDGF-AA, -AB, -BB, -CC, and -DD), a peptide, a small molecule, or a mimetic containing a receptor binding domain that binds to and induces dimerization of the αα- or αβ-receptor (PDGFR-αα or PDGFR-αβ).

In some embodiments, the composition comprises sterile rhPDGF-BB and/or rhPDGF-AB in a physiologic solution. In some embodiments, the composition comprises sterile rhPDGF-BB and rhPDGF-AB in a physiologic solution. In some embodiments, the composition comprises sterile rhPDGF-BB in a physiologic solution. In some embodiments, the composition comprises sterile rhPDGF-AB in a physiologic solution. In some embodiments, the composition does not comprise growth factors other than PDGF. In some embodiments, the composition is useful for promoting skin and hair follicle regeneration, rejuvenation and repair. For example, the composition may be useful for treating a skin disruption in a subject. In some embodiments, the composition is useful for preventing or reducing pain or discomfort caused by a skin disruption in a subject. In some embodiments, the composition is useful for improving pain or discomfort caused by a skin disruption in a subject. In some embodiments, the composition may be useful for preventing or reducing redness, swelling and/or inflammation caused by a skin disruption. In some embodiments, the composition may be useful for activating and promoting hair regrowth. In some embodiments, the composition is useful for promoting hair follicle regeneration. In some embodiments, the composition is useful for promoting hair growth (i.e., improving one or more of total hair count, hair density, mean thickness, and cumulative hair thickness). As used throughout the disclosure, the term “composition” may be used interchangeably with the terms “therapeutic composition” or “cosmetic composition”.

In some embodiments, the composition (e.g. therapeutic or cosmetic) comprises rhPDGF-BB and/or rhPDGF-AB and a biocompatible carrier. In some embodiments, the composition comprises rhPDGF-BB and rhPDGF-AB and a biocompatible carrier. In some embodiments, the composition comprises rhPDGF-BB and a biocompatible carrier. In some embodiments, the composition comprises rhPDGF-AB and a biocompatible carrier. In some embodiments the composition is a therapeutic composition. In some embodiments, the composition is a cosmetic composition. In some embodiments, the composition comprises sterile recombinant human PDGF-BB (rhPDGF-BB) and/or sterile recombinant human PDGF-AB (rhPDGF-AB) in a physiologic solution and a sterile biocompatible carrier. In some embodiments, the composition comprises sterile rhPDGF-BB and sterile rhPDGF-AB in a physiologic solution and a sterile biocompatible carrier. In some embodiments, the composition comprises sterile rhPDGF-BB in a physiologic solution and a sterile biocompatible carrier. In some embodiments, the composition comprises sterile rhPDGF-AB in a physiologic solution and a sterile biocompatible carrier. The “composition” of rhPDGF-BB and/or rhPDGF-AB may be used for application on skin or connective tissues, either intact or damaged, and may include a therapeutic or cosmetic composition of rhPDGF-BB and/or rhPDGF-AB and a carrier. Compositions comprising rhPDGF-BB and/or rhPDGF-AB and a carrier are especially useful for promoting skin regeneration, rejuvenation and repair e.g. in a subject that has a disruption. In some embodiments, compositions comprising rhPDGF-BB and/or rhPDGF-AB and a carrier are also especially useful for promoting hair follicle regeneration. In some embodiments, compositions comprising rhPDGF-BB and/or rhPDGF-AB and a carrier are also especially useful for promoting hair growth (i.e., improving one or more of total hair count, hair density, mean thickness, and cumulative hair thickness). In some embodiments, compositions comprising rhPDGF-BB and/or rhPDGF-AB and a carrier are used for cosmetic and non-therapeutic purposes. As used herein, the term “carrier” is intended to refer broadly to any biologically compatible substance that can serve as a delivery vehicle for PDGF, whereas the term “matrix” is to refer to a carrier that acts as a substrate for cell attachment and/or vascular ingrowth, and/or provides a means for trapping the PDGF within its structure (such as, for example, through interconnected pores), thereby allowing for an ongoing or delayed or prolonged delivery of PDGF as a disruption heals. The biocompatible carrier may be sterile and/or porous. Preferably, the biocompatible carrier is sterile. In some embodiments, the carrier is non-porous. The term “non-porous” means that the carrier does not comprise interconnected pores that can trap PDGF within its structure.

In some embodiments, the composition (e.g. therapeutic or cosmetic) comprises only the rhPDGF-BB and/or rhPDGF-AB in a buffer solution without a carrier. In some embodiments, the composition comprises only the rhPDGF-BB and rhPDGF-AB in a buffer solution without a carrier. In some embodiments, the composition comprises only the rhPDGF-BB in a buffer solution without a carrier. In some embodiments, the composition comprises only the rhPDGF-AB in a buffer solution without a carrier. In some embodiments the composition is a therapeutic composition. In some embodiments, the composition is a cosmetic composition. In some embodiments, the rhPDGF-BB and/or rhPDGF-AB are sterile. As described herein, such compositions may be useful for treating a skin disruption, or for promoting repair, regenerative and rejuvenation of a skin disruption in a subject. As described herein, such compositions are useful for preventing or reducing or improving pain or discomfort at the site of a skin disruption in a subject. Such compositions may also be useful for preventing or reducing swelling, redness, and/or inflammation at the site of a skin disruption in a subject. Such compositions may also be useful for preventing or reducing scarring at the site of a skin disruption in a subject. Such compositions may also be useful for stimulating hair regrowth in a subject. Such compositions may also be useful for promoting hair follicle regeneration. Such compositions may also be useful for promoting hair growth (i.e., improving one or more of total hair count, hair density, mean thickness, and cumulative hair thickness). In some embodiments, when the composition comprises PDGF (rhPDGF-BB and/or rhPDGF-AB) without a carrier, the composition may be combined with other aesthetic compositions, such as dermal fillers. For example, the composition may be combined with fillers that comprise crosslinked hyaluronic acid (e.g. Juvéderm®). For example, the composition may be combined with fillers that comprise calcium hydroxyapatite (e.g. Radiesse®). For example, the composition may be combined with fillers that comprise poly-L-lactic acid (e.g. Sculptra®). When using PDGF only compositions, the concentration of PDGF applied may be higher than compositions that comprise a carrier. This is because the carrier assists with delivery and reduces the amount of PDGF that is required.

In some embodiments, the composition, whether containing a carrier or not, is prepared using sterile rhPDGF-BB and/or rhPDGF-AB. In some embodiments, the sterile PDGF comprises a pre-formulated sterile PDGF physiologic solution, and in other embodiments the sterile PDGF comprises lyophilized sterile powder containing PDGF reconstituted with sterile water or buffer solution. The buffer solution used to reconstitute the lyophilized rhPDGF-BB and/or rhPDGF-AB or used to prepare the PDGF physiologic solution may comprise, but is not limited to, water, saline, carbonates, phosphates (e.g. phosphate buffered saline), histidine, acetates (e.g. sodium acetate), acidic buffers such as acetic acid and HCL, and organic buffers such as lysine, Tris buffers (e.g. tris(hydroxymethyl)aminoethane), N-2-hydroxyethylpiperazin′-N′-2-ethanesulfonic acid (HEPES), and 3-(N-morpholino) propanesulfonic acid (MOPS). Preferably, the buffer solution is sterile. Buffers can be selected based on biocompatibility with PDGF and the buffer's ability to impede undesirable protein modification. Buffers can additionally be selected based on compatibility with the intended application. In some embodiments, a sodium acetate buffer is used. In some embodiments, a sodium chloride buffer is used. In some embodiments, a phosphate buffered saline is used. In some embodiments, bacteriostatic saline is used. In some embodiments, a phosphate buffered saline and water is used. In some embodiments, a sodium chloride buffer or a sodium acetate buffer is used. In some embodiments, both sodium acetate and sodium chloride are used as the buffer. In some embodiments, the buffer comprises sodium acetate and water. In some embodiments, the buffer comprises sodium chloride and water. The buffers can be employed at different molarities, for example, about 0.1 mM to about 500 mM, about 0.1 mM to about 100 mM, about 1 mM to about 50 mM, about 5 mM to about 40 mM, about 10 mM to about 30 mM, or about 15 mM to about 25 mM, or about 100 mM to about 200 mM or any molarity within these ranges. In some embodiments, an acetate buffer is employed at a molarity of about 20 mM. In some embodiments, the buffer comprises about 20 mM sodium acetate. In some embodiments, the buffer comprises about 20 mM sodium acetate and water. In some embodiments, the buffer comprises about 0.90% w/v (0.15 M) sodium chloride or phosphate-buffered saline. In some embodiments, the buffer comprises about 0.15 M sodium chloride and water. In some embodiments, the buffer comprises about 20 mM sodium acetate, about 0.15 M sodium chloride and water. The composition of the invention thus comprises a PDGF physiologic solution comprising the PDGF in a buffer as described herein. In some embodiments, the composition comprises (in addition to rhPDGF-BB and/or rhPDGF-AB and optionally a carrier) about 20 mM sodium acetate, and optionally water. In some embodiments, the composition comprises (in addition to rhPDGF-BB and/or rhPDGF-AB and optionally a carrier) about 0.15 M sodium chloride, and optionally water. In some embodiments, the composition comprises (in addition to rhPDGF-BB and/or rhPDGF-AB and optionally a carrier) about 20 mM sodium acetate, about 0.15 M sodium chloride, and optionally water. Preferably, the buffer is about 20 mM sodium acetate and water. The buffer or physiologic solution may comprise a pH value of 6.0+/−0.5 (i.e., 5.5-6.5).

As described herein, the composition may be prepared using sterile PDGF, a sterile buffer solution and/or a sterile biocompatible carrier. In some embodiments, the final composition is sterile. This is particularly important when applying the composition to surgical incisions or other skin disruptions that penetrate the skin surface (as described herein).

The carrier may comprise hyaluronic acid, collagen of various types, an alginate, fibrin, gelatin, glycosaminoglycans, polysaccharides, a derivative thereof or a combination thereof. In some embodiments, the carrier comprises collagen. In some embodiments, the carrier comprises alginate. In some embodiments, the carrier comprises fibrin. In some embodiments, the carrier comprises gelatin. In some embodiments, the carrier comprises a glycosaminoglycan. For example, the carrier may comprise hyaluronic acid. In some embodiments, the carrier comprises a polysaccharide. In some embodiments, the carrier comprises a combination of a polysaccharide and an alginate. In some embodiments, the carrier comprises a combination of a polysaccharide and collagen. In some embodiments, the carrier comprises hyaluronic acid and does not comprise any other carrier component as described herein. For example, the carrier may comprise or consist of hyaluronic acid, and does not comprise any alginate, fibrin, gelatin, glycosaminoglycans, or other polysaccharide. For example, the carrier may comprise or consist of hyaluronic acid, and does not comprise collagen. For example, the carrier may comprise or consist of hyaluronic acid, and does not comprise fibrin. For example, the carrier may comprise or consist of hyaluronic acid, and does not comprise any other polysaccharide.

The “collagen” in the rhPDGF-BB and/or rhPDGF-AB-based composition not only provides a matrix or scaffold for cell ingrowth, but it also absorbs proteases that are detrimental to restoring skin or connective tissue integrity or healing. The collagen component may be animal-sourced collagen. It may comprise at least 90% Type I collagen, at least 10% type III collagen, hydrolyzed collagen, monomeric collagen, or crosslinked collagen. It may be lyophilized collagen or gel-form collagen.

The composition described herein has been shown to be particularly effective when using hyaluronic acid (HA) as the carrier. Hyaluronic acid is useful as a carrier because it aids in enhancing skin hydration, elasticity, and ECM repair and/or maintenance. The inventor has also observed that HA allows better penetration and absorption of the composition into the subject's skin and hair follicles than other carriers such as collagen. In particular, the flowable HA assists with delivering the composition directly into the skin or into channels/pores created in the skin following aesthetics procedures (such as micro-needling, micro-injections, micro-coring or laser treatment). Any reference to “hyaluronic acid” used herein includes hyaluronic acid or a physiologically acceptable salt thereof, such as sodium salt, potassium salt, zinc salt, and silver salt, or derivatives or mixtures thereof. Hyaluronic acid may be present at about 0.1% to about 2.5% by weight of the overall composition, such as about 0.1% to about 2.5%, about 0.1% to about 1.0%, about 0.2% to about 1.0%, about 0.3% to 1.0%, about 0.4 to about 1.0%, about 0.5% to about 1.5%, about 0.5% to about 1.0%, about 0.5% to about 0.8% or about 0.6% to 0.8%. The hyaluronic acid may be present at about 0.5% to about 1.0% by weight of the overall composition, such as about 0.5%, about 0.6%, about 0.7%, about 0.8%, about 0.9%, or about 1.0%. Preferably, the hyaluronic acid is present at about 0.75% by weight of the overall composition. Hyaluronic acid is available in a range of molecular weight that span between 4000 to 8,000,000 Daltons. In the composition disclosed herein, the hyaluronic acid has a molecular weight between about 100,000 Da and 1000,000 Da. For example, the molecular weight may be between about 125,000 Da and 1000,000 Da. For example, the molecular weight may be between about 150,000 Da and 900,000 Da. For example, the molecular weight may be between about 200,000 Da to about 850,000 Da. The composition may comprise hyaluronic acid that is a blend of at least two molecular weights. For example, the carrier may comprise a blend of: (i) hyaluronic acid with a weight of between about 100,000 Da and 300,000 Da and (ii) hyaluronic acid with a weight of between about 700,000 Da and 950,000 Da. For example, the carrier may comprise a blend of: (i) hyaluronic acid with a weight of between about 125,000 Da and 275,000 Da and (ii) hyaluronic acid with a weight of between about 775,000 Da and 925,000 Da. For example, the carrier may comprise a blend of: (i) hyaluronic acid with a weight of between about 150,000 Da and 250,000 Da and (ii) hyaluronic acid with a weight of between about 800,000 Da and 900,000 Da. For example, the carrier may comprise a blend of: (i) hyaluronic acid with a weight of between about 100,000 Da and 150,000 Da and (ii) hyaluronic acid with a weight of between about 750,000 Da and 900,000 Da. For example, the carrier may comprise a blend of: (i) hyaluronic acid with a weight of between about 175,000 Da and 225,000 Da and (ii) hyaluronic acid with a weight of between about 825,000 Da and 875,000 Da. For example, the carrier may comprise a blend of: (i) hyaluronic acid with a weight of between about 190,000 Da and 210,000 Da and (ii) hyaluronic acid with a weight of between about 840,000 Da and 860,000 Da. For example, the carrier may comprise a blend of: (i) hyaluronic acid with a weight of about 200,000 Da and (ii) hyaluronic acid with a weight of about 850,000 Da. In some embodiments, the two molecular weights are blended at a ratio of between 30:70 to 70:30. In some embodiments, the two molecular weights are blended at a ratio of between 35:65 to 65:35. In some embodiments, the two molecular weights are blended at a ratio of between 40:60 to 60:40. In some embodiments, the two molecular weights are blended at a ratio of 50:50. Preferably, the hyaluronic acid used to prepare the composition is formulated as a serum or gel. In some embodiments, the hyaluronic acid is a serum. In some embodiments, the hyaluronic acid is a gel. In other embodiments, the hyaluronic acid is in a physiologic solution. The resulting composition prepared using a serum, gel, or solution carrier and a PDGF solution thus has a formulation (e.g. liquid or flowable) that is suitable for both topical application and application by injection.

Further, the PDGF (rhPDGF-BB and/or rhPDGF-AB) used in the (therapeutic or cosmetic) composition of the present invention may be derived from any source such as natural source, synthetic source, or a recombinant source and may be intact or clipped. In accordance with an embodiment, the PDGF is produced by recombinant DNA techniques. When PDGF is produced by recombinant DNA techniques, a DNA sequence encoding a single monomer (e.g., PDGF B-chain or PDGF A-chain), is inserted into cultured cells for expression of the B chain monomer or A chain monomer. The monomer is then extracted and isolated from the cell culture and refolded to form the biologically active homodimer (e.g., PDGF-BB) or the heterodimer (e.g., PDGF-AB), which may be further processed for additional purification. Alternatively, a DNA sequence encoding a single monomer (e.g., PDGF B-chain or PDGF A-chain), is inserted into yeast or mammalian cells for expression of the B chain monomer or A chain monomer. The monomer is then extracted and isolated from the cell culture to form the biologically active homodimer (e.g., PDGF-BB) or the heterodimer (e.g., PDGF-AB), which may be further processed for additional purification. In accordance with an embodiment, the cultured cells are eukaryotic cells such as mammalian or yeast cells, or prokaryotic cells such as E. coli cells. The rhPDGF-BB or rhPDGF-AB produced through these recombinant techniques can be purified in accordance with the techniques outlined in PCT No. WO 2005/077973, for instance. In some embodiments, substantially all of the rhPDGF-BB or rhPDGF-AB included in the compositions described herein are intact non-clipped chains. For example, the rhPDGF-BB included in the composition may be at least about 1%, or at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 97% unclipped rhPDGF-BB on a weight basis. In some embodiments, the PDGF-BB used in the (therapeutic or cosmetic) composition of the present invention is clipped. For example, the rhPDGF-AB included in the composition may be at least about 1%, or at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 97% unclipped rhPDGF-AB on a weight basis. In some embodiments, the PDGF-AB used in the (therapeutic or cosmetic) composition of the present invention is clipped. In some embodiments the composition is a therapeutic composition. In some embodiments, the composition is a cosmetic composition.

In some embodiments, the rhPDGF-BB included in the composition of the present invention is a rhPDGF-BB that comprises or consists essentially of an amino acid sequence having at least about 90%, about 91%, about 92%, about 93%, about 94%, about 95%, about 96%, about 97%, about 98%, about 99%, or about 100% identity to SEQ ID NO. 1, which is provided below:

```
SEQ ID NO. 1:
Ser Leu Gly Ser Leu Thr Ile Ala Glu Pro Ala Met Ile Ala Glu Cys Lys Thr Arg Thr
                                5 10 15 20
Glu Val Phe Glu Ile Ser Arg Arg Leu Ile Asp Arg Thr Asn Ala Asn Phe Leu Val Trp
                                25 30 35 40
pro Pro Cys Val Glu Val Gln Arg Cys Ser Gln Cys Cys Asn Arg Asn Phe Val Gln Cys
                                45 50 55 60
Arg Pro Thr Gln Val Gln Leu Arg Pro Val Gle Val Arg Lys Ile Glu Ile Val Arg Lys
                                65 70 75 80
Lys Pro Ile Phe Lys Lys Ala Thr Val Thr Leu Glu Asp His Leu Ala Cys Lys Cys Glu
                                85 90 95 100
Thr Val Ala Ala Ala Arg Pro Val Thr
                                105
```

In some embodiments, the rhPDGF-BB included in the composition of the present invention comprises or consists essentially of at least about 80%, or at least about 85%, or at least about 90%, or at least about 95%, or at least about 97% of unclipped rhPDGF-BB on a weight basis that comprises or consists essentially of an amino acid sequence having at least about 90%, about 91%, about 92%, about 93%, about 94%, about 95%, about 96%, about 97%, about 98%, about 99%, or about 100% identity to SEQ ID NO. 1. The term “identity” refers to the degree to which two amino acid sequences are identical (on a residue by residue basis) over the comparison window. Identity can be determined by comparing a position in each sequence which can be aligned for the purpose of comparison. Standard homology software such as BLAST can be used. When an equivalent position in the compared sequences is occupied by the same amino acid, then the molecules are identical at that position. Percentage identity refers to a function of the number of identical amino acids at positions shared by the compared sequences. In some embodiments, the rhPDGF included in the composition of the present invention is a PDGF that comprises or consists of a PDGF sequence capable of binding to -A or -B proteins or receptors.

In some embodiments, the rhPDGF-AB included in the composition of the present invention is a rhPDGF-AB that comprises a B-chain comprising an amino acid sequence having at least about 90%, about 91%, about 92%, about 93%, about 94%, about 95%, about 96%, about 97%, about 98%, about 99%, or about 100% identity to SEQ ID NO. 1.

In some embodiments, the rhPDGF-AB included in the composition of the present invention is a rhPDGF-AB that comprises a B-chain derived from SEQ ID NO:1.

In some embodiments, the rhPDGF-AB included in the composition of the present invention is a rhPDGF-AB that comprises an A-chain comprising an amino acid sequence having at least about 90%, about 91%, about 92%, about 93%, about 94%, about 95%, about 96%, about 97%, about 98%, about 99%, or about 100% identity to SEQ ID NO. 2, which is provided below:

MNRCWALFLSLCCYLRLVSAEGDPIPEELYEMLSDHSIRSFDDLQRLLHG
DPGEEDGAELDLNMTRSHSGGELESLARGRRSLGSLTIAEPAMIAECKTR
TEVFEISRRLIDRTNANFLVWPPCVEVQRCSGCCNNRNVQCRPTQVQLRP
VQVRKIEIVRKKPIFKKATVTLEDHLACKCETVAAARPVTRSPGGSQEQR
AKTPQTRVTIRTVRVRRPPKGKHRKFKHTHDKTALKETLGA

In some embodiments, the rhPDGF-AB included in the composition of the present invention is a rhPDGF-AB that comprises an A-chain derived from SEQ ID NO:2.

In some embodiments, the rhPDGF-AB included in the composition of the present invention is a rhPDGF-AB that comprises a B-chain comprising an amino acid sequence having at least about 90%, about 91%, about 92%, about 93%, about 94%, about 95%, about 96%, about 97%, about 98%, about 99%, or about 100% identity to SEQ ID NO. 3, which is provided below:

MRTLACLLLLGCGYLAHVLAEEAEIPREVIERLARSQIHSIRDLQRLLEI
DSVGSEDSLDTSLRAHGVHATKHVPEKRPLPIRRKRSIEEAVPAVCKTRT
VIYEIPRSQVDPTSANFLIWPPCVEVKRCTGCCNTSSVKCQPSRVHHRSV
KVAKVEYVRKKPKLKEVQVRLEEHLECACATTSLNPDYREEDTGRPRESG
KKRKRKRLKPT

In some embodiments, the rhPDGF-AB included in the composition of the present invention is a rhPDGF-AB that comprises a B-chain derived from SEQ ID NO:3.

In some embodiments, the rhPDGF-AB included in the composition of the present invention comprises or consists essentially of at least about 80%, or at least about 85%, or at least about 90%, or at least about 95%, or at least about 97% of unclipped rhPDGF-AB on a weight basis that comprises or consists essentially of an amino acid sequence having at least about 90%, about 91%, about 92%, about 93%, about 94%, about 95%, about 96%, about 97%, about 98%, about 99%, or about 100% identity to SEQ ID NO. 1, 2, and/or 3. In some embodiments, the rhPDGF-AB included in the composition of the present invention comprises or consists essentially of at least about 80%, or at least about 85%, or at least about 90%, or at least about 95%, or at least about 97% of unclipped rhPDGF-AB on a weight basis that comprises or consists essentially of an amino acid sequence having at least about 90%, about 91%, about 92%, about 93%, about 94%, about 95%, about 96%, about 97%, about 98%, about 99%, or about 100% identity to SEQ ID NO. 2. In some embodiments, the rhPDGF-AB included in the composition of the present invention comprises or consists essentially of at least about 80%, or at least about 85%, or at least about 90%, or at least about 95%, or at least about 97% of unclipped rhPDGF-AB on a weight basis that comprises or consists essentially of an amino acid sequence having at least about 90%, about 91%, about 92%, about 93%, about 94%, about 95%, about 96%, about 97%, about 98%, about 99%, or about 100% identity to SEQ ID NO. 3. The term “identity” refers to the degree to which two amino acid sequences are identical (on a residue by residue basis) over the comparison window. Identity can be determined by comparing a position in each sequence which can be aligned for the purpose of comparison. Standard homology software such as BLAST can be used. When an equivalent position in the compared sequences is occupied by the same amino acid, then the molecules are identical at that position. Percentage identity refers to a function of the number of identical amino acids at positions shared by the compared sequences. In some embodiments, the rhPDGF included in the composition of the present invention is a PDGF that comprises or consists of a PDGF sequence capable of binding to -A or -B proteins or receptors.

As used herein, the terms “promoting skin regeneration, rejuvenation and/or repair” or “leading to regeneration, rejuvenation and/or repair of a skin disruption” refers to the process in which the condition of the subject's skin or hair follicle is improved and/or a skin disruption is repaired, including regrowth and restoration of hair. In some embodiments, the subject's skin may have an improved aesthetic appearance. The subject's skin may be intact or damaged. In some embodiments, the subject's skin has a skin disruption which affects its appearance. In some embodiments, promoting or leading to regeneration, rejuvenation and/or repair comprises reducing wrinkles, sagging and/or furrows in the subject's skin and/or stimulating hair growth. In some embodiments, promoting or leading to regeneration, rejuvenation and/or repair comprises reducing pore size in the subject's skin. In some embodiments, promoting or leading to regeneration, rejuvenation and/or repair comprises enhancing tissue volume and/or tone. In some embodiments, promoting or leading to regeneration, rejuvenation and/or repair comprises stimulating hair growth (i.e., improving one or more of total hair count, hair density, mean thickness, and cumulative hair thickness). In some embodiments, promoting or leading to regeneration, rejuvenation and/or repair comprises increasing the elasticity of the skin. In some embodiments, promoting or leading to regeneration, rejuvenation and/or repair comprises reducing sunspots or irregular pigmentation in the subject's skin. In some embodiments, promoting or leading to regeneration, rejuvenation and/or repair comprises reducing sunburn. In some embodiments, promoting or leading to regeneration, rejuvenation and/or repair comprises reducing rough texture of the skin. In some embodiments, promoting or leading to regeneration, rejuvenation and/or repair comprises improving the texture of the skin. In some embodiments, promoting or leading to regeneration, rejuvenation and/or repair comprises reducing dryness of the skin. In some embodiments, promoting or leading to regeneration, rejuvenation and/or repair comprises improving the function of the skin. In some embodiments, promoting or leading to regeneration, rejuvenation and/or repair comprises stimulating cell growth. In some embodiments, promoting or leading to regeneration, rejuvenation and/or repair comprises stimulating angiogenesis. In some embodiments, promoting or leading to regeneration, rejuvenation and/or repair comprises reducing, preventing or improving pain or discomfort experienced by the subject e.g. at a site of a skin disruption. The skilled person can readily determine whether treatment promotes or leads to skin regeneration, rejuvenation and repair using standard methods available in the art. For example, by looking for a reduction in wrinkles, furrows and sagging in the subject's skin; reduced redness or other discoloration, smoother skin, increased tissue volume, reduced redness, reduced swelling, reduced pain or discomfort, reduced pigmentation, reduced scarring, or reduced pore size. The skilled person can readily determine whether the cosmetic treatment promotes or leads to hair follicle regeneration and/or an increase in hair growth by using standard methods available in the art. For example, the skilled person can measure hair follicle regeneration by measuring hair follicle phase based on follicle size and presence of key stems cells involved in hair follicle regeneration, as well as standard methods to measure hair growth and hair growth rate. The skilled person can measure an increase in hair growth by measuring changes in total hair count, hair density, mean thickness, and cumulative hair thickness, such as by assessing hair density and thickness (in mm/cm²) and tracking percentage increases over time.

As used herein, the term “disruption” or “skin disruption” refers to the disorders or deficits of skin and/or hair follicles that may arise from genetic disorders (bullous conditions); heredity; aging; intentional or accidental acute injury such as surgical deficits or traumatic deficits; hormone imbalance; disease (thyroid disease); vitamin deficiency; stress; medical intervention, such as chemotherapy; and surgical interventions such as abdominoplasties and other types of surgical tissue flaps. In some embodiments, the skin disruption is an intentional surgical deficit. For example, the skin disruption may be an incision from a surgical procedure (i.e., a sutured incision site, a glued incision site, a stapled incision, or a surgical scar). For example, the skin disruption may be an incision from a cosmetic surgical procedure or a non-cosmetic surgical procedure. For example, the incision may from a surgery to remove a skin cancer or lesion (such as Mohs micrographic surgery). The incision may be a closed incision (such as an incision closed by sutures, glue or staples). In some embodiments, the skin disruption may be caused by an aesthetic procedure, as described herein, such as micro-needling, micro-injections, micro-coring, dermaplaning, dermal abrasion, dermal filling or laser resurfacing. Further, the “disruption” or “skin disruption” may be the result of aging, inflammation, radiation damage, ultraviolet (e.g., sun) damage, or other damage to the skin or hair follicle. In some embodiments, the skin disruption is caused by aging, inflammation, radiation damage, ultraviolet damage or combinations thereof. In some embodiments, the skin disruption is caused by aging. Skin disruptions that are caused by aging may include hair loss, wrinkles, sagging, and furrows. Skin disruptions that are caused by inflammation, radiation damage, and/or ultraviolet damage may include sunburn, sunspots, irregular pigmentation, rough texture and dryness. The “disruption” or “skin disruption” may be hair loss in a subject's scalp or other location on the body. In some embodiments, the skin disruption is caused by hormonal changes and/or medical conditions. In some embodiments, the disruption is caused by a stressful event. In some embodiments, the disruption is caused by medical intervention, such as chemotherapy or radiation to the head or other part of the body. In some embodiments, the disruption is caused by a genetic condition or heredity, such as male-pattern baldness. In some embodiments, the skin disruption may comprise one or more of: wrinkles, sagging, furrows, sunburn sunspots, irregular pigmentation, rough texture and dryness. Aging and ultraviolet damage can also lead to loss of elasticity in skin, as can significant weight loss. In some embodiments, the skin disruption may also be skin laxity. Skin laxity includes sagging, wrinkles and drooping in the skin. Aging and ultraviolet damage can also lead to an increase in the appearance large skin pores. In some embodiments, the skin disruption may be large skin pores. In some embodiments, the skin disruption may be a scar. The scar may be a surgical scar. The skin disruption may be on any part of the subject's body. In some embodiments, the skin disruption is on the subject's head, face, scalp, chest, neck, arms, hands, and/or legs. In some embodiments, the skin disruption is present on the subject's face, scalp, and/or neck. The skin disruption may affect the aesthetic appearance of the skin. The disruptions may be to intact or damaged skin or tissue. In one example, treatable areas may be at least about 1 cm² in area and less than 5 cm²; between 5-15 cm², or over 15 cm², up to and including thermal or chemical burns that cover most of the body surface area.

In the methods and compositions disclosed herein (e.g. to treat a skin disruption on a subject, such as to improve aesthetics of the skin and/or stimulate hair growth, or to improve pain or discomfort caused by a skin disruption on a subject) the skin disruption does not involve tendon, ligaments, or bone. For example, the skin disruption may be located within the epidermis and dermis, or the epidermis, dermis, and hypodermis. In some embodiment, the skin disruption does not extend below the hypodermis. In some embodiments, the skin disruption extends only into the dermal or subdermal layers of the skin. In some embodiment, the skin disruption does not extend below the subcutaneous tissue. As discussed above, the cellular and molecular mechanisms involved in bone regeneration versus wound healing are very complex, radically different and in many cases in direct conflict. The differing processes involve the formation of radically different extracellular matrices, mineralization versus no mineralization, different cell signaling and different cell types. Furthermore, the repair environments are very different, including vascularity for blood supply, cellular protein production involved in the repair process, different innervation, and different biomechanics (i.e., skin elasticity versus bone rigidity). Therefore, the PDGF composition, dosage ranges and treatment protocols known to treat bone repair inside the body would not be used by one skilled in the art to treat disruptions in the skin. Similarly, the known methods involved in repair of deep tissue wounds (including deep partial thickness or full thickness wounds) typically involve a step of debriding the wound to remove necrotic or infected tissue and subsequently apply a wound dressing to reduce infection. Such steps are not required in the methods and uses disclosed herein. Accordingly, the methods and uses disclosed herein (particularly those performed to improve aesthetics of the skin) do not comprise any step of debriding the skin to remove necrotic or infected tissue. In some embodiments, the methods and uses disclosed herein also do not comprise any step of applying a wound dressing to the treated site.

As used herein, the terms “promoting hair growth”, “stimulating hair growth” or “facilitating hair regrowth” refer to an increase, activation, or reactivation in hair growth in a subject, which results in an increase in one or more of total hair count, hair density, mean thickness, and cumulative hair thickness in a subject post-application when compared to baseline measurements taken from the subject at a previous time or average measurements. In some embodiments, “promoting hair growth” refers to an increase in at least two of total hair count, hair density, mean thickness, and cumulative hair thickness in a subject post-application when compared to baseline measurements taken from the subject at a previous time or average measurements. In some embodiments, “promoting hair growth” refers to an increase in at least three of total hair count, hair density, mean thickness, and cumulative hair thickness in a subject post-application when compared to baseline measurements taken from the subject at a previous time or average measurements. In some embodiments, “promoting hair growth” refers to an increase in total hair count, hair density, mean thickness, and cumulative hair thickness in a subject post-application when compared to baseline measurements taken from the subject at a previous time or average measurements. The skilled person can readily determine whether the cosmetic treatment promotes or leads to an increase in hair growth by using standard methods available in the art, such as by monitoring a subject's hair pattern and volume. For example, hair growth can be determined by measuring changes in total hair count, hair density, mean thickness, and cumulative hair thickness, such as by assessing hair density and thickness (in mm/cm²) and tracking percentage increases over time.

As used herein, the term “promoting hair follicle regeneration” refer to an increase, activation, or reactivation in hair follicles in a subject. The skilled person can readily determine whether the cosmetic treatment promotes or leads to promoting hair follicle regeneration using standard methods available in the art. For example, the skilled person can measure hair follicle regeneration by the methods set forth above for measuring hair growth, as total hair count, density and thickness are increased in hair follicle regeneration. Measurements of the growth phase of each hair, such as hair follicle size and the presence of key stem cells and signaling molecules involved in the regeneration process are also measured to determine whether hair follicle regeneration has occurred.

As used herein, the terms “preventing or reducing pain or discomfort” or “improving pain or discomfort” refers to a reduction or elimination of the pain or discomfort experienced by a subject as a result of a skin disruption. For example, at the site of an incision following a cosmetic or surgical intervention as described herein, such as an incision from facelift surgery. Other skin disruptions that are known to cause pain or discomfort include skin disruptions that are caused by aging, inflammation, radiation damage, ultraviolet damage or combinations thereof, such as sunspots, sunburn, and areas of skin that are dry or rough. The application of an aesthetic procedure such as micro-needling, micro-injections, micro-coring, dermal abrasion, dermaplaning, dermal filler or laser resurfacing can also cause pain or discomfort. The compositions described herein can also be applied in combination with these procedures (as described herein) to reduce/prevent or improve pain or discomfort caused by the procedure. The skilled person can determine whether treatment improves pain or discomfort by monitoring a subject's level of pain or discomfort over time following treatment.

As used herein, the term “preventing or reducing redness, swelling and/or inflammation” refers a reduction in the redness, swelling and/or inflammation experienced by a subject as a result of a skin disruption. For example, at the site of an incision following a cosmetic or surgical intervention as described herein, such as an incision from facelift surgery. In a further example, redness, swelling and/or inflammation may be prevented or reduced at a scar such as a surgical scar or acne scar. The application of an aesthetic procedure such as micro-needling, micro-injections, micro-coring, dermal abrasion, dermaplaning, dermal filler or laser resurfacing can also cause redness, swelling and/or inflammation in the subject's skin. The compositions described herein can also be applied in combination with these procedures (either before, during and/or after, as described herein) to prevent or reduce redness, swelling and/or inflammation caused by the procedure. The skilled person can determine whether treatment reduces redness, swelling and/or inflammation by monitoring the appearance of the subject's skin over time following treatment.

As used herein, the terms “cosmetic treatment” and “non-therapeutic treatment” refer to treating a subject until a cosmetically beneficial result is achieved. These terms do not refer to treating a subject for the purpose of treating, preventing or alleviating a disease or condition, or any symptom(s) of a disease or condition. A cosmetic or non-therapeutic method of the invention does not comprise a method for treatment of the human or animal body by therapy. In particular, the subject seeks non-therapeutic improvements such as enhancement of appearance for aesthetic effect. In some embodiments, a cosmetic or non-therapeutic method of the invention does not comprise a method for treatment of the human or animal body by surgery. Non-surgical methods for administering the cosmetic treatments of the invention are described herein. Further, a cosmetic or non-therapeutic method of the invention does not comprise a diagnostic method practiced on the human or animal body.

As used herein, the terms “the subject is healthy”, “healthy skin”, and “the hair follicles and surrounding skin are healthy” refers to a subject, skin, and hair follicles and surrounding skin that do not suffer from a pathological condition, disease, or medical disorder. The subject may exhibit normal physiological variations that do not constitute a medical condition, such as natural skin aging, wrinkles, dullness, or dryness and natural hair thinning and hair loss.

The concentration of PDGF in embodiments of the present invention can be determined by using an enzyme-linked immunoassay as described in U.S. Pat. Nos. 6,221,625, 5,747,273, and 5,290,708, incorporated herein by reference, or any other assay known in the art for determining PDGF concentration. The (therapeutic or cosmetic) composition disclosed herein comprising rhPDGF-BB and a carrier or rhPDGF-BB in a buffer solution may comprise between about 0.001 mg/mL to about 5 mg/mL or between about 0.1 mg/mL to about 1 mg/mL or between about 0.2 mg/mL to about 0.4 mg/mL of rhPDGF-BB. The rhPDGF-BB in the (therapeutic or cosmetic) composition disclosed herein may comprise about 0.001 mg/mL or about 0.01 mg/mL or about 0.3 mg/mL or about 0.5 mg/mL or about 1.0 mg/mL of rhPDGF-BB. The rhPDGF-BB in the composition disclosed herein may comprise at least about 0.001 mg/mL, or at least about 0.01 mg/mL, or at least about 0.3 mg/mL, or at least about 0.5 mg/mL, or at least about 1.0 mg/mL of rhPDGF-BB. In some embodiments, the (therapeutic or cosmetic) composition contains rhPDGF-BB at a concentration of about 0.001 mg/mL, or about 0.01 mg/mL, or about 0.02 mg/mL, or about 0.03 mg/mL, or about 0.04 mg/mL, or about 0.05 mg/mL, or about 0.06 mg/mL, or about 0.07 mg/mL, or about 0.08 mg/mL, or about 0.09 mg/mL, or about 0.1 mg/mL, or about 0.2 mg/mL, or about 0.25 mg/mL, or about 0.3 mg/mL, or about 0.35 mg/mL, or about 0.4 mg/mL, or about 0.5 mg/mL, or about 0.6 mg/mL, or about 0.7 mg/mL, or about 0.8 mg/mL, or about 0.9 mg/mL, or about 1 mg/mL, or about 2 mg/mL, or about 3 mg/mL, or about 4 mg/mL, or about 5 mg/mL. In some embodiments, the composition contains rhPDGF-BB at a concentration of at least about 0.001 mg/mL, or at least about 0.01 mg/mL, or at least about 0.02 mg/mL, or at least about 0.03 mg/mL, or at least about 0.04 mg/mL, or at least about 0.05 mg/mL, or at least about 0.06 mg/mL, or at least about 0.07 mg/mL, or at least about 0.08 mg/mL, or at least about 0.09 mg/mL, or at least about 0.1 mg/mL, or at least about 0.2 mg/mL, or at least about 0.25 mg/mL, or at least about 0.3 mg/mL, or at least about 0.35 mg/mL, or at least about 0.4 mg/mL, or at least about 0.5 mg/mL, or at least about 0.6 mg/mL, or at least about 0.7 mg/mL, or at least about 0.8 mg/mL, or at least about 0.9 mg/mL, or at least about 1 mg/mL, or about at least 2 mg/mL, or at least about 3 mg/mL, or at least about 4 mg/mL, or at least about 5 mg/mL. In some embodiments, the composition contains rhPDGF-BB at a concentration between about 0.04 mg/mL and 0.08 mg/mL. For example, the composition may contain rhPDGF-BB at a concentration between about 0.05 mg/mL and 0.08 mg/mL. For example, the composition may contain rhPDGF-BB at a concentration between about 0.06 mg/mL and 0.08 mg/mL. For example, the composition may contain rhPDGF-BB at a concentration between about 0.07 mg/mL and 0.08 mg/mL. For example, the composition may contain rhPDGF-BB at a concentration of about 0.075 mg/mL. For example, rhPDGF-BB may be at a final concentration of between about 0.04 mg rhPDGF-BB/mL and 0.08 mg rhPDGF-BB/mL of the composition. For example, rhPDGF-BB may be at a final concentration of between about 0.05 mg rhPDGF-BB/mL and 0.08 mg rhPDGF-BB/mL of the composition. For example, rhPDGF-BB may be at a final concentration of between about 0.06 mg rhPDGF-BB/mL and 0.08 mg rhPDGF-BB/mL of the composition. For example, rhPDGF-BB may be at a final concentration of between about 0.07 mg rhPDGF-BB/mL and 0.08 mg rhPDGF-BB/mL of the composition. For example, rhPDGF-BB may be at a final concentration of about 0.075 mg rhPDGF-BB/mL of the composition. When the composition does not comprise a carrier molecule (i.e., PDGF alone in a buffer solution), then the concentration of PDGF in the composition may be higher because it is not diluted by the carrier molecule. For example, the PDGF only composition may comprise rhPDGF-BB at a concentration of between about 0.01 and 1.0 mg/mL. The composition may comprise rhPDGF-BB at a concentration of between about 0.1 mg/mL and 0.5 mg/mL. The composition may comprise rhPDGF-BB at a concentration of between about 0.2 mg/mL and 0.4 mg/mL. The composition may comprise rhPDGF-BB at a concentration of about 0.3 mg/mL. For example, rhPDGF-BB may be at a final concentration of between about 0.01 mg rhPDGF-BB/mL and 1.0 mg rhPDGF-BB/mL of the composition. For example, rhPDGF-BB may be at a final concentration of between about 0.1 mg rhPDGF-BB/mL and 0.5 mg rhPDGF-BB/mL of the composition. For example, rhPDGF-BB may be at a final concentration of between about 0.2 mg rhPDGF-BB/mL and 0.4 mg rhPDGF-BB/mL of the composition. For example, rhPDGF-BB may be at a concentration of about 0.3 mg rhPDGF-BB/mL of the composition.

In some embodiments, the total amount of rhPDGF-BB included in the (therapeutic or cosmetic) composition is less than about 1000 μg/mL, or less than about 900 μg/mL, or less than about 800 μg/mL, or less than about 700 μg/mL, or less than about 600 μg/mL, or less than about 500 μg/mL, or less than about 400 μg/mL, or less than about 300 μg/mL, or less than about 200 μg/mL, or less than about 100 μg/mL, or less than 90 μg/mL, or less than about 80 μg/mL, or less than about 70 μg/mL, or less than about 60 μg/mL, or less than about 50 μg/mL, or less than about 25 μg/mL, or less than about 10 μg/mL, or less than about 9 μg/mL, or less than about 8 μg/mL, or less than about 7 μg/mL, or less than about 6 μg/mL, or less than about 5 μg/mL, or less than about 2.5 μg/mL, or less than about 1 μg/mL, or less than about 0.1 μg/mL. In some embodiments, the composition comprises between about 1 μg rhPDGF-BB/mL and 10 μg rhPDGF-BB/mL. In some embodiments, the composition comprises between about 1 μg rhPDGF-BB/mL and 9 μg rhPDGF-BB/mL. In some embodiments, the composition comprises between about 1 μg rhPDGF-BB/mL and 8 μg rhPDGF-BB/mL. In some embodiments, the composition comprises between about 1 μg rhPDGF-BB/mL and 7 μg rhPDGF-BB/mL. In some embodiments, the composition comprises between about 1 μg rhPDGF-BB/mL and 6 μg rhPDGF-BB/mL. In some embodiments, the composition comprises between about 1 μg rhPDGF-BB/mL and 5 μg rhPDGF-BB/mL. In some embodiments, the composition comprises between about 1 μg rhPDGF-BB/mL and 80 μg rhPDGF-BB/mL. In some embodiments, the composition comprises between about 1 μg rhPDGF-BB/mL and 100 μg rhPDGF-BB/mL. In some embodiments, the composition comprises between about 0.1 μg rhPDGF-BB/mL and 1,000 μg rhPDGF-BB/mL. In some embodiments, the composition comprises between about 10 μg rhPDGF-BB/mL and 80 μg rhPDGF-BB/mL. In some embodiments, the composition comprises between about 20 μg rhPDGF-BB/mL and 80 μg rhPDGF-BB/mL. In some embodiments, the composition comprises between about 30 μg rhPDGF-BB/mL and 80 μg rhPDGF-BB/mL. In some embodiments, the composition comprises between about 40 μg rhPDGF-BB/mL and 80 μg rhPDGF-BB/mL. In some embodiments, the composition comprises between about 40 μg rhPDGF-BB/mL and 50 μg rhPDGF-BB/mL. In some embodiments, the composition comprises between about 70 μg rhPDGF-BB/mL and 80 μg rhPDGF-BB/mL. In some embodiments, the composition comprises about 75 μg rhPDGF-BB/mL. For example, rhPDGF-BB may be at a final concentration of between about 40 μg rhPDGF-BB/mL and 80 μg rhPDGF-BB/mL of the composition. For example, rhPDGF-BB may be at a final concentration of between about 50 μg rhPDGF-BB/mL and 80 μg rhPDGF-BB/mL of the composition. For example, rhPDGF-BB may be at a final concentration of between about 60 μg rhPDGF-BB/mL and 80 μg rhPDGF-BB/mL of the composition. For example, rhPDGF-BB may be at a final concentration of between about 70 μg rhPDGF-BB/mL and 80 μg rhPDGF-BB/mL of the composition. For example, rhPDGF-BB may be at a final concentration of about 75 μg rhPDGF-BB/mL of the composition. When the final composition does not comprise a carrier molecule (i.e., PDGF alone in a physiologic solution), then the final concentration of PDGF in the composition may be higher because it is not diluted by the carrier molecule. For example, the PDGF only composition may comprise between about 1 μg rhPDGF-BB/mL and 1,000 μg rhPDGF-BB/mL. For example, the PDGF only composition may comprise between about 300 μg rhPDGF-BB/mL and 900 μg rhPDGF-BB/mL. For example, the PDGF only composition may comprise between about 400 μg rhPDGF-BB/mL and 800 μg rhPDGF-BB/mL. For example, the PDGF only composition may comprise between about 500 μg rhPDGF-BB/mL and 700 μg rhPDGF-BB/mL. For example, the PDGF only composition may comprise between about 100 μg rhPDGF-BB/mL and 500 μg rhPDGF-BB/mL. For example, the PDGF only composition may comprise between about 200 μg rhPDGF-BB/mL and 400 μg rhPDGF-BB/mL. For example, the PDGF only composition may comprise about 300 μg rhPDGF-BB/mL. For example, rhPDGF-BB may be at a final concentration of between about 1 μg rhPDGF-BB/mL and 1,000 μg rhPDGF-BB/mL of the composition. For example, rhPDGF-BB may be at a final concentration of between about 300 μg rhPDGF-BB/mL and 900 μg rhPDGF-BB/mL of the composition. For example, rhPDGF-BB may be at a final concentration of between about 400 μg rhPDGF-BB/mL and 800 μg rhPDGF-BB/mL of the composition. For example, rhPDGF-BB may be at a final concentration of between about 500 μg rhPDGF-BB/mL and 700 μg rhPDGF-BB/mL of the composition. For example, rhPDGF-BB may be at a final concentration of between about 100 μg rhPDGF-BB/mL and 500 μg rhPDGF-BB/mL of the composition. For example, rhPDGF-BB may be at a final concentration of between about 200 μg rhPDGF-BB/mL and 800 μg rhPDGF-BB/mL of the composition. For example, rhPDGF-BB may be at a final concentration of between about 200 μg rhPDGF-BB/mL and 400 μg rhPDGF-BB/mL of the composition. For example, rhPDGF-BB may be at a concentration of about 300 μg rhPDGF-BB/mL of the composition.

Various amounts of rhPDGF-BB may be used in the (therapeutic or cosmetic) compositions of the present invention. In some embodiments, the total amount of rhPDGF-BB included in the (therapeutic or cosmetic) composition is less than about 50 mg, or less than about 25 mg, or less than about 10 mg, or less than about 5 mg, or less than about 2.5 mg, or less than about 1 mg, or less than about 0.1 mg, or less than about 0.09 mg, or less than about 0.08 mg, or less than about 0.07 mg, or less than about 0.06 mg, or less than about 0.05 mg, or less than about 0.04 mg, or less than about 0.03 mg, or less than about 0.02 mg, or less than about 0.01 mg, or less than about 0.001 mg. In some embodiments, the total amount of rhPDGF-BB included in the (therapeutic or cosmetic) composition is about 50 mg, or about 25 mg, or about 10 mg, or about 1.0 mg, or about 0.5 mg, or about 0.1 mg, or about 0.09 mg, or about 0.08 mg, or about 0.07 mg, or about 0.06 mg, or about 0.05 mg, or about 0.04 mg, or about 0.03 mg, or about 0.02 mg, or about 0.01 mg, or about 0.001 mg. In some embodiments, the total amount of rhPDGF-BB included in the composition is at least about 50 mg, or at least about 25 mg, or at least about 10 mg, or at least about 1.0 mg, or at least about 0.5 mg, or at least about 0.1 mg, or at least about 0.09 mg, or at least about 0.08 mg, or at least about 0.07 mg, or at least about 0.06 mg, or at least about 0.05 mg, or at least about 0.04 mg, or at least about 0.03 mg, or at least about 0.02 mg, or at least about 0.01 mg, or at least about 0.001 mg.

The (therapeutic or cosmetic) composition disclosed herein comprising rhPDGF-AB and a carrier or rhPDGF-AB in a buffer solution may comprise between about 0.001 mg/mL to about 5 mg/mL or between about 0.1 mg/mL to about 1 mg/mL or between about 0.2 mg/mL to about 0.4 mg/mL of rhPDGF-AB. The rhPDGF-AB in the (therapeutic or cosmetic) composition disclosed herein may comprise about 0.001 mg/mL or about 0.01 mg/mL or about 0.3 mg/mL or about 0.5 mg/mL or about 1.0 mg/mL of rhPDGF-AB. The rhPDGF-AB in the composition disclosed herein may comprise at least about 0.001 mg/mL, or at least about 0.01 mg/mL, or at least about 0.3 mg/mL, or at least about 0.5 mg/mL, or at least about 1.0 mg/mL of rhPDGF-AB. In some embodiments, the (therapeutic or cosmetic) composition contains rhPDGF-AB at a concentration of about 0.001 mg/mL, or about 0.01 mg/mL, or about 0.02 mg/mL, or about 0.03 mg/mL, or about 0.04 mg/mL, or about 0.05 mg/mL, or about 0.06 mg/mL, or about 0.07 mg/mL, or about 0.08 mg/mL, or about 0.09 mg/mL, or about 0.1 mg/mL, or about 0.2 mg/mL, or about 0.25 mg/mL, or about 0.3 mg/mL, or about 0.35 mg/mL, or about 0.4 mg/mL, or about 0.5 mg/mL, or about 0.6 mg/mL, or about 0.7 mg/mL, or about 0.8 mg/mL, or about 0.9 mg/mL, or about 1 mg/mL, or about 2 mg/mL, or about 3 mg/mL, or about 4 mg/mL, or about 5 mg/mL. In some embodiments, the composition contains rhPDGF-AB at a concentration of at least about 0.001 mg/mL, or at least about 0.01 mg/mL, or at least about 0.02 mg/mL, or at least about 0.03 mg/mL, or at least about 0.04 mg/mL, or at least about 0.05 mg/mL, or at least about 0.06 mg/mL, or at least about 0.07 mg/mL, or at least about 0.08 mg/mL, or at least about 0.09 mg/mL, or at least about 0.1 mg/mL, or at least about 0.2 mg/mL, or at least about 0.25 mg/mL, or at least about 0.3 mg/mL, or at least about 0.35 mg/mL, or at least about 0.4 mg/mL, or at least about 0.5 mg/mL, or at least about 0.6 mg/mL, or at least about 0.7 mg/mL, or at least about 0.8 mg/mL, or at least about 0.9 mg/mL, or at least about 1 mg/mL, or about at least 2 mg/mL, or at least about 3 mg/mL, or at least about 4 mg/mL, or at least about 5 mg/mL. In some embodiments, the composition contains rhPDGF-AB at a concentration between about 0.04 mg/mL and 0.08 mg/mL. For example, the composition may contain rhPDGF-AB at a concentration between about 0.05 mg/mL and 0.08 mg/mL. For example, the composition may contain rhPDGF-AB at a concentration between about 0.06 mg/mL and 0.08 mg/mL. For example, the composition may contain rhPDGF-AB at a concentration between about 0.07 mg/mL and 0.08 mg/mL. For example, the composition may contain rhPDGF-AB at a concentration of about 0.075 mg/mL. For example, rhPDGF-AB may be at a final concentration of between about 0.04 mg rhPDGF-AB/mL and 0.08 mg rhPDGF-AB/mL of the composition. For example, rhPDGF-AB may be at a final concentration of between about 0.05 mg rhPDGF-AB/mL and 0.08 mg rhPDGF-AB/mL of the composition. For example, rhPDGF-AB may be at a final concentration of between about 0.06 mg rhPDGF-AB/mL and 0.08 mg rhPDGF-AB/mL of the composition. For example, rhPDGF-AB may be at a final concentration of between about 0.07 mg rhPDGF-AB/mL and 0.08 mg rhPDGF-AB/mL of the composition. For example, rhPDGF-AB may be at a final concentration of about 0.075 mg rhPDGF-AB/mL of the composition. When the composition does not comprise a carrier molecule (i.e., PDGF alone in a buffer solution), then the concentration of PDGF in the composition may be higher because it is not diluted by the carrier molecule. For example, the PDGF only composition may comprise rhPDGF-AB at a concentration of between about 0.01 and 1.0 mg/mL. The composition may comprise rhPDGF-AB at a concentration of between about 0.1 mg/mL and 0.5 mg/mL. The composition may comprise rhPDGF-AB at a concentration of between about 0.2 mg/mL and 0.4 mg/mL. The composition may comprise rhPDGF-AB at a concentration of about 0.3 mg/mL. For example, rhPDGF-AB may be at a final concentration of between about 0.01 mg rhPDGF-AB/mL and 1.0 mg rhPDGF-AB/mL of the composition. For example, rhPDGF-AB may be at a final concentration of between about 0.1 mg rhPDGF-AB/mL and 0.5 mg rhPDGF-AB/mL of the composition. For example, rhPDGF-AB may be at a final concentration of between about 0.2 mg rhPDGF-AB/mL and 0.4 mg rhPDGF-AB/mL of the composition. For example, rhPDGF-AB may be at a concentration of about 0.3 mg rhPDGF-AB/mL of the composition.

In some embodiments, the total amount of rhPDGF-AB included in the (therapeutic or cosmetic) composition is less than about 1000 μg/mL, or less than about 900 μg/mL, or less than about 800 μg/mL, or less than about 700 μg/mL, or less than about 600 μg/mL, or less than about 500 μg/mL, or less than about 400 μg/mL, or less than about 300 μg/mL, or less than about 200 μg/mL, or less than about 100 μg/mL, or less than 90 μg/mL, or less than about 80 μg/mL, or less than about 70 μg/mL, or less than about 60 μg/mL, or less than about 50 μg/mL, or less than about 25 μg/mL, or less than about 10 μg/mL, or less than about 9 μg/mL, or less than about 8 μg/mL, or less than about 7 μg/mL, or less than about 6 μg/mL, or less than about 5 μg/mL, or less than about 2.5 μg/mL, or less than about 1 μg/mL, or less than about 0.1 μg/mL. In some embodiments, the composition comprises between about 1 μg rhPDGF-AB/mL and 10 μg rhPDGF-AB/mL. In some embodiments, the composition comprises between about 1 μg rhPDGF-AB/mL and 9 μg rhPDGF-AB/mL. In some embodiments, the composition comprises between about 1 μg rhPDGF-AB/mL and 8 μg rhPDGF-AB/mL. In some embodiments, the composition comprises between about 1 μg rhPDGF-AB/mL and 7 μg rhPDGF-AB/mL. In some embodiments, the composition comprises between about 1 μg rhPDGF-AB/mL and 6 μg rhPDGF-AB/mL. In some embodiments, the composition comprises between about 1 μg rhPDGF-AB/mL and 5 μg rhPDGF-AB/mL. In some embodiments, the composition comprises between about 1 μg rhPDGF-AB/mL and 80 μg rhPDGF-AB/mL. In some embodiments, the composition comprises between about 1 μg rhPDGF-AB/mL and 100 μg rhPDGF-AB/mL. In some embodiments, the composition comprises between about 0.1 μg rhPDGF-AB/mL and 1,000 μg rhPDGF-AB/mL. In some embodiments, the composition comprises between about 10 μg rhPDGF-AB/mL and 80 μg rhPDGF-AB/mL. In some embodiments, the composition comprises between about 20 μg rhPDGF-AB/mL and 80 μg rhPDGF-AB/mL. In some embodiments, the composition comprises between about 30 μg rhPDGF-AB/mL and 80 μg rhPDGF-AB/mL. In some embodiments, the composition comprises between about 40 μg rhPDGF-AB/mL and 80 μg rhPDGF-AB/mL. In some embodiments, the composition comprises between about 40 μg rhPDGF-AB/mL and 50 μg rhPDGF-AB/mL. In some embodiments, the composition comprises between about 70 μg rhPDGF-AB/mL and 80 μg rhPDGF-AB/mL. In some embodiments, the composition comprises about 75 μg rhPDGF-AB/mL. For example, rhPDGF-AB may be at a final concentration of between about 40 μg rhPDGF-AB/mL and 80 μg rhPDGF-AB/mL of the composition. For example, rhPDGF-AB may be at a final concentration of between about 50 μg rhPDGF-AB/mL and 80 μg rhPDGF-AB/mL of the composition. For example, rhPDGF-AB may be at a final concentration of between about 60 μg rhPDGF-AB/mL and 80 μg rhPDGF-AB/mL of the composition. For example, rhPDGF-AB may be at a final concentration of between about 70 μg rhPDGF-AB/mL and 80 μg rhPDGF-AB/mL of the composition. For example, rhPDGF-AB may be at a final concentration of about 75 μg rhPDGF-AB/mL of the composition. When the final composition does not comprise a carrier molecule (i.e., PDGF alone in a physiologic solution), then the final concentration of PDGF in the composition may be higher because it is not diluted by the carrier molecule. For example, the PDGF only composition may comprise between about 1 μg rhPDGF-AB/mL and 1,000 μg rhPDGF-AB/mL. For example, the PDGF only composition may comprise between about 300 μg rhPDGF-AB/mL and 900 μg rhPDGF-AB/mL. For example, the PDGF only composition may comprise between about 400 μg rhPDGF-AB/mL and 800 μg rhPDGF-AB/mL. For example, the PDGF only composition may comprise between about 500 μg rhPDGF-AB/mL and 700 μg rhPDGF-AB/mL. For example, the PDGF only composition may comprise between about 100 μg rhPDGF-AB/mL and 500 μg rhPDGF-AB/mL. For example, the PDGF only composition may comprise between about 200 μg rhPDGF-AB/mL and 400 μg rhPDGF-AB/mL. For example, the PDGF only composition may comprise about 300 μg rhPDGF-AB/mL. For example, rhPDGF-AB may be at a final concentration of between about 1 μg rhPDGF-AB/mL and 1,000 μg rhPDGF-AB/mL of the composition. For example, rhPDGF-AB may be at a final concentration of between about 300 μg rhPDGF-AB/mL and 900 μg rhPDGF-AB/mL of the composition. For example, rhPDGF-AB may be at a final concentration of between about 400 μg rhPDGF-AB/mL and 800 μg rhPDGF-AB/mL of the composition. For example, rhPDGF-AB may be at a final concentration of between about 500 μg rhPDGF-AB/mL and 700 μg rhPDGF-AB/mL of the composition. For example, rhPDGF-AB may be at a final concentration of between about 100 μg rhPDGF-AB/mL and 500 μg rhPDGF-AB/mL of the composition. For example, rhPDGF-AB may be at a final concentration of between about 200 μg rhPDGF-AB/mL and 800 μg rhPDGF-AB/mL of the composition. For example, rhPDGF-AB may be at a final concentration of between about 200 μg rhPDGF-AB/mL and 400 μg rhPDGF-AB/mL of the composition. For example, rhPDGF-AB may be at a concentration of about 300 μg rhPDGF-AB/mL of the composition.

Various amounts of rhPDGF-AB may be used in the (therapeutic or cosmetic) compositions of the present invention. In some embodiments, the total amount of rhPDGF-AB included in the (therapeutic or cosmetic) composition is less than about 50 mg, or less than about 25 mg, or less than about 10 mg, or less than about 5 mg, or less than about 2.5 mg, or less than about 1 mg, or less than about 0.1 mg, or less than about 0.09 mg, or less than about 0.08 mg, or less than about 0.07 mg, or less than about 0.06 mg, or less than about 0.05 mg, or less than about 0.04 mg, or less than about 0.03 mg, or less than about 0.02 mg, or less than about 0.01 mg, or less than about 0.001 mg. In some embodiments, the total amount of rhPDGF-AB included in the (therapeutic or cosmetic) composition is about 50 mg, or about 25 mg, or about 10 mg, or about 1.0 mg, or about 0.5 mg, or about 0.1 mg, or about 0.09 mg, or about 0.08 mg, or about 0.07 mg, or about 0.06 mg, or about 0.05 mg, or about 0.04 mg, or about 0.03 mg, or about 0.02 mg, or about 0.01 mg, or about 0.001 mg. In some embodiments, the total amount of rhPDGF-AB included in the composition is at least about 50 mg, or at least about 25 mg, or at least about 10 mg, or at least about 1.0 mg, or at least about 0.5 mg, or at least about 0.1 mg, or at least about 0.09 mg, or at least about 0.08 mg, or at least about 0.07 mg, or at least about 0.06 mg, or at least about 0.05 mg, or at least about 0.04 mg, or at least about 0.03 mg, or at least about 0.02 mg, or at least about 0.01 mg, or at least about 0.001 mg.

The concentration of PDGF in the embodiments of the present invention is less than about 10 mg/g, or less than about 5 mg/g or less than about 1 mg/g or less than about 0.5 mg/g, or less than about 0.1 mg/g, or less than about 0.09 mg/g, or less than about 0.08 mg/g, or less than about 0.07 mg/g, or less than about 0.06 mg/g, or less than about 0.05 mg/g, or less than about 0.04 mg/g, or less than about 0.03 mg/g, or less than about 0.02 mg/g, or less than about 0.01 mg/g or less than about 0.001 mg/g. In some embodiments, the concentration of PDGF in the composition of the present invention is between about 0.001 mg/g to about 1 mg/g, or between about 0.05 mg/g to about 5 mg/g, or between about 0.05 mg/g to about 15 mg/g, or between about 0.01 mg/g to about 1 mg/g or between about 0.25 mg/g and about 0.5 mg/g. In some embodiments, the concentration of PDGF in the composition of the present invention is at least about 10 mg/g, or at least about 5 mg/g or at least about 1 mg/g or at least about 0.5 mg/g, or at least about 0.1 mg/g, or at least about 0.09 mg/g, or at least about 0.08 mg/g, or at least about 0.07 mg/g, or at least about 0.06 mg/g, or at least about 0.05 mg/g, or at least about 0.04 mg/g, or at least about 0.03 mg/g, or at least about 0.02 mg/g, or at least about 0.01 mg/g or at least about 0.001 mg/g.

Also provided herein is a therapeutic or cosmetic composition comprising a rhPDGF-BB solution and a carrier, wherein the ratio of the rhPDGF-BB solution to the carrier is between about 0.025 mL solution/cm³ of carrier to about 5 mL solution/cm³ of carrier, or the ratio of rhPDGF-BB to the carrier is between about 1.0 μg rhPDGF-BB/cm³ of carrier to about 750 μg rhPDGF-BB/cm³ of carrier.

Also provided herein is a therapeutic or cosmetic composition comprising a rhPDGF-AB solution and a carrier, wherein the ratio of the rhPDGF-AB solution to the carrier is between about 0.025 mL solution/cm³ of carrier to about 5 mL solution/cm³ of carrier, or the ratio of rhPDGF-AB to the carrier is between about 1.0 μg rhPDGF-AB/cm³ of carrier to about 750 μg rhPDGF-AB/cm³ of carrier.

In some embodiments, when the composition comprises a rhPDGF-BB solution in a carrier, such as hyaluronic acid, the ratio of rhPDGF-BB solution to the carrier is 1:2. In some embodiments, when the composition comprises a rhPDGF-BB solution in a carrier, such as hyaluronic acid, the ratio of rhPDGF-BB solution to the carrier is 1:1. In some embodiments, the ratio of rhPDGF-BB solution to the carrier is 1:3. In some embodiments, the ratio of rhPDGF-BB solution to the carrier is 1:4. In some embodiments, the ratio of rhPDGF-BB solution to the carrier is 1:5. In some embodiments, the ratio of rhPDGF-BB solution to the carrier is 1:6. In some embodiments, the ratio of rhPDGF-BB solution to the carrier is 1:7. In some embodiments, the ratio of rhPDGF-BB solution to the carrier is 1:8. In some embodiments, when the composition comprises a rhPDGF-BB solution in a carrier, such as hyaluronic acid, the ratio of rhPDGF-BB solution to the carrier is 1:9. In some embodiments, the ratio of rhPDGF-BB solution to the carrier is 1:10. In some embodiments, when the composition comprises a rhPDGF-BB solution in a carrier, such as hyaluronic acid, the ratio of rhPDGF-BB solution to the carrier is 2:1. In some embodiments, the ratio of rhPDGF-BB solution to the carrier is 3:1. In some embodiments, the ratio of rhPDGF-BB solution to the carrier is 4:1. In some embodiments, the ratio of rhPDGF-BB solution to the carrier is 5:1. In some embodiments, the ratio of rhPDGF-BB solution to the carrier is 6:1. In some embodiments, the ratio of rhPDGF-BB solution to the carrier is 7:1. In some embodiments, the ratio of rhPDGF-BB solution to the carrier is 8:1.

In some embodiments, when the composition comprises a rhPDGF-AB solution in a carrier, such as hyaluronic acid, the ratio of rhPDGF-AB solution to the carrier is 1:2. In some embodiments, when the composition comprises a rhPDGF-AB solution in a carrier, such as hyaluronic acid, the ratio of rhPDGF-AB solution to the carrier is 1:1. In some embodiments, the ratio of rhPDGF-AB solution to the carrier is 1:3. In some embodiments, the ratio of rhPDGF-AB solution to the carrier is 1:4. In some embodiments, the ratio of rhPDGF-AB solution to the carrier is 1:5. In some embodiments, the ratio of rhPDGF-AB solution to the carrier is 1:6. In some embodiments, the ratio of rhPDGF-AB solution to the carrier is 1:7. In some embodiments, the ratio of rhPDGF-AB solution to the carrier is 1:8. In some embodiments, when the composition comprises a rhPDGF-AB solution in a carrier, such as hyaluronic acid, the ratio of rhPDGF-AB solution to the carrier is 1:9. In some embodiments, the ratio of rhPDGF-AB solution to the carrier is 1:10. In some embodiments, when the composition comprises a rhPDGF-AB solution in a carrier, such as hyaluronic acid, the ratio of rhPDGF-AB solution to the carrier is 2:1. In some embodiments, the ratio of rhPDGF-AB solution to the carrier is 3:1. In some embodiments, the ratio of rhPDGF-AB solution to the carrier is 4:1. In some embodiments, the ratio of rhPDGF-AB solution to the carrier is 5:1. In some embodiments, the ratio of rhPDGF-AB solution to the carrier is 6:1. In some embodiments, the ratio of rhPDGF-AB solution to the carrier is 7:1. In some embodiments, the ratio of rhPDGF-AB solution to the carrier is 8:1.

The present inventor has observed that the composition described herein only requires a single PDGF growth factor (rhPDGF-BB and/or rhPDGF-AB) to be effective for the methods and uses described herein (e.g. in promoting skin repair, rejuvenation and regeneration (such as reducing wrinkles, furrow, sagging, pigmentation, sunspots, rough texture or dryness), promoting hair follicle regeneration and/or stimulating hair growth, reducing, preventing or improving pain or discomfort caused by a skin disruption, and preventing or reducing redness, swelling or inflammation caused by a skin disruption). Other growth factors, such as TGF and EGF are not required in the composition to achieve these effects. In some embodiments, the composition does not comprise growth factors other than PDGF. Consequently, the composition of the invention requiring only a single growth factor is simple to prepare and avoids unnecessary use of multiple growth factors. As described herein, PDGF is able to achieve effective skin repair and regeneration because it promotes: (i) chemotaxis which causes an immediate influx of regenerative cells including stem cells, fibroblasts and smooth muscle that accelerate repair or rejuvenation of skin or connective tissues and improves the quality or integrity of tissues such as skin; (ii) mitogenesis which promotes the proliferation or growth of regenerative cells including stem cells; (iii) angiogenesis which promotes the growth of new capillaries and blood vessels which enhance the tissue environment for healthy tissue growth or rejuvenation or regeneration; (iv) production of collagen, elastin, glycosaminoglycans and hyaluronic acid, all of which are important for maintaining healthy tissue; and (v) an anti-apoptotic effect to improve cell survival. Other growth factors have a distinct mechanism of action and do not achieve the same broad range of regenerative and restoration effects as PDGF. For example, transforming growth factor (e.g. TGF) is known to stimulate inflammatory cells and leads to scar tissue formation with dense collagen. Fibroblast growth factor (e.g. FGF) is known to induce abnormal blood vessel formation. These growth factors are therefore considered to be unsuitable for skin repair and regeneration in aesthetics. VEGF stimulates angiogenesis but does not promote the other regenerative processes that are achieved when using PDGF. Likewise, connective tissue growth factor (CTGF) promotes connective tissue growth (as does PDGF) but does not promote the other regenerative processes that are achieved when using PDGF. VEGF and CTGF are therefore less useful for promoting skin repair, regeneration and restoration than PDGF. Accordingly, in some embodiments, the composition does not comprise any additional growth factor (i.e., does not comprise any growth factors in addition to recombinant human PDGF). In some embodiments, the composition does not comprise any additional growth factor, including any transforming growth factor (e.g. TGF-beta), fibroblast growth factor (e.g. FGF), hepatocyte growth factor (e.g. HGF), vascular endothelial growth factor (e.g. VEGF), connective tissue growth factor (e.g. CTGF), insulin-like growth factor (e.g. IGF), keratinocyte growth factor (e.g. KGF), or epithelial growth factor (e.g. EGF). IGF and EGF have been reported to have some useful benefits for wound healing. In some embodiments, the composition does not comprise VEGF, CTGF, and/or TGF (e.g. TGF-beta). In particular, the composition does not comprise TGF-beta. In particular, the composition does not comprise VEGF. In particular, the composition does not comprise CTGF. In particular, in some embodiments, the composition does not comprise KGF.

The inventor has further observed that certain additional components increase the risk of potential harmful interactions, as well as the expense of preparing the composition. In addition, certain additional components can also reduce the bioavailability of PDGF. For example, compositions that comprise alcohols are considered to have a drying effect on the skin. Other components such as carboxymethylcellulose, guar gum or cellulose can stimulate inflammation. In some embodiments, the composition does not comprise one or more of: alcohol, carboxymethylcellulose, guar gum and cellulose. In some embodiments, the composition does not comprise any alcohol. In some embodiments, the composition does not comprise carboxymethylcellulose. In some embodiments, the composition does not comprise guar gum. In some embodiments, the composition does not comprise cellulose.

Other additional components may however be useful to include in the composition and further assist with promoting skin and hair follicle repair, regeneration and restoration. In some embodiments, the composition described herein further comprises additional components, such as a local anesthetic, including but not limited to, carbocaine, lidocaine, epinephrine or combinations thereof, other PDGFs, and PRP and/or PRF.

The present invention also provides a kit comprising the composition described herein. In some embodiments, the composition is contained within a preformulated syringe. In some embodiments, the invention provides two compositions for use in the methods and uses described herein. One composition comprises the carrier, such as hyaluronic acid. The other composition comprises rhPDGF-BB and/or rhPDGF-AB. These two compositions may be comprised within a kit. In a further aspect, the invention also provides a kit comprising both compositions. The compositions may be each contained within a preformulated syringe.

The composition may be prepared in different formulations according to the intended use. For example, the composition described herein may be formulated as a cream, gel, serum, balm, sun cream, after sun cream, foundation, tinted cream, tinted sun cream, solution, suspension, emulsion, ointment, foam, paste, lotion, powder, soap, surfactant-containing cleansing oil, or spray. Preferably, the composition is formulated as a serum or gel.

### Methods and Uses of the Compositions

The present inventor has observed that the (therapeutic or cosmetic) composition of rhPDGF-BB and/or rhPDGF-AB with or without hyaluronic acid or other carrier disclosed herein stimulates cell growth, angiogenesis or collagen deposition in the skin and enhances tissue volume, which improves the tone and tightening of the skin. This improvement may be beyond an improvement observed in the skin by using hyaluronic acid alone. Further, the composition also stimulates angiogenesis, which further improves the health, texture, fullness or color of the skin. Additionally, the composition reduces discomfort or pain or also facilitates healing following a micro-needling, micro-injections, or micro-coring procedure beyond the comfort or healing that hyaluronic acid alone would result in. The composition also reduces any pain, swelling, redness, inflammation, or scar formation experienced by a patient receiving the treatment and provides a cooling, tingling and soothing effect on the skin and can be applied to the skin before, during and/or after aesthetic procedures, such as micro-needling, micro-injections, and micro-coring to facilitate repair, pain reduction, and healing. Further, the composition promotes hair follicle regeneration and/or hair growth. Thus, the disclosed composition and method improve the function, texture, comfort and/or appearance or aesthetic outcomes in a human being. Moreover, the lower viscosity of the composition comprising rhPDGF-BB and/or rhPDGF-AB, as described herein, compared to other aesthetic treatment protocols (such as PRP and PRF), means that the composition is easier to inject, handle, and use, resulting in reduced discomfort and pain for the patient. It is also easier for the user to apply, thereby improving the overall usability of the treatment. These improved outcomes increase patient satisfaction.

Consequently, the treatment procedure described herein results in more satisfied patients immediately following the treatment of the damaged, diseased or deficient skin or connective tissue, with the medical benefits continuing from several weeks to several months. The compositions and methods disclosed herein heal cosmetic disruptions to the dermal or subdermal layers of the skin and skin appendages, such as hair follicles, in a patient-friendly and consistent manner. As discussed herein, these outcomes are unexpected based on the uses of PDGF known in the art.

In a further aspect, the invention provides methods for treating a subject using the compositions described. In some embodiments, the method may comprise cosmetically treating a subject using the compositions described herein. A cosmetic or non-therapeutic method of the invention does not comprise a method for treatment of the human or animal body by therapy. In particular, the subject seeks non-therapeutic improvements such as enhancement of appearance for aesthetic effect. In some embodiments, a cosmetic or non-therapeutic method of the invention does not comprise a method for treatment of the human or animal body by surgery. Non-surgical methods for administering the cosmetic treatments of the invention are described herein. Further, a cosmetic or non-therapeutic method of the invention does not comprise a diagnostic method practiced on the human or animal body. In a further aspect, the invention provides compositions as described herein for use in a method of treating a subject. In some embodiments, the composition described herein is for use in a method of treating a subject. In a further aspect, the invention provides a use of the compositions described herein in the manufacture of a medicament for treating a subject. All the methods and uses described herein may be performed using any of the compositions described herein.

The term “subject” as used herein refers to any organism commonly vertebrates such as humans and animals which is in need of treatment. The term includes mammals such as primates, livestock animals (e.g. cattle, horses, pigs, sheep, goats), companion animals (e.g. cats, dogs) and laboratory animals (e.g. mice, rabbits, rats). In some embodiments, the subject is a human. In some embodiments, the subject is healthy. In some embodiments, the subject has a skin disruption as described herein. In some embodiments, the subject has an area of hair loss. In some embodiments, the subject has reduced total hair count, hair density, mean thickness, and/or cumulative hair thickness, in comparison to baseline measurements taken from the subject at a previous time or average measurements.

In a further aspect, the invention provides a method for promoting skin regeneration, rejuvenation or repair in a subject, wherein said method comprises: (1) providing a composition comprising or consisting essentially of sterile recombinant human PDGF-BB (rhPDGF-BB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof; and (2) applying the composition to an area of the subject's skin, wherein the carrier delivers biologically active rhPDGF onto or into the subject's skin to regenerate, rejuvenate or repair the subject's skin. The method may be a cosmetic treatment method for promoting skin regeneration, rejuvenation or repair in a subject.

In a further aspect, the invention provides a method for promoting skin regeneration, rejuvenation or repair in a subject, wherein said method comprises: (1) providing a composition comprising or consisting essentially of sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof; and (2) applying the composition to an area of the subject's skin, wherein the carrier delivers biologically active rhPDGF onto or into the subject's skin to regenerate, rejuvenate or repair the subject's skin. The method may be a cosmetic treatment method for promoting skin regeneration, rejuvenation or repair in a subject.

In a further aspect, the invention provides a method for promoting skin regeneration, rejuvenation or repair in a subject, wherein said method comprises: (1) providing a composition comprising or consisting essentially of sterile recombinant human PDGF-BB (rhPDGF-BB) and sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof; and (2) applying the composition to an area of the subject's skin, wherein the carrier delivers biologically active rhPDGF onto or into the subject's skin to regenerate, rejuvenate or repair the subject's skin. The method may be a cosmetic treatment method for promoting skin regeneration, rejuvenation or repair in a subject.

In a further aspect, the invention provides a composition comprising or consisting essentially of sterile recombinant human PDGF-BB (rhPDGF-BB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, for use in promoting skin regeneration, rejuvenation or repair in a subject, wherein the composition is applied to an area of the subject's skin, and wherein the carrier delivers biologically active rhPDGF onto or into the subject's skin, including the hair follicle, to regenerate, rejuvenate or repair the subject's skin. In some embodiments, the skin regeneration, rejuvenation or repair in a subject is for cosmetic purposes.

In a further aspect, the invention provides a composition comprising or consisting essentially of sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, for use in promoting skin regeneration, rejuvenation or repair in a subject, wherein the composition is applied to an area of the subject's skin, and wherein the carrier delivers biologically active rhPDGF onto or into the subject's skin, including the hair follicle, to regenerate, rejuvenate or repair the subject's skin. In some embodiments, the skin regeneration, rejuvenation or repair in a subject is for cosmetic purposes.

In a further aspect, the invention provides a composition comprising or consisting essentially of sterile recombinant human PDGF-BB (rhPDGF-BB) and sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, for use in promoting skin regeneration, rejuvenation or repair in a subject, wherein the composition is applied to an area of the subject's skin, and wherein the carrier delivers biologically active rhPDGF onto or into the subject's skin, including the hair follicle, to regenerate, rejuvenate or repair the subject's skin. In some embodiments, the skin regeneration, rejuvenation or repair in a subject is for cosmetic purposes.

In a further aspect, the invention provides a use of a composition comprising or consisting essentially of sterile recombinant human PDGF-BB (rhPDGF-BB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, in the manufacture of a medicament for promoting skin regeneration, rejuvenation or repair in a subject, wherein the carrier delivers biologically active rhPDGF onto or into the subject's skin to regenerate, rejuvenate or repair the subject's skin.

In a further aspect, the invention provides a use of a composition comprising or consisting essentially of sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, in the manufacture of a medicament for promoting skin regeneration, rejuvenation or repair in a subject, wherein the carrier delivers biologically active rhPDGF onto or into the subject's skin to regenerate, rejuvenate or repair the subject's skin.

In a further aspect, the invention provides a use of a composition comprising or consisting essentially of sterile recombinant human PDGF-BB (rhPDGF-BB) and sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, in the manufacture of a medicament for promoting skin regeneration, rejuvenation or repair in a subject, wherein the carrier delivers biologically active rhPDGF onto or into the subject's skin to regenerate, rejuvenate or repair the subject's skin.

In a further aspect, the invention provides a method for promoting skin regeneration, rejuvenation or repair in a subject, wherein said method comprises: (1) providing a composition comprising or consisting essentially of sterile recombinant human PDGF-BB (rhPDGF-BB) in a physiologic solution; and (2) applying the composition to an area of the subject's skin. The method may be a cosmetic treatment method for promoting skin regeneration, rejuvenation or repair in a subject.

In a further aspect, the invention provides a method for promoting skin regeneration, rejuvenation or repair in a subject, wherein said method comprises: (1) providing a composition comprising or consisting essentially of sterile recombinant human PDGF-AB (rhPDGF-AB) in a physiologic solution; and (2) applying the composition to an area of the subject's skin. The method may be a cosmetic treatment method for promoting skin regeneration, rejuvenation or repair in a subject.

In a further aspect, the invention provides a method for promoting skin regeneration, rejuvenation or repair in a subject, wherein said method comprises: (1) providing a composition comprising or consisting essentially of sterile recombinant human PDGF-BB (rhPDGF-BB) and sterile recombinant human PDGF-AB (rhPDGF-AB) in a physiologic solution; and (2) applying the composition to an area of the subject's skin. The method may be a cosmetic treatment method for promoting skin regeneration, rejuvenation or repair in a subject.

The area of the subject's skin to which the composition is applied may have a skin disruption, as defined herein. For example, the skin disruption may result from aging, inflammation, radiation damage, ultraviolet damage or combinations thereof (such as wrinkles, sagging, furrows, skin laxity, sunburn, sunspots, irregular pigmentation, rough texture, dryness, large pores). For example, the skin disruption may be an incision from a surgical procedure, such as a cosmetic surgical procedure e.g. a facelift. For example, the skin disruption may be a scar as described herein (such as a surgical scar or acne scar). For example, the skin disruption may be hyperpigmentation or melasma. For example, the skin disruption may result from a skin penetrating aesthetic procedure such as micro-needling, micro-injections, micro-coring, dermal abrasion or dermal filling. The area of the subject's skin to which the composition is applied may be scheduled for a skin penetrating aesthetic procedure (e.g. micro-needling, micro-injections, micro-coring, dermal abrasion or dermal filling) that will introduce a skin disruption. In this circumstance the composition is applied in preparation for the procedure to assist with skin regeneration, rejuvenation or repair. In some embodiments, the skin disruption does not involve tendons, ligaments or bones. In some embodiments, the skin extends only into the dermal or subdermal layers of the subject's skin. In some embodiments, the term “skin” includes its appendages, such as hair follicles. The methods and uses for promoting skin regeneration, rejuvenation or repair in a subject may be performed using any composition as described herein. For example, the methods and uses may be performed using a composition that comprises rhPDGF-BB and/or rhPDGF-AB and a carrier comprising hyaluronic acid, as described herein. For example, the rhPDGF-BB may be at a concentration of between about 40 μg rhPDGF-BB/mL and 80 μg rhPDGF-BB/mL of the composition, such as about 75 μg rhPDGF-BB/mL of the composition. For example, the rhPDGF-AB may be at a concentration of between about 40 μg rhPDGF-AB/mL and 80 μg rhPDGF-AB/mL of the composition, such as about 75 μg rhPDGF-AB/mL of the composition. For example, the hyaluronic acid may be present at a concentration of about 0.75 wt % of the composition. In some embodiments, the methods and uses are performed using a composition that comprises rhPDGF-BB and/or rhPDGF-AB in a physiologic solution, without a carrier. For these embodiments, the rhPDGF-BB may be at a concentration of between about 100 μg rhPDGF-BB/mL and 500 μg rhPDGF-BB/mL of the composition, such as about 300 μg rhPDGF-BB/mL of the composition. For these embodiments, the rhPDGF-AB may be at a concentration of between about 100 μg rhPDGF-AB/mL and 500 μg rhPDGF-AB/mL of the composition, such as about 300 μg rhPDGF-AB/mL of the composition. Alternatively, for these embodiments, the rhPDGF-BB may be at a concentration of between about 100 μg rhPDGF-BB/mL and 1,000 μg rhPDGF-BB/mL of the composition. Alternatively, for these embodiments, the rhPDGF-AB may be at a concentration of between about 100 μg rhPDGF-AB/mL and 1,000 μg rhPDGF-AB/mL of the composition.

In a further aspect, the invention provides a method of treating a skin disruption in a subject, wherein said method comprises: (1) providing a composition comprising or consisting essentially of sterile recombinant human PDGF-BB (rhPDGF-BB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof; and (2) applying the composition onto or into the disruption, wherein the carrier delivers biologically active rhPDGF onto or into the disruption leading to regeneration, rejuvenation or repair of the disruption. The method may be a cosmetic method for treating a skin disruption to regenerate, rejuvenate or repair the disruption.

In a further aspect, the invention provides a method of treating a skin disruption in a subject, wherein said method comprises: (1) providing a composition comprising or consisting essentially of sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof; and (2) applying the composition onto or into the disruption, wherein the carrier delivers biologically active rhPDGF onto or into the disruption leading to regeneration, rejuvenation or repair of the disruption. The method may be a cosmetic method for treating a skin disruption to regenerate, rejuvenate or repair the disruption.

In a further aspect, the invention provides a method of treating a skin disruption in a subject, wherein said method comprises: (1) providing a composition comprising or consisting essentially of sterile recombinant human PDGF-BB (rhPDGF-BB) and sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof; and (2) applying the composition onto or into the disruption, wherein the carrier delivers biologically active rhPDGF onto or into the disruption leading to regeneration, rejuvenation or repair of the disruption. The method may be a cosmetic method for treating a skin disruption to regenerate, rejuvenate or repair the disruption.

In a further aspect, the invention provides a method of treating a skin disruption in a subject, wherein the disruption does not involve tendons, ligaments, or bones, wherein said method comprises: (1) providing a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof; and (2) applying the composition onto or into the disruption, wherein the carrier delivers biologically active rhPDGF onto or into the disruption leading to regeneration, rejuvenation or repair of the disruption. The method may be a cosmetic method for treating a skin disruption to regenerate, rejuvenate or repair the disruption. In some embodiments, the composition comprises or consists essentially of sterile rhPDGF-BB in a physiologic solution and a sterile biocompatible carrier. In some embodiments, the composition comprises or consists essentially of sterile rhPDGF-AB in a physiologic solution and a sterile biocompatible carrier. In some embodiments, the composition comprises or consists essentially of sterile rhPDGF-BB and rhPDGF-AB in a physiologic solution and a sterile biocompatible carrier.

In a further aspect, the invention provides a composition comprising or consisting essentially of sterile recombinant human PDGF-BB (rhPDGF-BB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, for use in a method of treating a skin disruption in a subject, wherein the composition is applied onto or into the skin disruption, and wherein the carrier delivers biologically active rhPDGF onto or into the skin disruption to regenerate, rejuvenate or repair the disruption. In some embodiments, the skin disruption does not involve tendons, ligaments, or bones.

In a further aspect, the invention provides a composition comprising or consisting essentially of sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, for use in a method of treating a skin disruption in a subject, wherein the composition is applied onto or into the skin disruption, and wherein the carrier delivers biologically active rhPDGF onto or into the skin disruption to regenerate, rejuvenate or repair the disruption. In some embodiments, the skin disruption does not involve tendons, ligaments, or bones.

In a further aspect, the invention provides a composition comprising or consisting essentially of sterile recombinant human PDGF-BB (rhPDGF-BB) and sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, for use in a method of treating a skin disruption in a subject, wherein the composition is applied onto or into the skin disruption, and wherein the carrier delivers biologically active rhPDGF onto or into the skin disruption to regenerate, rejuvenate or repair the disruption. In some embodiments, the skin disruption does not involve tendons, ligaments, or bones.

In a further aspect, the invention provides a use of a composition comprising or consisting essentially of sterile recombinant human PDGF-BB (rhPDGF-BB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, in the manufacture of a medicament for use in a method of treating a skin disruption in a subject, wherein the carrier delivers biologically active rhPDGF onto or into the skin disruption to regenerate, rejuvenate or repair the disruption. In some embodiments, the skin disruption does not involve tendons, ligaments, or bones.

In a further aspect, the invention provides a use of a composition comprising or consisting essentially of sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, in the manufacture of a medicament for use in a method of treating a skin disruption in a subject, wherein the carrier delivers biologically active rhPDGF onto or into the skin disruption to regenerate, rejuvenate or repair the disruption. In some embodiments, the skin disruption does not involve tendons, ligaments, or bones.

In a further aspect, the invention provides a use of a composition comprising or consisting essentially of sterile recombinant human PDGF-BB (rhPDGF-BB) and sterile recombinant human PDGF-AB (rhPDGF-AB) in a physiologic solution and a sterile biocompatible carrier, wherein the biocompatible carrier is hyaluronic acid, collagen, gelatin, alginate, polysaccharide, glycosaminoglycan, fibrin, a derivative thereof or a combination thereof, in the manufacture of a medicament for use in a method of treating a skin disruption in a subject, wherein the carrier delivers biologically active rhPDGF onto or into the skin disruption to regenerate, rejuvenate or repair the disruption. In some embodiments, the skin disruption does not involve tendons, ligaments, or bones.

In a further aspect, the invention provides a method of treating a skin disruption in a subject, wherein said method comprises: (1) providing a composition comprising or consisting essentially of sterile recombinant human PDGF-BB (rhPDGF-BB) in a physiologic solution, and (2) applying the composition onto or into the disruption leading to regeneration, rejuvenation or repair of the disruption. The method may be a cosmetic method for treating a skin disruption to regenerate, rejuvenate or repair the disruption. In some embodiments, the composition does not comprise a carrier, as described herein. The methods and uses described herein which involve carrier free compositions may be applied by injection, as described herein. In some embodiments, the skin disruption does not involve tendons, ligaments, or bones.

In a further aspect, the invention provides a method of treating a skin disruption in a subject, wherein said method comprises: (1) providing a composition comprising or consisting essentially of sterile recombinant human PDGF-AB (rhPDGF-AB) in a physiologic solution, and (2) applying the composition onto or into the disruption leading to regeneration, rejuvenation or repair of the disruption. The method may be a cosmetic method for treating a skin disruption to regenerate, rejuvenate or repair the disruption. In some embodiments, the composition does not comprise a carrier, as described herein. The methods and uses described herein which involve carrier free compositions may be applied by injection, as described herein. In some embodiments, the skin disruption does not involve tendons, ligaments, or bones.

In a further aspect, the invention provides a method of treating a skin disruption in a subject, wherein said method comprises: (1) providing a composition comprising or consisting essentially of sterile recombinant human PDGF-BB (rhPDGF-BB) and sterile recombinant human PDGF-AB (rhPDGF-AB) in a physiologic solution, and (2) applying the composition onto or into the disruption leading to regeneration, rejuvenation or repair of the disruption. The method may be a cosmetic method for treating a skin disruption to regenerate, rejuvenate or repair the disruption. In some embodiments, the composition does not comprise a carrier, as described herein. The methods and uses described herein which involve carrier free compositions may be applied by injection, as described herein. In some embodiments, the skin disruption does not involve tendons, ligaments, or bones.

In a further aspect, the invention provides a composition comprising or consisting essentially of sterile recombinant human PDGF-BB (rhPDGF-BB) in a physiologic solution, for use in a method of treating a skin disruption in a subject, wherein the composition is applied onto or into the skin disruption to regenerate, rejuvenate or repair the disruption. In some embodiments, the skin disruption does not involve tendons, ligaments, or bones.

In a further aspect, the invention provides a composition comprising or consisting essentially of sterile recombinant human PDGF-AB (rhPDGF-AB) in a physiologic solution, for use in a method of treating a skin disruption in a subject, wherein the composition is applied onto or into the skin disruption to regenerate, rejuvenate or repair the disruption. In some embodiments, the skin disruption does not involve tendons, ligaments, or bones.

In a further aspect, the invention provides a composition comprising or consisting essentially of sterile recombinant human PDGF-BB (rhPDGF-BB) and sterile recombinant human PDGF-AB (rhPDGF-AB) in a physiologic solution, for use in a method of treating a skin disruption in a subject, wherein the composition is applied onto or into the skin disruption to regenerate, rejuvenate or repair the disruption. In some embodiments, the skin disruption does not involve tendons, ligaments, or bones.

In a further aspect, the invention provides a use of a composition comprising or consisting essentially of sterile recombinant human PDGF-BB (rhPDGF-BB) in a physiologic solution, in the manufacture of a medicament for use in a method of treating a skin disruption in a subject, wherein the composition is applied onto or into the skin disruption to regenerate, rejuvenate or repair the disruption. In some embodiments, the skin disruption does not involve tendons, ligaments, or bones.

In a further aspect, the invention provides a use of a composition comprising or consisting essentially of sterile recombinant human PDGF-AB (rhPDGF-AB) in a physiologic solution, in the manufacture of a medicament for use in a method of treating a skin disruption in a subject, wherein the composition is applied onto or into the skin disruption to regenerate, rejuvenate or repair the disruption. In some embodiments, the skin disruption does not involve tendons, ligaments, or bones.

In a further aspect, the invention provides a use of a composition comprising or consisting essentially of sterile recombinant human PDGF-BB (rhPDGF-BB) and sterile recombinant human PDGF-AB (rhPDGF-AB) in a physiologic solution, in the manufacture of a medicament for use in a method of treating a skin disruption in a subject, wherein the composition is applied onto or into the skin disruption to regenerate, rejuvenate or repair the disruption. In some embodiments, the skin disruption does not involve tendons, ligaments, or bones.

The methods and uses for treating a skin disruption described herein may be applied to any skin disruption described herein. For example, the skin disruption may result from aging, inflammation, radiation damage, ultraviolet damage or combinations thereof (such as wrinkles, sagging, furrows, skin laxity, sunburn, sunspots, irregular pigmentation, rough texture, dryness, large pores). For example, the skin disruption may be an incision from a surgical procedure, such as a cosmetic surgical procedure e.g. a facelift. For example, the skin disruption may be a scar as described herein (such as a surgical scar or acne scar). For example, the skin disruption may result from a skin penetrating aesthetic procedure such as micro-needling, micro-injections, micro-coring, dermal abrasion, dermaplaning, or laser treatment. The area of the subject's skin to which the composition is applied may be scheduled for a skin penetrating aesthetic procedure (e.g. micro-needling, micro-injections, micro-coring, dermal abrasion, dermaplaning, or laser treatment) that will introduce a skin disruption. In this circumstance the composition is applied in preparation for the procedure to assist with skin regeneration, rejuvenation or repair. In some embodiments, the skin disruption does not involve tendons, ligaments or bones. In some embodiments, the skin disruption extends only into the dermal or subdermal layers of the subject's skin. The methods and uses for treating a skin disruption in a subject may be performed using any composition as described herein. For example, the methods and uses may be performed using a composition that comprises rhPDGF-BB and/or rhPDGF-AB and a carrier comprising hyaluronic acid. For example, the rhPDGF-BB may have a concentration of between about 40 μg rhPDGF-BB/mL and 80 μg rhPDGF-BB/mL of the composition, such as about 75 μg rhPDGF-BB/mL of the composition. For example, the rhPDGF-AB may have a concentration of between about 40 μg rhPDGF-AB/mL and 80 μg rhPDGF-AB/mL of the composition, such as about 75 μg rhPDGF-AB/mL of the composition. For example, the composition may comprise a hyaluronic acid carrier as described herein, which may be present at a concentration of about 0.75 wt % of the composition. In some embodiments, the composition may comprise PDGF in a physiologic solution without a carrier as described herein. For these embodiments, the rhPDGF-BB may have a concentration of between about 100 μg rhPDGF-BB/mL and 500 μg rhPDGF-BB/mL of the composition, such as about 300 μg rhPDGF-BB/mL of the composition. For these embodiments, the rhPDGF-AB may have a concentration of between about 100 μg rhPDGF-AB/mL and 500 μg rhPDGF-AB/mL of the composition, such as about 300 μg rhPDGF-AB/mL of the composition. Alternatively, for these embodiments, the rhPDGF-BB may have a concentration of between about 100 μg rhPDGF-BB/mL and 1,000 μg rhPDGF-BB/mL of the composition. Alternatively, for these embodiments, the rhPDGF-AB may have a concentration of between about 100 μg rhPDGF-AB/mL and 1,000 μg rhPDGF-AB/mL of the composition.

In some embodiments, the invention provides a method (e.g. cosmetic method) of treating a skin disruption caused by an incision from a surgical procedure (e.g. cosmetic surgery such as a facelift) in a subject, wherein said method comprises: (1) providing a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier comprising hyaluronic acid; and (2) applying the composition (e.g. topically) onto or into the incision, wherein the carrier delivers biologically active rhPDGF onto or into the incision to regenerate, rejuvenate or repair the skin disruption. In some embodiments, the composition comprises or consists essentially of sterile rhPDGF-BB in a physiologic solution and a sterile biocompatible carrier comprising hyaluronic acid. In some embodiments, the composition comprises or consists essentially of sterile rhPDGF-AB in a physiologic solution and a sterile biocompatible carrier comprising hyaluronic acid. In some embodiments, the composition comprises or consists essentially of sterile rhPDGF-BB and rhPDGF-AB in a physiologic solution and a sterile biocompatible carrier comprising hyaluronic acid.

In some embodiments, the invention provides a method (e.g. cosmetic method) of treating a skin disruption caused by aging, inflammation, radiation damage, ultraviolet damage or combinations thereof (such as wrinkles, sagging, furrows, skin laxity, sunburn, sunspots, irregular pigmentation, rough texture, dryness, large pores) in a subject, wherein said method comprises: (1) providing a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier comprising hyaluronic acid; and (2) applying the composition (e.g. topically) onto the skin disruption, wherein the carrier delivers biologically active rhPDGF onto or into the disruption to regenerate, rejuvenate or repair the skin disruption. In some embodiments, the method may be performed before, during and/or after an aesthetic procedure such as micro-needling, micro-injections, micro-coring, dermal abrasion, dermaplaning, dermal filling, or laser resurfacing treatment as described herein. In some embodiments, the composition comprises or consists essentially of sterile rhPDGF-BB in a physiologic solution and a sterile biocompatible carrier comprising hyaluronic acid. In some embodiments, the composition comprises or consists essentially of sterile rhPDGF-AB in a physiologic solution and a sterile biocompatible carrier comprising hyaluronic acid. In some embodiments, the composition comprises or consists essentially of sterile rhPDGF-BB and rhPDGF-AB in a physiologic solution and a sterile biocompatible carrier comprising hyaluronic acid.

In some embodiments, the invention provides a method (e.g. cosmetic method) of treating a skin disruption caused by aging, inflammation, radiation damage, ultraviolet damage or combinations thereof (such as wrinkles, sagging, furrows, skin laxity, sunburn, sunspots, irregular pigmentation, rough texture, dryness, large pores) in a subject, wherein said method comprises: (1) providing a composition comprising or consisting essentially of sterile recombinant human PDGF-BB (rhPDGF-BB) and/or sterile recombinant human PDGF-AB (rhPDGF-AB) in a physiologic solution; and (2) applying the composition (e.g. topically or by injection) onto the skin disruption to regenerate, rejuvenate or repair the skin disruption. In some embodiments, the method may be performed before, during and/or after an aesthetic procedure such as micro-needling, micro-injections, micro-coring, dermal abrasion, dermaplaning, dermal filling, or laser resurfacing treatment as described herein. For example, the PDGF only composition may be added to a dermal filler which is applied to the subject's skin by injection. For example, the PDGF only composition may be applied before, during, or after an aesthetic procedure is performed such as micro-needling, micro-injections, or laser treatment (i.e., ablative or non-ablative). In some embodiments, the composition comprises or consists essentially of sterile rhPDGF-BB in a physiologic solution. In some embodiments, the composition comprises or consists essentially of sterile rhPDGF-AB in a physiologic solution. In some embodiments, the composition comprises or consists essentially of sterile rhPDGF-BB and rhPDGF-AB in a physiologic solution.

In some embodiments, the invention provides a method (e.g. cosmetic method) of treating a scar in a subject, wherein said method comprises: (1) providing a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier comprising hyaluronic acid; and (2) applying the composition (e.g. topically or by injection) onto the scar, wherein the carrier delivers biologically active rhPDGF onto or into the scar to regenerate, rejuvenate or repair the subject's skin. In some embodiments, the method reduces redness, swelling and inflammation at the scar site. In some embodiments, the method reduces visibility of the scar. In some embodiments, the method improves the aesthetic appearance of the scar. In some embodiments, the method may be performed before, during and/or after an aesthetic procedure such as micro-needling, micro-injections, micro-coring, dermal abrasion, dermaplaning, dermal filling, or laser treatment, as described herein. The scar may be a surgical scar or acne scar. In some embodiments, the composition comprises or consists essentially of sterile rhPDGF-BB in a physiologic solution and a sterile biocompatible carrier comprising hyaluronic acid. In some embodiments, the composition comprises or consists essentially of sterile rhPDGF-AB in a physiologic solution and a sterile biocompatible carrier comprising hyaluronic acid. In some embodiments, the composition comprises or consists essentially of sterile rhPDGF-BB and rhPDGF-AB in a physiologic solution and a sterile biocompatible carrier comprising hyaluronic acid.

Methods and uses for treating a skin disruption may comprise reducing wrinkles, sagging and/or furrows in the subject's skin. Methods and uses for treating a skin disruption may comprise reducing large pores in the subject's skin. Methods and uses for treating a skin disruption may comprise enhancing tissue volume and/or tone. Methods and uses for treating a skin disruption may comprise increasing the elasticity of the skin. Methods and uses for treating a skin disruption may comprise reducing skin laxity. Methods and uses for treating a skin disruption may comprise reducing sunspots or irregular pigmentation in the subject's skin. Methods and uses for treating a skin disruption may comprise reducing sunburn in the subject's skin. Methods and uses for treating a skin disruption may comprise reducing rough texture of the skin. Methods and uses for treating a skin disruption may comprise improving the texture of the skin. Methods and uses for treating a skin disruption may comprise reducing dryness of the skin. Methods and uses for treating a skin disruption may comprise improving the function of the skin. Methods and uses for treating a skin disruption may comprise stimulating cell growth to promote regeneration, rejuvenation or repair of the subject's skin. Methods and uses for treating a skin disruption may comprise stimulating angiogenesis to promote regeneration, rejuvenation or repair of the subject's skin. Methods and uses for treating a skin disruption may comprise preventing or reducing scarring of the subject's skin. Methods and uses for treating a skin disruption may comprise improving the aesthetic appearance of the skin.

In a further aspect, the invention provides a method of preventing or reducing pain or discomfort caused by a skin disruption in a subject, wherein said method comprises: (1) providing a composition comprising or consisting essentially of sterile recombinant human PDGF-BB (rhPDGF-BB) in a physiologic solution; and (2) applying the composition to an area of the subject's skin that is affected by or will be affected by a skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a method of preventing or reducing pain or discomfort caused by a skin disruption in a subject, wherein said method comprises: (1) providing a composition comprising or consisting essentially of sterile recombinant human PDGF-AB (rhPDGF-AB) in a physiologic solution; and (2) applying the composition to an area of the subject's skin that is affected by or will be affected by a skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a method of preventing or reducing pain or discomfort caused by a skin disruption in a subject, wherein said method comprises: (1) providing a composition comprising or consisting essentially of sterile recombinant human PDGF-BB (rhPDGF-BB) and sterile recombinant human PDGF-AB (rhPDGF-AB) in a physiologic solution; and (2) applying the composition to an area of the subject's skin that is affected by or will be affected by a skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a composition comprising or consisting essentially of a sterile recombinant human PDGF-BB (rhPDGF-BB) in a physiologic solution for use in a method of preventing or reducing pain or discomfort caused by a skin disruption in a subject, wherein the composition is applied to an area of the subject's skin that is affected by or will be affected by a skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a composition comprising or consisting essentially of a sterile recombinant human PDGF-AB (PDGF-AB) in a physiologic solution for use in a method of preventing or reducing pain or discomfort caused by a skin disruption in a subject, wherein the composition is applied to an area of the subject's skin that is affected by or will be affected by a skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a composition comprising or consisting essentially of a sterile recombinant human PDGF-BB (rhPDGF-BB) and sterile recombinant human PDGF-AB (PDGF-AB) in a physiologic solution for use in a method of preventing or reducing pain or discomfort caused by a skin disruption in a subject, wherein the composition is applied to an area of the subject's skin that is affected by or will be affected by a skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a use of a composition comprising or consisting essentially of sterile recombinant human PDGF-BB (rhPDGF-BB) in a physiologic solution in the manufacture of a medicament for use in a method of preventing or reducing pain or discomfort caused by a skin disruption in a subject, wherein the composition is applied to an area of the subject's skin that is affected by or will be affected by a skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a use of a composition comprising or consisting essentially of sterile recombinant human PDGF-AB (PDGF-AB) in a physiologic solution in the manufacture of a medicament for use in a method of preventing or reducing pain or discomfort caused by a skin disruption in a subject, wherein the composition is applied to an area of the subject's skin that is affected by or will be affected by a skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a use of a composition comprising or consisting essentially of sterile recombinant human PDGF-BB (rhPDGF-BB) and sterile recombinant human PDGF-AB (PDGF-AB) in a physiologic solution in the manufacture of a medicament for use in a method of preventing or reducing pain or discomfort caused by a skin disruption in a subject, wherein the composition is applied to an area of the subject's skin that is affected by or will be affected by a skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a method of improving the pain or discomfort caused by a skin disruption in a subject, wherein said method comprises: (1) providing a composition comprising or consisting essentially of sterile recombinant human PDGF-BB (rhPDGF-BB) in a physiologic solution; and (2) applying the composition onto or into the skin disruption leading to reduced pain or discomfort caused by the disruption. In some embodiments, the composition further comprises a carrier as described herein, e.g. hyaluronic acid.

In a further aspect, the invention provides a method of improving the pain or discomfort caused by a skin disruption in a subject, wherein said method comprises: (1) providing a composition comprising or consisting essentially of sterile recombinant human PDGF-AB (PDGF-AB) in a physiologic solution; and (2) applying the composition onto or into the skin disruption leading to reduced pain or discomfort caused by the disruption. In some embodiments, the composition further comprises a carrier as described herein, e.g. hyaluronic acid.

In a further aspect, the invention provides a method of improving the pain or discomfort caused by a skin disruption in a subject, wherein said method comprises: (1) providing a composition comprising or consisting essentially of sterile recombinant human PDGF-BB (rhPDGF-BB) and sterile recombinant human PDGF-AB (PDGF-AB) in a physiologic solution; and (2) applying the composition onto or into the skin disruption leading to reduced pain or discomfort caused by the disruption. In some embodiments, the composition further comprises a carrier as described herein, e.g. hyaluronic acid.

In a further aspect, the invention provides a method of improving the pain or discomfort caused by a skin disruption in a subject, wherein the disruption does not involve tendons, ligaments, or bones, wherein said method comprises: (1) providing a composition comprising or consisting essentially of sterile recombinant human PDGF-BB (rhPDGF-BB) in a physiologic solution; and (2) applying the composition onto or into the skin disruption leading to reduced pain or discomfort caused by the disruption. In some embodiments, the composition further comprises a carrier as described herein, e.g. hyaluronic acid.

In a further aspect, the invention provides a method of improving the pain or discomfort caused by a skin disruption in a subject, wherein the disruption does not involve tendons, ligaments, or bones, wherein said method comprises: (1) providing a composition comprising or consisting essentially of sterile recombinant human PDGF-AB (PDGF-AB) in a physiologic solution; and (2) applying the composition onto or into the skin disruption leading to reduced pain or discomfort caused by the disruption. In some embodiments, the composition further comprises a carrier as described herein, e.g. hyaluronic acid.

In a further aspect, the invention provides a method of improving the pain or discomfort caused by a skin disruption in a subject, wherein the disruption does not involve tendons, ligaments, or bones, wherein said method comprises: (1) providing a composition comprising or consisting essentially of sterile recombinant human PDGF-BB (rhPDGF-BB) and sterile recombinant human PDGF-AB (PDGF-AB) in a physiologic solution; and (2) applying the composition onto or into the skin disruption leading to reduced pain or discomfort caused by the disruption. In some embodiments, the composition further comprises a carrier as described herein, e.g. hyaluronic acid.

In a further aspect, the invention provides a composition comprising or consisting essentially of sterile recombinant human PDGF-BB (rhPDGF-BB) in a physiologic solution for use in a method of improving the pain or discomfort caused by a skin disruption in a subject, wherein the composition is applied onto or into the skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a composition comprising or consisting essentially of sterile recombinant human PDGF-AB (PDGF-AB) in a physiologic solution for use in a method of improving the pain or discomfort caused by a skin disruption in a subject, wherein the composition is applied onto or into the skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a composition comprising or consisting essentially of sterile recombinant human PDGF-BB (rhPDGF-BB) and sterile recombinant human PDGF-AB (PDGF-AB) in a physiologic solution for use in a method of improving the pain or discomfort caused by a skin disruption in a subject, wherein the composition is applied onto or into the skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a use of a composition comprising or consisting essentially of sterile recombinant human PDGF-BB (rhPDGF-BB) in a physiologic solution in the manufacture of a medicament for use in a method of improving the pain or discomfort caused by a skin disruption in a subject, wherein the composition is applied onto or into the skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a use of a composition comprising or consisting essentially of sterile recombinant human PDGF-AB (PDGF-AB) in a physiologic solution in the manufacture of a medicament for use in a method of improving the pain or discomfort caused by a skin disruption in a subject, wherein the composition is applied onto or into the skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

In a further aspect, the invention provides a use of a composition comprising or consisting essentially of sterile recombinant human PDGF-BB (rhPDGF-BB) and sterile recombinant human PDGF-AB (PDGF-AB) in a physiologic solution in the manufacture of a medicament for use in a method of improving the pain or discomfort caused by a skin disruption in a subject, wherein the composition is applied onto or into the skin disruption to prevent or reduce pain or discomfort caused by the disruption. In some embodiments, the skin disruption does not involve tendons, ligaments, or bones. In some embodiments, the composition further comprises a carrier as described herein, e.g. hyaluronic acid.

The methods and uses for preventing, reducing or improving pain or discomfort caused by a skin disruption may be performed using any composition as described herein. For example, the methods and uses may be performed using a composition that comprises rhPDGF-BB and/or rhPDGF-AB in a physiologic solution without a carrier, as described herein. The physiologic solution may comprise the buffer solution as described. For these embodiments, the rhPDGF-BB may have a concentration of between about 100 μg rhPDGF-BB/mL and 1,000 μg rhPDGF-BB/mL of the composition, such as about 500 μg rhPDGF-BB/mL of the composition. For these embodiments, the rhPDGF-AB may have a concentration of between about 100 μg rhPDGF-AB/mL and 1,000 μg rhPDGF-AB/mL of the composition, such as about 500 μg rhPDGF-AB/mL of the composition. Alternatively, the methods and uses may be performed using a composition that comprises rhPDGF-BB and/or rhPDGF-AB and a carrier, preferably comprising hyaluronic acid, as described herein. For these embodiments, the rhPDGF-BB may have a concentration of between about 40 μg rhPDGF-BB/mL and 80 μg rhPDGF-BB/mL of the composition, such as about 75 μg rhPDGF-BB/mL of the composition. For these embodiments, the rhPDGF-AB may have a concentration of between about 40 μg rhPDGF-AB/mL and 80 μg rhPDGF-AB/mL of the composition, such as about 75 μg rhPDGF-AB/mL of the composition. For example, the hyaluronic acid may be present at a concentration of about 0.75 wt % of the composition.

The methods and uses for preventing, reducing or improving pain or discomfort described herein may be applied to any skin disruption described herein. For example, the methods and uses may be performed to reduce or improve pain or discomfort caused by an incision from a surgical procedure. For example, the incision may result from surgery, such as an incision from facelift surgery. In some embodiments, the invention provides a method (e.g. cosmetic method) of reducing (or improving) pain or discomfort caused by a skin incision resulting from surgery (e.g. cosmetic surgery such as a facelift) in a subject, wherein said method comprises: (1) providing a composition comprising or consisting essentially of recombinant human PDGF-BB (rhPDGF-BB) and/or recombinant human PDGF-AB (rhPDGF-AB) and a biocompatible carrier comprising hyaluronic acid; and (2) applying the composition (e.g. topically) onto or into the incision, wherein the carrier delivers biologically active rhPDGF onto or into the incision to reduce pain or discomfort caused by the incision. In some embodiments, the composition comprises or consists essentially of sterile rhPDGF-BB and a biocompatible carrier comprising hyaluronic acid. In some embodiments, the composition comprises or consists essentially of sterile rhPDGF-AB and a biocompatible carrier comprising hyaluronic acid. In some embodiments, the composition comprises or consists essentially of sterile rhPDGF-BB and rhPDGF-AB and a biocompatible carrier comprising hyaluronic acid.

The methods and uses may be performed to prevent, reduce or improve pain or discomfort caused by an aesthetic procedure such as micro-needling, micro-injections, micro-coring, dermal abrasion, dermaplaning, dermal filling, or laser treatment. For example, the methods and uses may be performed to prevent, reduce or improve pain or discomfort caused by a micro-needling procedure. In some embodiments, the invention provides a method (e.g. cosmetic method) of preventing or reducing (or improving) pain or discomfort caused by a micro-needling procedure performed on a subject's skin, wherein said method comprises: (1) providing a composition comprising or consisting essentially of recombinant human PDGF-BB (rhPDGF-BB) and/or recombinant human PDGF-AB (rhPDGF-AB) and a biocompatible carrier comprising hyaluronic acid; and (2) applying the composition (e.g. topically) onto the subject's skin before, during and/or after a micro-needling procedure is performed (e.g. on the same area of the subject's skin), wherein the carrier delivers biologically active rhPDGF onto the subject's skin to prevent or reduce (or improve) pain or discomfort caused by the procedure. The same method can also be performed to prevent or reduce (or improve) pain or discomfort caused by other skin penetrating aesthetic procedures, such as micro-coring, micro-injections, dermal abrasion, dermaplaning, dermal filling, or laser treatment. In some embodiments, the composition comprises or consists essentially of sterile rhPDGF-BB and a biocompatible carrier comprising hyaluronic acid. In some embodiments, the composition comprises or consists essentially of sterile rhPDGF-AB and a biocompatible carrier comprising hyaluronic acid. In some embodiments, the composition comprises or consists essentially of sterile rhPDGF-BB and rhPDGF-AB and a biocompatible carrier comprising hyaluronic acid.

The methods and uses may be performed to reduce or improve pain or discomfort caused by a skin disruption that results from aging, inflammation, radiation damage, ultraviolet damage or combinations thereof. As described herein, skin disruptions that result from aging, inflammation, radiation damage, and/or ultraviolet damage include rough, dry or itchy skin; sunburn (or other thermal skin damage); and some immunological conditions. In some embodiments, the invention provides a method (e.g. cosmetic method) of reducing (or improving) pain or discomfort caused by a skin disruption that results from aging, inflammation, radiation damage, ultraviolet damage or combinations thereof in a subject, wherein said method comprises: (1) providing a composition comprising or consisting essentially of recombinant human PDGF-BB (rhPDGF-BB) and/or recombinant human PDGF-AB (rhPDGF-AB) and a biocompatible carrier comprising hyaluronic acid; and (2) applying the composition (e.g. topically) onto the skin disruption, wherein the carrier delivers biologically active rhPDGF onto the skin disruption to reduce pain or discomfort caused by the disruption. In some embodiments, the invention provides a method (e.g. cosmetic method) of reducing (or improving) pain or discomfort caused by a skin disruption that results from aging, inflammation, radiation damage, ultraviolet damage or combinations thereof (such as rough, dry and/or itchy skin or sunburn) in a subject, wherein said method comprises: (1) providing a composition comprising or consisting essentially of sterile recombinant human PDGF-BB (rhPDGF-BB) and/or recombinant human PDGF-AB (rhPDGF-AB) in a physiologic solution and a sterile biocompatible carrier comprising hyaluronic acid; and (2) applying the composition (e.g. topically) onto the subject's skin before, during and/or after a micro-needling procedure is performed (e.g. on the same area of the subject's skin), wherein the carrier delivers biologically active rhPDGF onto the skin disruption to reduce pain or discomfort caused by the disruption. In some embodiments, the methods and uses may comprise reducing (or improving) pain or discomfort by reducing roughness of the subject's skin. In some embodiments, the methods and uses may comprise reducing (or improving) pain or discomfort by reducing dryness of the subject's skin. In some embodiments, the methods and uses may comprise reducing (or improving) pain or discomfort by reducing itchiness of a subject's skin. In some embodiments, the methods and uses may comprise reducing (or improving) pain or discomfort caused by sunburn or other thermal skin damage. In some embodiments, as described herein, the composition may be formulated as an after-sun and applied topically to the subject's skin. In some embodiments, the composition comprises or consists essentially of rhPDGF-BB and a biocompatible carrier comprising hyaluronic acid. In some embodiments, the composition comprises or consists essentially of rhPDGF-AB and a biocompatible carrier comprising hyaluronic acid. In some embodiments, the composition comprises or consists essentially of rhPDGF-BB and rhPDGF-AB and a biocompatible carrier comprising hyaluronic acid.

In a further aspect, the invention provides a method of preventing or reducing redness, swelling and/or inflammation caused by a skin disruption in a subject, wherein said method comprises: (1) providing a composition comprising or consisting essentially of sterile recombinant human PDGF-BB (rhPDGF-BB) in a physiologic solution; and (2) applying the composition to an area of the subject's skin that is affected or will be affected by a skin disruption to prevent or reduce redness, swelling and/or inflammation caused by the skin disruption. In some embodiments, the skin disruption does not involve tendons, ligaments, or bones. The method may be a cosmetic treatment method for preventing or reducing redness, swelling and/or inflammation caused by a skin disruption. In some embodiments, the composition further comprises a carrier as described herein, e.g. hyaluronic acid.

In a further aspect, the invention provides a method of preventing or reducing redness, swelling and/or inflammation caused by a skin disruption in a subject, wherein said method comprises: (1) providing a composition comprising or consisting essentially of sterile recombinant human PDGF-AB (rhPDGF-AB) in a physiologic solution; and (2) applying the composition to an area of the subject's skin that is affected or will be affected by a skin disruption to prevent or reduce redness, swelling and/or inflammation caused by the skin disruption. In some embodiments, the skin disruption does not involve tendons, ligaments, or bones. The method may be a cosmetic treatment method for preventing or reducing redness, swelling and/or inflammation caused by a skin disruption. In some embodiments, the composition further comprises a carrier as described herein, e.g. hyaluronic acid.

In a further aspect, the invention provides a method of preventing or reducing redness, swelling and/or inflammation caused by a skin disruption in a subject, wherein said method comprises: (1) providing a composition comprising or consisting essentially of sterile recombinant human PDGF-BB (rhPDGF-BB) and sterile recombinant human PDGF-AB (rhPDGF-AB) in a physiologic solution; and (2) applying the composition to an area of the subject's skin that is affected or will be affected by a skin disruption to prevent or reduce redness, swelling and/or inflammation caused by the skin disruption. In some embodiments, the skin disruption does not involve tendons, ligaments, or bones. The method may be a cosmetic treatment method for preventing or reducing redness, swelling and/or inflammation caused by a skin disruption. In some embodiments, the composition further comprises a carrier as described herein, e.g. hyaluronic acid.

In a further aspect, the invention provides a composition comprising or consisting essentially of sterile recombinant human PDGF-BB (rhPDGF-BB) in a physiologic solution for use in a method of preventing or reducing redness, swelling and/or inflammation caused by a skin disruption in a subject, wherein the composition is applied to an area of the subject's skin that is affected by or will be affected by a skin disruption to prevent or reduce redness, swelling and/or inflammation caused by the disruption. In some embodiments, the skin disruption does not involve tendons, ligaments, or bones. In some embodiments, the composition further comprises a carrier as described herein, e.g. hyaluronic acid.

In a further aspect, the invention provides a composition comprising or consisting essentially of sterile human PDGF-AB (rhPDGF-AB) in a physiologic solution for use in a method of preventing or reducing redness, swelling and/or inflammation caused by a skin disruption in a subject, wherein the composition is applied to an area of the subject's skin that is affected by or will be affected by a skin disruption to prevent or reduce redness, swelling and/or inflammation caused by the disruption. In some embodiments, the skin disruption does not involve tendons, ligaments, or bones. In some embodiments, the composition further comprises a carrier as described herein, e.g. hyaluronic acid.

In a further aspect, the invention provides a composition comprising or consisting essentially of sterile recombinant human PDGF-BB (rhPDGF-BB) and recombinant human PDGF-AB (rhPDGF-AB) in a physiologic solution for use in a method of preventing or reducing redness, swelling and/or inflammation caused by a skin disruption in a subject, wherein the composition is applied to an area of the subject's skin that is affected by or will be affected by a skin disruption to prevent or reduce redness, swelling and/or inflammation caused by the disruption. In some embodiments, the skin disruption does not involve tendons, ligaments, or bones. In some embodiments, the composition further comprises a carrier as described herein, e.g. hyaluronic acid.

In a further aspect, the invention provides a use of a composition comprising or consisting essentially of sterile recombinant human PDGF-BB (rhPDGF-BB) in a physiologic solution in the manufacture of a medicament for use in a method of preventing or reducing redness, swelling and/or inflammation caused by a skin disruption in a subject, wherein the composition is applied to an area of the subject's skin that is affected by or will be affected by a skin disruption to prevent or redness, swelling and/or inflammation caused by the disruption. In some embodiments, the skin disruption does not involve tendons, ligaments, or bones. In some embodiments, the composition further comprises a carrier as described herein, e.g. hyaluronic acid.

In a further aspect, the invention provides a use of a composition comprising or consisting essentially of sterile recombinant human PDGF-AB (rhPDGF-AB) in a physiologic solution in the manufacture of a medicament for use in a method of preventing or reducing redness, swelling and/or inflammation caused by a skin disruption in a subject, wherein the composition is applied to an area of the subject's skin that is affected by or will be affected by a skin disruption to prevent or redness, swelling and/or inflammation caused by the disruption. In some embodiments, the skin disruption does not involve tendons, ligaments, or bones. In some embodiments, the composition further comprises a carrier as described herein, e.g. hyaluronic acid.

In a further aspect, the invention provides a use of a composition comprising or consisting essentially of sterile recombinant human PDGF-BB (rhPDGF-BB) and recombinant human PDGF-AB (rhPDGF-AB) in a physiologic solution in the manufacture of a medicament for use in a method of preventing or reducing redness, swelling and/or inflammation caused by a skin disruption in a subject, wherein the composition is applied to an area of the subject's skin that is affected by or will be affected by a skin disruption to prevent or redness, swelling and/or inflammation caused by the disruption. In some embodiments, the skin disruption does not involve tendons, ligaments, or bones. In some embodiments, the composition further comprises a carrier as described herein, e.g. hyaluronic acid.

The methods and uses for preventing or reducing redness, swelling and/or inflammation caused by a skin disruption may be performed using any composition as described herein. For example, the methods and uses may be performed using a composition that comprises rhPDGF-BB and/or rhPDGF-AB in a physiologic solution without a carrier, as described herein. The physiologic solution may comprise the buffer solution as described. For these embodiments, the rhPDGF-BB may have a concentration of between about 100 μg rhPDGF-BB/mL and 1,000 μg rhPDGF-BB/mL of the composition, such as about 500 μg rhPDGF-BB/mL of the composition. For these embodiments, the rhPDGF-AB may have a concentration of between about 100 μg rhPDGF-AB/mL and 1,000 μg rhPDGF-AB/mL of the composition, such as about 500 μg rhPDGF-AB/mL of the composition. Alternatively, the methods and uses may be performed using a composition that comprises rhPDGF-BB and a carrier, preferably comprising hyaluronic acid, as described herein. For example, the rhPDGF-BB may have a concentration of between about 40 μg rhPDGF-BB/mL and 80 μg rhPDGF-BB/mL of the composition, such as about 75 μg rhPDGF-BB/mL of the composition. For example, the rhPDGF-AB may have a concentration of between about 40 μg rhPDGF-AB/mL and 80 μg rhPDGF-AB/mL of the composition, such as about 75 μg rhPDGF-AB/mL of the composition. For example, the hyaluronic acid may be present at a concentration of about 0.75 wt % of the composition.

The methods and uses for preventing or reducing redness, swelling and/or inflammation described herein may be applied to any skin disruption described herein. The methods and uses may be performed to prevent or reduce redness, swelling and/or inflammation caused by an incision from a surgical procedure. For example, the incision may result from cosmetic surgery, such as an incision from facelift surgery. In some embodiments, the invention provides a method (e.g. cosmetic method) of reducing redness, swelling or inflammation caused by a skin incision resulting from surgery (e.g. cosmetic surgery such as a facelift) in a subject, wherein said method comprises: (1) providing a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier comprising hyaluronic acid; and (2) applying the composition (e.g. topically) onto or into the incision, wherein the carrier delivers biologically active rhPDGF onto or into the incision to reduce redness, swelling or inflammation caused by the incision. In some embodiments, the composition comprises or consists essentially of sterile rhPDGF-BB in a physiologic solution and a sterile biocompatible carrier. In some embodiments, the composition comprises or consists essentially of sterile rhPDGF-AB in a physiologic solution and a sterile biocompatible carrier. In some embodiments, the composition comprises or consists essentially of sterile rhPDGF-BB and rhPDGF-AB in a physiologic solution and a sterile biocompatible carrier.

The methods and uses may be performed to prevent or reduce redness, swelling and/or inflammation of a scar. For example, the scar may be a surgical scar or an acne scar, as described herein. In some embodiments, the invention provides a method (e.g. cosmetic method) of reducing redness, swelling or inflammation of a scar on a subject, wherein said method comprises: (1) providing a composition comprising or consisting essentially of sterile recombinant human PDGF-BB (rhPDGF-BB) and/or recombinant human PDGF-AB (rhPDGF-AB) in a physiologic solution and a sterile biocompatible carrier comprising hyaluronic acid; and (2) applying the composition (e.g. topically or by injection) onto or into the scar, wherein the carrier delivers biologically active rhPDGF onto or into the incision to reduce redness, swelling or inflammation at the scar site. In some embodiments, the method reduces visibility of the scar. In some embodiments, the method improves the aesthetic appearance of the scar. In some embodiments, the composition comprises or consists essentially of sterile rhPDGF-BB in a physiologic solution and a sterile biocompatible carrier comprising hyaluronic acid. In some embodiments, the composition comprises or consists essentially of sterile rhPDGF-AB in a physiologic solution and a sterile biocompatible carrier comprising hyaluronic acid. In some embodiments, the composition comprises or consists essentially of sterile rhPDGF-BB and rhPDGF-AB in a physiologic solution and a sterile biocompatible carrier comprising hyaluronic acid.

The methods and uses may be performed to prevent or reduce redness, swelling and/or inflammation caused by a skin penetrating aesthetic procedure such as micro-needling, micro-injections, micro-coring, dermal abrasion, dermaplaning, dermal filling, or laser treatment. For example, the methods and uses may be performed to prevent or reduce redness, swelling and/or inflammation caused by a micro-needling procedure. In some embodiments, the invention provides a method (e.g. cosmetic method) of reducing redness, swelling or inflammation caused by caused by a micro-needling procedure performed on a subject's skin, wherein said method comprises: (1) providing a composition comprising or consisting essentially of sterile recombinant human PDGF-BB (rhPDGF-BB) and/or recombinant human PDGF-AB (rhPDGF-AB) in a physiologic solution and a sterile biocompatible carrier comprising hyaluronic acid; and (2) applying the composition (e.g. topically) onto the subject's skin before, during and/or after a micro-needling procedure is performed (e.g. on the same area of the subject's skin), wherein the carrier delivers biologically active rhPDGF onto the subject's skin to reduce redness, swelling or inflammation caused by the procedure. The same methods can also be performed to prevent or reduce redness, swelling and/or inflammation caused by other skin penetrating aesthetic procedures, such as micro-coring, micro-injections, dermal abrasion, dermaplaning, dermal filling, or laser treatment. In some embodiments, the composition comprises or consists essentially of sterile rhPDGF-BB in a physiologic solution and a biocompatible carrier comprising hyaluronic acid. In some embodiments, the composition comprises or consists essentially of sterile rhPDGF-AB in a physiologic solution and a biocompatible carrier comprising hyaluronic acid. In some embodiments, the composition comprises or consists essentially of sterile rhPDGF-BB and rhPDGF-AB in a physiologic solution and a biocompatible carrier comprising hyaluronic acid.

In a further aspect, the invention provides a method of promoting hair follicle regeneration and/or hair growth in a subject, wherein said method comprises: (1) providing a composition comprising or consisting essentially of recombinant human PDGF-BB (rhPDGF-BB) in a physiologic solution; and (2) applying the composition onto or into hair follicles and surrounding skin and/or an area of hair loss. In some embodiments, the composition comprises or consists essentially of rhPDGF-BB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-BB is sterile. In some embodiments, the biocompatible carrier is sterile. In some embodiments, the composition is applied topically. In some embodiments, the composition is applied by injection. In some embodiments, the composition is applied by sub-dermal injection. In some embodiments, the composition is applied by micro-needling, micro-injections, laser or other penetration of the skin. In some embodiments, the composition is applied by microchannel microinjector.

In a further aspect, the invention provides a method of promoting hair follicle regeneration and/or hair growth in a subject, wherein said method comprises: (1) providing a composition comprising or consisting essentially of recombinant human PDGF-AB (rhPDGF-AB) in a physiologic solution; and (2) applying the composition onto or into hair follicles and surrounding skin and/or an area of hair loss. In some embodiments, the composition comprises or consists essentially of rhPDGF-AB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-AB is sterile. In some embodiments, the biocompatible carrier is sterile. In some embodiments, the composition is applied topically. In some embodiments, the composition is applied by injection. In some embodiments, the composition is applied by sub-dermal injection. In some embodiments, the composition is applied by micro-needling, micro-injections, laser or other penetration of the skin. In some embodiments, the composition is applied by microchannel microinjector.

In a further aspect, the invention provides a method of promoting hair follicle regeneration and/or hair growth in a subject, wherein said method comprises: (1) providing a composition comprising or consisting essentially of recombinant human PDGF-BB (rhPDGF-BB) and recombinant human PDGF-AB (rhPDGF-AB) in a physiologic solution; and (2) applying the composition onto or into hair follicles and surrounding skin and/or an area of hair loss. In some embodiments, the composition comprises or consists essentially of rhPDGF-BB and rhPDGF-AB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-BB and rhPDGF-AB are sterile. In some embodiments, the biocompatible carrier is sterile. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

In a further aspect, the invention provides a cosmetic method of promoting hair follicle regeneration and/or hair growth in a subject, wherein said method comprises: (1) providing a composition comprising or consisting essentially of recombinant human PDGF-BB (rhPDGF-BB) in a physiologic solution; and (2) applying the composition onto or into hair follicles and surrounding skin and/or an area of hair loss. In some embodiments, the composition comprises or consists essentially of rhPDGF-BB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-BB is sterile. In some embodiments, the biocompatible carrier is sterile. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

In a further aspect, the invention provides a cosmetic method of promoting hair follicle regeneration and/or hair growth in a subject, wherein said method comprises: (1) providing a composition comprising or consisting essentially of recombinant human PDGF-AB (rhPDGF-AB) in a physiologic solution; and (2) applying the composition onto or into hair follicles and surrounding skin and/or an area of hair loss. In some embodiments, the composition comprises or consists essentially of rhPDGF-AB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-AB is sterile. In some embodiments, the biocompatible carrier is sterile. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

In a further aspect, the invention provides a cosmetic method of promoting hair follicle regeneration and/or hair growth in a subject, wherein said method comprises: (1) providing a composition comprising or consisting essentially of recombinant human PDGF-BB (rhPDGF-BB) and recombinant human PDGF-AB (rhPDGF-AB) in a physiologic solution; and (2) applying the composition onto or into hair follicles and surrounding skin and/or an area of hair loss. In some embodiments, the composition comprises or consists essentially of rhPDGF-BB and rhPDGF-AB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-BB and rhPDGF-AB are sterile. In some embodiments, the biocompatible carrier is sterile. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

In a further aspect, the invention provides a composition comprising or consisting essentially of recombinant human PDGF-BB (rhPDGF-BB) in a physiologic solution for use in a method of promoting hair follicle regeneration and/or hair growth in a subject, wherein the composition is applied onto or into hair follicles and surrounding skin and/or an area of hair loss. In some embodiments, the composition comprises or consists essentially of rhPDGF-BB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-BB is sterile. In some embodiments, the biocompatible carrier is sterile. In some embodiments, the method is a cosmetic method. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

In a further aspect, the invention provides a composition comprising or consisting essentially of recombinant human PDGF-AB (rhPDGF-AB) in a physiologic solution for use in a method of promoting hair follicle regeneration and/or hair growth in a subject, wherein the composition is applied onto or into hair follicles and surrounding skin and/or an area of hair loss. In some embodiments, the composition comprises or consists essentially of rhPDGF-AB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-AB is sterile. In some embodiments, the biocompatible carrier is sterile. In some embodiments, the method is a cosmetic method. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

In a further aspect, the invention provides a composition comprising or consisting essentially of recombinant human PDGF-BB (rhPDGF-BB) and recombinant human PDGF-AB (rhPDGF-AB) in a physiologic solution for use in a method of promoting hair follicle regeneration and/or hair growth in a subject, wherein the composition is applied onto or into hair follicles and surrounding skin and/or an area of hair loss. In some embodiments, the composition comprises or consists essentially of rhPDGF-BB and rhPDGF-AB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-BB and rhPDGF-AB are sterile. In some embodiments, the biocompatible carrier is sterile. In some embodiments, the method is a cosmetic method. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

In a further aspect, the invention provides a use of a composition comprising or consisting essentially of recombinant human PDGF-BB (rhPDGF-BB) in a physiologic solution in the manufacture of a medicament for use in a cosmetic method of promoting hair follicle regeneration and/or hair growth in a subject, wherein the composition is applied onto or into hair follicles and surrounding skin and/or an area of hair loss. In some embodiments, the composition comprises or consists essentially of rhPDGF-BB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-BB is sterile. In some embodiments, the biocompatible carrier is sterile. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

In a further aspect, the invention provides a use of a composition comprising or consisting essentially of recombinant human PDGF-AB (rhPDGF-AB) in a physiologic solution in the manufacture of a medicament for use in a cosmetic method of promoting hair follicle regeneration and/or hair growth in a subject, wherein the composition is applied onto or into hair follicles and surrounding skin and/or an area of hair loss. In some embodiments, the composition comprises or consists essentially of rhPDGF-AB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-AB is sterile. In some embodiments, the biocompatible carrier is sterile. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

In a further aspect, the invention provides a use of a composition comprising or consisting essentially of recombinant human PDGF-BB (rhPDGF-BB) and recombinant human PDGF-AB (rhPDGF-AB) in a physiologic solution in the manufacture of a medicament for use in a cosmetic method of promoting hair follicle regeneration and/or hair growth in a subject, wherein the composition is applied onto or into hair follicles and surrounding skin and/or an area of hair loss. In some embodiments, the composition comprises or consists essentially of rhPDGF-BB and rhPDGF-AB in a physiologic solution and a biocompatible carrier. In some embodiments, the biocompatible carrier is hyaluronic acid, collagen, gelatin, alginate, polysaccharide, glycosaminoglycan, fibrin, a derivative thereof or a combination thereof. In some embodiments, the biocompatible carrier is hyaluronic acid. In some embodiments, the rhPDGF-BB and rhPDGF-AB are sterile. In some embodiments, the biocompatible carrier is sterile. In some embodiments, the composition is applied topically. In some embodiments, the composition is applied by injection. In some embodiments, the composition is applied by sub-dermal injection. In some embodiments, the composition is applied by micro-needling, laser or other penetration of the skin. In some embodiments, the composition is applied by micro-injections. In some embodiments, the composition is applied by microchannel microinjector.

All the methods and uses described herein may be performed using the composition as described herein. In some embodiments, the composition may comprise rhPDGF-BB and/or rhPDGF-AB (as described herein) in a physiologic solution without a carrier. For these embodiments, the composition preferably comprises between about 100 μg/mL and 500 μg/mL rhPDGF-BB, such as about 300 μg/mL rhPDGF-BB. For these embodiments, the rhPDGF-BB may have a concentration of between about 100 μg rhPDGF-BB/mL and 500 μg rhPDGF-BB/mL of the composition, such as about 300 μg rhPDGF-BB/mL of the composition. Alternatively, for these embodiments, the rhPDGF-BB may have a concentration of between about 100 μg rhPDGF-BB/mL and 1,000 μg rhPDGF-BB/mL of the composition. For these embodiments, the composition preferably comprises between about 100 μg/mL and 500 μg/mL rhPDGF-AB, such as about 300 μg/mL rhPDGF-AB. For these embodiments, the rhPDGF-AB may have a concentration of between about 100 μg rhPDGF-AB/mL and 500 μg rhPDGF-AB/mL of the composition, such as about 300 μg rhPDGF-AB/mL of the composition. Alternatively, for these embodiments, the rhPDGF-AB may have a concentration of between about 100 μg rhPDGF-AB/mL and 1,000 μg rhPDGF-AB/mL of the composition. In some embodiments, the composition may comprise rhPDGF-BB and/or rhPDGF-AB in a physiologic solution (as described herein) and a carrier (as described), preferably hyaluronic acid, as described herein. For example, the composition may comprise between about 40 μg/mL and 80 μg/mL rhPDGF-BB, e.g. about 75 μg/mL rhPDGF-BB. For example, the rhPDGF-BB may have a concentration of between about 40 μg rhPDGF-BB/mL and 80 μg rhPDGF-BB/mL of the composition, e.g. about 75 μg rhPDGF-BB/mL of the composition. For example, the composition may comprise between about 40 μg/mL and 80 μg/mL rhPDGF-AB, e.g. about 75 μg/mL rhPDGF-AB. For example, the rhPDGF-AB may have a concentration of between about 40 μg rhPDGF-AB/mL and 80 μg rhPDGF-AB/mL of the composition, e.g. about 75 μg rhPDGF-AB/mL of the composition. In some embodiments, the composition may comprise: (i) hyaluronic acid at a concentration of between about 0.1 wt % and 1.5 wt %; (ii) rhPDGF-BB at a concentration of between about 40 μg/mL and 80 μg/mL, (iii) between about 10 mM and 30 mM (e.g. about 20 mM) sodium acetate; and (iv) water. For example, the composition may comprise: (i) hyaluronic acid at a concentration of between about 0.5 wt % and 1 wt %; (ii) rhPDGF-BB at a concentration of between about 40 μg/mL and 80 μg/mL; (iii) between about 10 mM and 30 mM (e.g. 20 mM) sodium acetate; and (iv) water. For example, the composition may comprise: (i) hyaluronic acid at a concentration of about 0.75 wt %; (ii) rhPDGF-BB at a concentration of about 75 μg/mL; (iii) about 20 mM sodium acetate; and (iv) water. In some embodiments, the composition may comprise: (i) hyaluronic acid at a concentration of between about 0.1 wt % and 1.5 wt %; (ii) rhPDGF-AB at a concentration of between about 40 μg/mL and 80 μg/mL, (iii) between about 10 mM and 30 mM (e.g. about 20 mM) sodium acetate; and (iv) water. For example, the composition may comprise: (i) hyaluronic acid at a concentration of between about 0.5 wt % and 1 wt %; (ii) rhPDGF-AB at a concentration of between about 40 μg/mL and 80 μg/mL; (iii) between about 10 mM and 30 mM (e.g. 20 mM) sodium acetate; and (iv) water. For example, the composition may comprise: (i) hyaluronic acid at a concentration of about 0.75 wt %; (ii) rhPDGF-AB at a concentration of about 75 μg/mL; (iii) about 20 mM sodium acetate; and (iv) water. Preferably, the composition does not comprise any additional growth factor (i.e., does not comprise any growth factor in addition to recombinant human PDGF (rhPDGF)). In some embodiments, the composition does not comprise any additional growth factors, including any transforming growth factor (e.g. TGF-beta), fibroblast growth factor (e.g. FGF), vascular endothelial growth factor (e.g. VEGF), connective tissue growth factor (e.g. CTGF), hepatocyte growth factor (e.g. HGF), insulin-like growth factor (e.g. IGF), keratinocyte growth factor (e.g. KGF), or epithelial growth factor (e.g. EGF). In particular, the composition does not comprise TGF-beta, VEGF, KGF and/or CTGF.

Water may be present at about 0.1% to about 99% by weight of the overall composition, such as at least about 10 wt %, or at least about 20 wt %, or at least about 30 wt %, or at least about 40 wt %, or at least about 50 wt %, or at least about 60 wt %, or at least about 70 wt %, or at least about 80 wt %, or at least about 85 wt %, or at least about 90 wt %, or at least about 91 wt %, or at least about 92 wt %, or at least about 93 wt %, or at least about 94 wt %, or at least about 95 wt %, or at least about 96 wt %, or at least about 97 wt %, or at least about 98 wt %, or at least about 99 wt %.

All the methods and uses described herein may be applied to a skin disruption as described herein. For example, the skin disruption may result from aging, inflammation, radiation damage, ultraviolet damage or combinations thereof (such as wrinkles, sagging, furrows, skin laxity, sunburn, sunspots, irregular pigmentation, rough texture, dryness, large pores). For example, the skin disruption may be an incision from a surgical procedure, such as a cosmetic surgical procedure e.g. a facelift, abdominoplasty or breast reconstruction, augmentation or reduction surgery. For example, the skin disruption may be a scar, such as a surgical scar or acne scar. For example, the skin disruption may result from a skin penetrating aesthetic procedure such as micro-needling, micro-injections, micro-coring, dermal abrasion, dermaplaning, dermal filling, or laser treatment. The area of the subject's skin to which the composition is applied may be scheduled for a skin penetrating aesthetic procedure (e.g. micro-needling, micro-injections, micro-coring, dermal abrasion, dermaplaning or laser treatment) that will introduce a skin disruption. In this circumstance the composition is applied in preparation for the procedure to assist with skin regeneration, rejuvenation or repair. In some embodiments, the skin disruption does not involve tendons, ligaments or bones. In some embodiments, the skin disruption extends only into the dermal or subdermal layers of the subject's skin. In some embodiments, the skin disruption is located within the epidermis and dermis, or the epidermis, dermis, and hypodermis. In some embodiment, the skin disruption does not extend below the hypodermis. In some embodiment, the skin disruption does not extend below the subcutaneous tissue.

The methods and uses described herein may be cosmetic treatment methods performed to: (i) improve the appearance or aesthetics of the subject's skin; (ii) improve function of the subject's skin; (iii) stimulate cell growth (e.g. to promote skin regeneration, rejuvenation or repair); (iv) reduce the pain and/or discomfort of a skin disruption; (v) stimulate angiogenesis (e.g. to promote skin regeneration, rejuvenation or repair); (vi) improve texture of the subject's skin; (vii) to enhance volume and/or tone of the subject's skin; (viii) prevent or reduce scarring of the subject's skin; (ix) reduce redness, swelling and/or inflammation of a skin disruption; (x) promote hair follicle regeneration and/or (xi) promote hair growth in a subject. In some embodiments, the composition is applied to the skin disruption to improve appearance or aesthetics of the subject's skin. In some embodiments, the composition is applied to the skin disruption to improve function of the subject's skin. In some embodiments, the composition is applied to the skin disruption to stimulate cell growth (e.g. to promote skin regeneration, rejuvenation or repair). In some embodiments, the composition is applied to the skin disruption to reduce the pain and/or discomfort of a skin disruption. In some embodiments, the composition is applied to the skin disruption to stimulate angiogenesis (e.g. to promote skin regeneration, rejuvenation or repair). In some embodiments, the composition is applied to the skin disruption to improve texture of the subject's skin. In some embodiments, the composition is applied to the skin disruption to enhance volume and/or tone of the subject's skin. For example, the composition may reduce wrinkles, furrows and/or sagging. For example, the composition may reduce sunspots or irregular pigmentation in the subject's skin. For example, the composition may reduce sunburn. For example, the composition may increase the elasticity of the subject's skin. For example, the composition may decrease laxity in the subject's skin. For example, the composition may reduce rough texture of the skin. For example, the composition may reduce dryness of the skin. For example, the composition may improve itchy skin. In some embodiments, the composition is applied to the skin disruption to prevent or reduce scarring of the subject's skin. In some embodiments, the composition is applied onto or into hair follicles and surrounding skin and/or an area of hair loss to promote hair follicle regeneration and/or hair growth in a subject. For example, the composition may lead to hair growth. For example, the composition may lead to an increase in one or more of total hair count, hair density, mean thickness, and cumulative hair thickness in a subject post-application when compared to baseline measurements taken from the subject at a previous time or average measurements.

In some embodiments, the methods and uses described herein further comprise a step of preparing the skin or other skin disruption site, hair follicles and surrounding skin, and/or an area of hair loss prior to applying the composition. In some embodiments, the skin or skin disruption, hair follicles and surrounding skin, and/or an area of hair loss is numbed with a numbing agent, such as lidocaine and disinfected with a disinfectant solution, such as alcohol. In some embodiments, the skin or other disruption, hair follicles and surrounding skin, and/or an area of hair loss is cleansed with soap prior to application of the composition.

The methods and uses described herein may further comprise an initial step of forming the composition. In some embodiments, the method or use comprises forming the composition by placing sterile recombinant human PDGF-BB (rhPDGF-BB) and/or sterile recombinant human PDGF-AB (rhPDGF-AB) in a physiologic solution. For example, the methods and uses described herein may comprise an initial step of forming a composition comprising or consisting essentially of sterile recombinant human PDGF-BB (rhPDGF-BB) in a physiologic solution. For example, the methods and uses described herein may comprise an initial step of forming a composition comprising or consisting essentially of sterile recombinant human PDGF-AB (rhPDGF-AB) in a physiologic solution. In some embodiments, the method or use comprises forming the composition by combining: (i) sterile recombinant human PDGF-BB (rhPDGF-BB) in a physiologic solution and (ii) a sterile biocompatible carrier, as described herein. In some embodiments, the method or use comprises forming the composition by combining: (i) sterile recombinant human PDGF-AB (rhPDGF-AB) in a physiologic solution and (ii) a sterile biocompatible carrier, as described herein. For example, the methods and uses described may comprise an initial step of forming a composition comprising or consisting essentially of (i) sterile recombinant human PDGF-BB (rhPDGF-BB) in a physiologic solution and (ii) a sterile biocompatible carrier, wherein the biocompatible carrier is a hyaluronic acid, collagen, gelatin, alginate, polysaccharide, glycosaminoglycan, fibrin, a derivative thereof or a combination thereof. For example, the methods and uses described may comprise an initial step of forming a composition comprising or consisting essentially of (i) sterile recombinant human PDGF-AB (rhPDGF-AB) in a physiologic solution and (ii) a sterile biocompatible carrier, wherein the biocompatible carrier is a hyaluronic acid, collagen, gelatin, alginate, polysaccharide, glycosaminoglycan, fibrin, a derivative thereof or a combination thereof.

In some embodiments, the method of treating a skin disruption on a subject as described herein comprises: (i) forming a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier, wherein the biocompatible carrier is a hyaluronic acid, collagen, gelatin, alginate, polysaccharide, glycosaminoglycan, fibrin, a derivative thereof or a combination thereof; and (ii) applying the composition onto or into the skin disruption, wherein the carrier delivers biologically active rhPDGF onto or into the skin disruption leading to regeneration, rejuvenation or repair of the skin disruption. In some embodiments, the composition comprises or consists essentially of sterile rhPDGF-BB in a physiologic solution and a sterile biocompatible carrier. In some embodiments, the composition comprises or consists essentially of sterile rhPDGF-AB in a physiologic solution and a sterile biocompatible carrier. In some embodiments, the composition comprises or consists essentially of sterile rhPDGF-BB and rhPDGF-AB in a physiologic solution and a sterile biocompatible carrier.

In some embodiments, the method of improving the pain or discomfort caused by a skin disruption or reducing/preventing pain or discomfort caused by a skin disruption as described herein comprises: (i) forming a composition comprising or consisting essentially of sterile recombinant human PDGF-BB (rhPDGF-BB) and/or sterile recombinant human PDGF-AB (rhPDGF-AB) in a physiologic solution; and (ii) applying the composition onto or into the skin disruption leading to reduced pain or discomfort caused by the skin disruption. In some embodiments, the composition comprises or consists essentially of sterile rhPDGF-BB in a physiologic solution. In some embodiments, the composition comprises or consists essentially of sterile rhPDGF-AB in a physiologic solution. In some embodiments, the composition comprises or consists essentially of sterile rhPDGF-BB and rhPDGF-AB in a physiologic solution.

In some embodiments, the method of improving the pain or discomfort caused by a skin disruption or reducing/preventing pain or discomfort caused by a skin disruption as described herein comprises: (i) forming a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier, wherein the biocompatible carrier is a hyaluronic acid, collagen, gelatin, alginate, polysaccharide, glycosaminoglycan, fibrin, a derivative thereof or a combination thereof; and (ii) applying the composition onto or into the skin disruption leading to reduced pain or discomfort caused by the skin disruption. In some embodiments, the composition comprises or consists essentially of sterile rhPDGF-BB in a physiologic solution and a sterile biocompatible carrier. In some embodiments, the composition comprises or consists essentially of sterile rhPDGF-AB in a physiologic solution and a sterile biocompatible carrier. In some embodiments, the composition comprises or consists essentially of sterile rhPDGF-BB and rhPDGF-AB in a physiologic solution and a sterile biocompatible carrier.

In some embodiments, the cosmetic method of promoting hair follicle regeneration and/or hair growth in a subject as described herein comprises: (i) forming a composition comprising or consisting essentially of recombinant human PDGF-BB (rhPDGF-BB) and/or recombinant human PDGF-AB (rhPDGF-AB) in a physiologic solution; and (ii) applying the composition onto or into hair follicles and surrounding skin and/or an area of hair loss. In some embodiments, the rhPDGF-BB and/or rhPDGF-AB are sterile. In some embodiments, the composition comprises or consists essentially of rhPDGF-BB in a physiologic solution. In some embodiments, the composition comprises or consists essentially of rhPDGF-AB in a physiologic solution. In some embodiments, the composition comprises or consists essentially of rhPDGF-BB and rhPDGF-AB in a physiologic solution.

In some embodiments, the cosmetic method of promoting hair follicle regeneration and/or hair growth in a subject as described herein comprises: (i) forming a composition comprising or consisting essentially of recombinant human PDGF-BB (rhPDGF-BB) and/or recombinant human PDGF-AB (rhPDGF-AB) in a physiologic solution and a biocompatible carrier, wherein the biocompatible carrier is a hyaluronic acid, collagen, gelatin, alginate, polysaccharide, glycosaminoglycan, fibrin, a derivative thereof or a combination thereof; and (ii) applying the composition onto or into hair follicles and surrounding skin and/or an area of hair loss. In some embodiments, the rhPDGF-BB and/or rhPDGF-AB are sterile. In some embodiments, the biocompatible carrier is sterile. In some embodiments, the biocompatible carrier is hyaluronic acid. In some embodiments, the composition comprises or consists essentially of rhPDGF-BB in a physiologic solution and a biocompatible carrier. In some embodiments, the composition comprises or consists essentially of rhPDGF-AB in a physiologic solution and a biocompatible carrier. In some embodiments, the composition comprises or consists essentially of rhPDGF-BB and rhPDGF-AB in a physiologic solution and a biocompatible carrier.

The “physiologic solution” described herein for forming the composition of the invention may comprise the buffer described herein. In some embodiments, the physiologic solution comprises sodium acetate. In some embodiments, the physiologic solution comprises sodium chloride. In some embodiments, the physiologic solution comprises sodium acetate and sodium chloride. In some embodiments, the physiologic solution comprises sodium acetate, sodium chloride and water. In some embodiments, the physiologic solution comprises about 20 mM sodium acetate, and optionally water. In some embodiments, the physiologic solution comprises about 0.15 M sodium chloride, and optionally water. In some embodiments, the physiologic solution comprises about 20 mM sodium acetate, about 0.15 M sodium chloride, and optionally water. In some embodiments, the physiologic solution comprises about 20 mM sodium acetate, water and optionally about 0.15 M sodium chloride. The physiologic solution may comprise a pH value of 6.0+/−0.5 (i.e., 5.5-6.5).

The sterile rhPDGF-BB solution described herein for forming the composition of the invention may comprise rhPDGF-BB at a concentration of at least 40 μg/mL, 50 μg/mL, 60 μg/mL, 70 μg/mL, 80 μg/mL, 90 μg/mL, 100 μg/mL, 200 μg/mL, 300 μg/mL, 400 μg/mL, 500 μg/mL, 600 μg/mL, 700 μg/mL, 800 μg/mL, 900 μg/mL, and 1,000 μg/mL. The solution may comprise rhPDGF-BB at a concentration of between about 100 μg/mL and 500 μg/mL. The solution may comprise rhPDGF-BB at a concentration of between about 300 μg/mL and 1,000 μg/mL. The solution may comprise rhPDGF-BB at a concentration of between about 200 μg/mL and 400 μg/mL. The solution may comprise rhPDGF-BB at a concentration of about 300 μg/mL.

The sterile rhPDGF-AB solution described herein for forming the composition of the invention may comprise rhPDGF-AB at a concentration of at least 40 μg/mL, 50 μg/mL, 60 μg/mL, 70 μg/mL, 80 μg/mL, 90 μg/mL, 100 μg/mL, 200 μg/mL, 300 μg/mL, 400 μg/mL, 500 μg/mL, 600 μg/mL, 700 μg/mL, 800 μg/mL, 900 μg/mL, and 1,000 μg/mL. The solution may comprise rhPDGF-AB at a concentration of between about 100 μg/mL and 500 μg/mL. The solution may comprise rhPDGF-AB at a concentration of between about 300 μg/mL and 1,000 μg/mL. The solution may comprise rhPDGF-AB at a concentration of between about 200 μg/mL and 400 μg/mL. The solution may comprise rhPDGF-AB at a concentration of about 300 μg/mL.

In some embodiments, forming the composition may comprise combining or mixing a rhPDGF-BB solution (e.g. a sterile physiological solution of rhPDGF-BB) with a carrier as described, e.g. a hyaluronic acid gel or serum as described herein. In some embodiments, the rhPDGF-BB solution and carrier (e.g. hyaluronic acid serum/gel) are mixed at a ratio of 1:2. In some embodiments, the rhPDGF-BB solution and carrier (e.g. hyaluronic acid serum/gel) are mixed at a ratio of 1:3. In some embodiments, the rhPDGF-BB solution and carrier (e.g. hyaluronic acid serum/gel) are mixed at a ratio of 1:4. In some embodiments, the rhPDGF-BB solution and carrier (e.g. hyaluronic acid serum/gel) are mixed at a ratio of 1:5. In some embodiments, the rhPDGF-BB solution and carrier (e.g. hyaluronic acid serum/gel) are mixed at a ratio of 1:6. In some embodiments, the rhPDGF-BB solution and carrier (e.g. hyaluronic acid serum/gel) are mixed at a ratio of 1:7. In some embodiments, the rhPDGF-BB solution and carrier (e.g. hyaluronic acid serum/gel) are mixed at a ratio of 1:8. In some embodiments, the rhPDGF-BB solution and carrier (e.g. hyaluronic acid serum/gel) are mixed at a ratio of 1:8. In some embodiments, the rhPDGF-BB solution and carrier (e.g. hyaluronic acid serum/gel) are mixed at a ratio of 1:10. In some embodiments, the HA gel or serum described herein for forming the composition may be a HA serum or gel containing about 0.5% to 2% HA, such as about 0.75% to about 1.75%, about 0.75% to about 1.5%, or about 1% to about 1.25%. In some embodiments, the HA gel or serum described herein for forming the composition may be a HA serum or gel containing about 1% HA. In some embodiments, the HA gel or serum described herein for forming the composition may be a HA serum or gel containing about 1.25% HA.

In some embodiments, forming the composition may comprise combining or mixing a rhPDGF-BB solution (e.g. a sterile physiological solution of rhPDGF-BB) with a carrier as described, e.g. a hyaluronic acid gel or serum as described herein. In some embodiments, the rhPDGF-BB solution and carrier (e.g. hyaluronic acid serum/gel) are mixed at a ratio of 1:1. In some embodiments, the rhPDGF-BB solution and carrier (e.g. hyaluronic acid serum/gel) are mixed at a ratio of 2:1. In some embodiments, the rhPDGF-BB solution and carrier (e.g. hyaluronic acid serum/gel) are mixed at a ratio of 3:1. In some embodiments, the rhPDGF-BB solution and carrier (e.g. hyaluronic acid serum/gel) are mixed at a ratio of 4:1. In some embodiments, the rhPDGF-BB solution and carrier (e.g. hyaluronic acid serum/gel) are mixed at a ratio of 5:1. In some embodiments, the rhPDGF-BB solution and carrier (e.g. hyaluronic acid serum/gel) are mixed at a ratio of 6:1. In some embodiments, the rhPDGF-BB solution and carrier (e.g. hyaluronic acid serum/gel) are mixed at a ratio of 7:1. In some embodiments, the rhPDGF-BB solution and carrier (e.g. hyaluronic acid serum/gel) are mixed at a ratio of 8:1.

In some embodiments, the method may further comprise mixing the combination of rhPDGF-BB and/or rhPDGF-AB and carrier with sodium chloride and/or water. For example, the method may comprise (i) combining a solution of rhPDGF-BB and/or rhPDGF-AB (e.g. rhPDGF-BB and/or rhPDGF-AB in a buffer described herein e.g. a sodium acetate buffer) with a carrier (e.g. hyaluronic acid as a serum or gel) and (ii) mixing the rhPDGF-BB and/or rhPDGF-AB and carrier with sodium chloride and/or water. For example, the method may comprise (i) combining a solution of rhPDGF-BB in a 20 mM sodium acetate buffer with a hyaluronic acid serum or gel (as described herein); and (ii) mixing the rhPDGF-BB and hyaluronic acid with sodium chloride and/or water. The buffer and carrier may be as described herein. The final concentration of rhPDGF-BB in the composition is as described herein. For example, the composition may comprise between about 40 μg PDGF/mL and 80 μg PDGF/mL. such as about 75 μg PDGF/mL.

In an exemplary embodiment, 0.5 mL of rhPDGF-BB solution at 300 μg/mL and 1.5 mL of hyaluronic acid (HA) gel or serum, which itself is a blend of: (i) hyaluronic acid with a weight of between about 100,000 Da and 150,000 Da and (ii) hyaluronic acid with a weight of between about 750,000 Da and 900,000 Da, are mixed thoroughly to form an rhPDGF-BB based therapeutic composition. The resultant mixture contains 150 ug of PDGF in 2 mL of gel, that is 75 ug PDGF/mL, and can be applied to skin disruptions after micro-needling or other aesthetics procedure. In some embodiments, sodium chloride and/or water are added to the rhPDGF-BB therapeutic composition prior to applying the composition to the subject's skin. In a further exemplary embodiment, 0.5 mL of rhPDGF-BB solution at 300 ug/mL and 3 mL of hyaluronic acid (HA) gel or serum, which itself is a blend of: (i) hyaluronic acid with a weight of between 100,000 Da and 150,000 Da and (ii) hyaluronic acid with a weight of between 750,000 Da and 900,000 Da, are mixed to form an rhPDGF-BB composition. The resultant mixture contains 150 ug of PDGF in 3.5 mL of composition, that is 42.86 μg PDGF/mL, and can be applied to skin disruptions after micro-needling or other aesthetics procedure. In some embodiments, sodium chloride and/or water are added to the rhPDGF-BB therapeutic composition prior to applying the composition to the subject's skin.

The methods and uses described herein may comprise applying (i.e., injecting) the composition via one or more needles. In some embodiments, the composition for use described herein is administered by injection. In some embodiments, the composition for use described herein is administered by sub-dermal injection. In some embodiments, the composition for use described herein is administered by micro-needling, laser or other penetration of the skin. In some embodiments, the composition is applied by micro-injections. In some embodiments, the composition for use described herein is administered by microchannel microinjector. For example, the composition may be applied (i.e., injected) into or onto the disruption via one or more needles. For example, the composition may be applied (i.e., injected) into or onto to the subject's skin via one or more needle. For example, the composition may be applied (i.e., injected) onto or into hair follicles and the surrounding skin. For example, the composition may be applied (i.e., injected) onto or into an area of hair loss. The needle may be a single use needle. For example, a single use needle may be used to inject the composition into the site of a skin disruption. For example, a single use needle may be used to inject the composition into hair follicles and the surrounding skin. For example, a single use needle may be used to inject the composition into an area of hair loss. Suitable skin disruptions to inject with the composition include scars, such as a surgical scar or acne scar. Skin disruptions caused by aging, inflammation, radiation damage, ultraviolet damage or combinations thereof (such as hair loss, wrinkles, sagging, furrows, skin laxity, sunburn, sunspots, irregular pigmentation, rough texture, dryness, large pores) may also be suitable for injection. The composition described herein may be mixed with other aesthetic compositions and injected into the skin or skin disruption. For example, the composition may be mixed with a dermal filler and injected into the skin or skin disruption. Exemplary dermal fillers include calcium hydroxyapatite filler (e.g. Radiesse®) (mineralized dermal fillers for facial folds) which are injected or placed along the jaw line and/or cheekbones to give definition. Other exemplary fillers include fillers comprising poly-L-lactic acid (e.g. Sculptra®) and fillers that comprise crosslinked hyaluronic acid (e.g. Juvéderm®). In some embodiments, the one or more needles are comprised within a microchannel microinjector (such as an Aquagold® device). A microchannel microinjector device comprises multiple hollow needles that contain the composition and deliver it into the subject's skin or skin disruption. In an exemplary microinjector, there is a reservoir at the top of the device which can be used to deliver the composition into the hollow needles for application to the subject's skin. The composition can be injected into skin or a skin disruption on any area of the subject's body. For example, the composition may be injected into the skin on the subject's head, face, scalp, chest, neck, arms, hands, and/or legs. For example, the composition may be injected into the skin on the subject's face, neck and/or scalp.

In some embodiments of the invention, the needle or other penetration device is hollow and delivers the therapeutic or cosmetic composition comprising rhPDGF-BB and/or rhPDGF-AB with or without a carrier to the disruption during the procedure. The needles may be micro-needles used in micro-needling procedures discussed above or the needle of a single injection-ready syringe. In some embodiments, the therapeutic or cosmetic composition is manufactured and pre-formulated in a syringe that can be used in the treatment methods disclosed herein by the user without preparation, mixing, or filling of the syringe pre- or pen-procedure. It is contemplated that the syringe can include a needle or be used without a needle to deliver the therapeutic composition to the disruption. In an embodiment, the depth of penetration of the needle is between about 0.1 mm and 5 mm, and generally about 1.0 mm-2.0 mm. In another embodiment, the needle is between about 0.5 mm to 5 in. In another embodiment, the needle is a 5 prong 1.5 mL needle (32 gauge). In another embodiment, the needle is the length and gauge necessary to deliver the therapeutic composition to the disruption.

The methods and uses described herein may comprise applying the composition topically. For example, the composition may be applied topically onto the subject's skin, including hair follicles and surrounding skin and/or an area of hair loss. For example, the composition may be applied topically onto the skin disruption. For example, the composition may be applied topically onto or into the hair follicles and surrounding skin. For example, the composition may be applied topically onto or into an area of hair loss. In some embodiments, the composition may be applied topically onto an incision in the skin. In a further example, the composition may be topically onto a skin incision resulting from a surgical procedure. The surgical procedure may be a cosmetic surgery such as a facelift surgery. The composition may be applied topically during a surgical procedure. For example, the composition may be applied to the open surgical wound or skin flap when closing the incision (e.g. using staples, glue or sutures). The composition may be applied topically to the incision after a surgical procedure e.g. immediately after the incision is closed. The composition may be applied during and/or after the surgical procedure. For example, the composition may be applied when closing the incision (e.g. using staples, glue or sutures) and immediately after the incision is closed. The incision may be re-treated on a daily basis or periodically. For example, the incision may be retreated using the re-treatment timing schedules described herein. For example, treatment of an incision may be repeated 1-2 times a day for a duration of 2, 3, 4, 5, 6, 7, 8, 9, or 10 days. The duration of re-treatment may be adjusted according to the severity of the incision. For deeper wounds/incisions (e.g. after Mohs surgery), the composition may be re-applied 1-2 times a day for between 7 to 10 days after surgery. For more superficial incisions, the composition may be re-applied 1-2 times a day for between 4 to 5 days after surgery. In some embodiments, the composition may be applied topically to skin that has a disruption caused by aging, inflammation, radiation damage, ultraviolet damage, or combinations thereof. In some embodiments, the composition is applied to a surface of the damaged skin or connective tissue. In some embodiments, the therapeutic or cosmetic composition may be applied on a body part such as, but not limited to, face, scalp, and/or neck (e.g. for repair or rejuvenation). The composition can be applied topically to skin or a skin disruption on any area of the subject's body. For example, the composition may be topically to the subject's head, face, scalp, back, chest, stomach, neck, arms, hands, and/or legs. In some embodiments, the composition is applied before, during, and/or after an aesthetic procedure (such as micro-needling, micro-injections, micro-coring, dermal abrasion, dermaplaning, dermal filling, or laser skin penetration described herein) to repair and rejuvenate the skin or other disruption. In another example, the composition may be applied to these or other body parts for aesthetic utility. However, a person skilled in the art would understand that the presented embodiments are not limited to the above applications and can be implemented in any suitable application related to the medical industry. Topical applications typically involve applying around 0.5 ml to 2 ml of the composition.

Further, application of the therapeutic or cosmetic composition may include penetration of skin (e.g. intact skin) via mechanical or chemical intervention to assist with the delivery of the therapeutic or cosmetic composition onto or into the disruption or the subject's skin. For example, in the methods and uses described herein, the composition may be applied topically onto the subject's skin or skin disruption, and mechanical or chemical means are used to penetrate the skin to assist with delivery of the applied composition into the skin or skin disruption. In some embodiments, the composition for use described herein is administered by penetration of skin (e.g. intact skin) via mechanical or chemical intervention. In some embodiments of the invention, the mechanical or chemical intervention may include energy-based devices (such as those used to perform micro-needling, micro-injections, micro-coring and laser resurfacing), excipients, and/or degradation to aid in the delivery of the therapeutic or cosmetic composition across the stratum-corneum onto or into the disruption or the subject's skin and increases clinical effectiveness. Penetration may allow delivery of the composition into the dermis and/or epidermis. Such a delivery method for rhPDGF-BB and/or rhPDGF-AB-based compositions is not contemplated by the methods known in the art to treat bone and tissue inside the body. In some embodiments, the penetration of the skin occurs via performing micro-needling, micro-injections, micro-coring, derm-abrasion, dermaplaning or laser skin resurfacing using mechanical devices, lasers or scraping tools and applying the therapeutic or cosmetic composition to the skin (e.g. of the face, neck, and/or scalp), before (pre-), during (peri-) or after (post) the procedure using such tools. Lasers are also often used in aesthetics and include ablative and non-ablative lasers. Non-ablative laser treatments, such as fractionated erbium (Er) lasers, intense pulsed light lasers, Alexandrite Lasers, and neodymium (Nd) lasers, are commonly used for the treatment of fine lines, mild wrinkles, and newly acquired dyspigmentation, as well as to improve skin tone and texture. Non-ablative lasers leave the epidermis intact while heating target tissue to produce rejuvenating skin effects. Ablative laser treatments, such as carbon dioxide lasers, pulsed dye lasers (PDL), and erbium (Er) lasers, are commonly used for the treatment of deep lines and wrinkles, acne scars, severe discoloration (brown or red spots), dyspigmentation, textural skin challenges, and scaring. Ablative lasers can create channels in the skin somewhat similar to mechanical micro-needling, and can be used to remove the outer epidermal layer from the entire skin, while heating the water molecules in the dermis to trigger the healing response. In some embodiments, the laser that penetrates the skin or skin disruption is an ablative laser. In some embodiments, the laser that penetrates the skin or skin disruption is a carbon dioxide laser, a pulsed dye laser (PDL), or an erbium laser (e.g., erbium-doped yttrium aluminum garnet (Er:YAG) laser). Non-ablative laser treatment may also be used in combination with the treatment methods and uses described herein. In some embodiments, the composition may be applied to the subject's skin or skin disruption before, during and/or after a laser resurfacing treatment using a non-ablative laser. In some embodiments, the non-ablative laser treatment comprises Broadband Light (BBL) therapy or Intense Pulsed Light (IPL) therapy, which uses short blasts of light that penetrate the skin and work to rejuvenate the skin. In some embodiments, the mechanical device that penetrates the skin or skin disruption is a micro-needling pen. In some embodiments, the device is a thermo-mechanical ablative treatment, such as, Tixel®, which, in addition to ablative and non-ablative treatments, has an open channel mode that opens channels to help transfer the composition into and onto the skin. In some embodiments, the mechanical device that penetrates the skin or skin disruption is a micro-coring device. Dermaplaning (or dermablading) is a procedure that involves scraping off the upper epidermis layer of skin, which exfoliates the skin and removes dead skin cells and fine facial hair. In some embodiments, the scraping tool that penetrates the skin or skin disruption is a dermaplaning tool or a dermaplaning scalpel, such as a dermatome. In some embodiments, the mechanical device that penetrates the skin or skin disruption is a microchannel microinjector (e.g. Aquagold®) which uses hollow needles to penetrate the skin. When using this procedure, the composition may be contained within the hollow needles of the microinjector and applied into the skin or skin disruption via the needles. This is different to the procedures (such as micro-needling, micro-coring or laser penetration) which introduce channels or disruptions in the epidermis or dermis of the subject's skin which assist with delivery of the topically applied composition into the subject's skin. In some embodiments, several tools are used to penetrate the skin to assist with delivery of the composition. For example, both lasers and micro-needling may be applied to the subject's skin or skin disruption, and the composition may be applied before, during and/or after the aesthetic procedures are performed. For example, a device (e.g. Morpheus8) that delivers radiofrequency (RF) energy through microneedles can be used to penetrate the skin.

The methods and uses described herein may comprise applying the composition before, during and/or after an aesthetic procedure (e.g. a skin penetrating aesthetic procedure) is performed on the subject's skin. In some embodiments, the composition may be applied before an aesthetic procedure (e.g. a skin penetrating aesthetic procedure) is performed on the subject's skin. In some embodiments, the composition may be applied during an aesthetic procedure (e.g. a skin penetrating aesthetic procedure) performed on the subject's skin. In some embodiments, the composition may be applied after an aesthetic procedure (e.g. a skin penetrating aesthetic procedure) is performed on the subject's skin. In some embodiments, the composition may be applied before and during an aesthetic procedure (e.g. a skin penetrating aesthetic procedure) performed on the subject's skin. In some embodiments, the composition may be applied before and after an aesthetic procedure (e.g. a skin penetrating aesthetic procedure) is performed on the subject's skin. In some embodiments, the composition may be applied before, during and after an aesthetic procedure (e.g. a skin penetrating aesthetic procedure) is performed on the subject's skin. As used herein the term “skin penetrating aesthetic procedure” refers to a procedure that penetrates the skin using a mechanical device (e.g. a needle, such as a microneedle, a micro-injection, micro-coring needle or microchannel microinjector), a scaping tool (e.g. for dermal abrasion or dermaplaning), lasers (ablative or non-ablative laser), or thermo-mechanical ablation technology (TMA) (e.g. Tixel®). In some embodiments, the composition may be applied topically before, during and/or after micro-needling, micro-injection, micro-coring, microchannel microinjection, derm-abrasion, dermaplaning, dermal filling or laser skin resurfacing. When the composition is applied “before” an aesthetic procedure, this involves topically applying the composition before an aesthetic procedure is performed. In some embodiments, this involves applying the composition at least 5 mins, 15 minutes, 20 minutes, 25 minutes 30 minutes, 1 hour, 2 hours, 3 hours, 4 hours, 5 hours, 6 hours, 12 hours, 24 hours, 2 days, 3 days, 4 days, 5 days, or 6 days before an aesthetic procedure is performed. For example, the composition may be applied immediately before the procedure (e.g. 5-10 minutes before). When the composition is applied “during” an aesthetic procedure, this involves topically applying the composition throughout the procedure (e.g. in a single treatment appointment between application of the skin penetration steps). For example, the aesthetic procedure (e.g. micro-needling, micro-injection, micro-coring or laser penetration) may be performed by dividing the subject's skin into various subareas (or “quadrants”) and each subarea may be subjected to the aesthetic procedure (e.g. using vertical, horizontal, and diagonal movements across the same area of the skin or disruption being treated). The procedure may be repeated up to 3 times on the same subarea in a single treatment appointment. The composition may be re-applied (e.g. topically) between each repeat of the procedure on the subarea (or quadrant) of the subject's skin. When the composition is applied “after” an aesthetic procedure, this involves topically applying the composition after an aesthetic procedure is performed. In some embodiments, this involves applying the composition within 5 minutes, 10 minutes, 15 minutes, 20 minutes, 25 minutes, 30 minutes, 1 hour, 2 hours, 3 hours, 4 hours, 5 hours, 6 hours, 12 hours, or 24 hours of an aesthetic procedure being performed. In some embodiments, the composition can be applied up to 2 days, 3 days, 4 days, 5 days, 6 days, 7 days, 8 days, 9 days, 10, days, 11 days, 12 days, 13 days, or 14 days after an aesthetic procedure is performed. For example, the composition may be applied immediately after the aesthetic procedure e.g. within 5-10 minutes. The composition may be periodically re-applied to the subject's skin according to the retreatment timing schedule described herein. For example, the subject's skin can be re-treated by applying the composition 1-2 times a day for up to 14 days. For example, the subject's skin can be re-treated by applying the composition 2-3 times a day for up to 14 days. In some embodiments, the composition is applied (e.g. topically) before, during and/or after micro-needling. In some embodiments, the composition is applied (e.g. topically) before micro-needling. In some embodiments, the composition is applied (e.g. topically) during micro-needling (i.e., between repeat penetration steps of the micro-needling procedure). In some embodiments, the composition is applied (e.g. topically) after micro-needling. In some embodiments, the composition is applied (e.g. topically) before, during and/or after micro-injection. In some embodiments, the composition is applied (e.g. topically) before micro-injection. In some embodiments, the composition is applied (e.g. topically) during micro-injection (i.e., between repeat penetration steps of the micro-injection procedure). In some embodiments, the composition is applied (e.g. topically) after micro-injection. In some embodiments, the composition is applied (e.g. topically) before, during and/or after a laser resurfacing treatment. In some embodiments, the composition is applied (e.g. topically) before a laser resurfacing treatment. In some embodiments, the composition is applied (e.g. topically) during a laser resurfacing treatment. In some embodiments, the composition is applied (e.g. topically) after a laser resurfacing treatment. In some embodiments, the composition is applied (e.g. topically) before, during, and/or after TMA. These aesthetic procedures penetrate the subject's skin and assist with delivering the PDGF into or onto the skin's surface. When the composition is applied before, during and/or after the aesthetic procedure, the composition may assist with promoting skin and/or hair follicle regeneration, rejuvenation or repair and/or hair growth. When the composition is applied before, during and/or after the aesthetic procedure, the composition may assist with preventing or reducing pain or discomfort; or with preventing or reducing redness, swelling and inflammation.

Any skin disruption described herein can be suitably treated using the composition of the invention, which is applied before, during and/or after an aesthetic procedure. For example, a scar (such as a surgical scar or acne scar) can be treated using the methods and uses described herein which involve applying the composition before, during and/or after an aesthetic procedure, such as micro-needling, micro-injection, micro-coring, dermal abrasion, skin resurfacing, dermaplaning or laser treatment. For example, a scar may be treated using the composition described herein which is applied before a micro-needling procedure is performed. For example, a scar may be treated using the composition described herein which is applied after a laser resurfacing treatment is performed. Skin disruptions that are caused by aging, inflammation, radiation damage, ultraviolet damage or combinations thereof (such as wrinkles, sagging, furrows, skin laxity, sunburn, sunspots, sunburn, irregular pigmentation, rough texture, dryness, large pores) may also be treated using the methods and uses described herein which involve applying the composition described herein before, during and/or after an aesthetic procedure, such as micro-needling, micro-injection, micro-coring, dermal abrasion, dermaplaning, skin resurfacing, or laser treatment. For example, skin disruptions caused by aging thereof (such as hair loss, wrinkles, sagging, furrows, skin laxity, rough texture, dryness, large pores) may be treated using the composition described herein which is applied before a micro-needling procedure is performed. For example, skin disruptions caused by aging (such as wrinkles, sagging, furrows, skin laxity, rough texture, dryness, large pores) may be treated using the composition described herein which is applied after a micro-needling procedure is performed. For example, skin disruptions caused by aging (such as hair loss, wrinkles, sagging, furrows, skin laxity, rough texture, dryness, large pores) may be treated using the composition described herein which is applied before a laser resurfacing treatment is performed. For example, skin disruptions caused by aging (such as wrinkles, sagging, furrows, skin laxity, rough texture, dryness, large pores) may be treated using the composition described herein which is applied after a laser resurfacing treatment is performed. For example, skin disruptions caused by hyperpigmentation or melasma may be treated using the composition described herein which is applied before a micro-needling procedure is performed. For example, skin disruptions caused by hyperpigmentation or melasma may be treated using the composition described herein which is applied after a micro-needling procedure is performed. For example, skin disruptions caused by hyperpigmentation or melasma may be treated using the composition which is applied before a laser resurfacing treatment is performed. For example, skin disruptions caused by hyperpigmentation or melasma may be treated using the composition which is applied after a laser resurfacing treatment is performed.

Any hair follicles and surrounding skin and/or area of hair loss described herein can be suitably treated using the methods and uses described herein which involve applying the composition before, during and/or after an aesthetic procedure, such as micro-needling, micro-injection, micro-coring, dermal abrasion, skin resurfacing, dermaplaning or laser treatment. For example, hair follicles and surrounding skin can be treated using the methods and uses described herein which involve applying the composition before, during and/or after an aesthetic procedure, such as micro-needling, micro-injection, micro-coring, dermal abrasion, skin resurfacing, dermaplaning or laser treatment. For example, hair follicles and surrounding skin may be treated using the composition described herein which is applied before a micro-needling procedure is performed. For example, hair follicles and surrounding skin may be treated using the composition described herein which is applied before a micro-injection procedure is performed. For example, hair follicles and surrounding skin may be treated using the composition described herein which is applied after a laser resurfacing treatment is performed. In some embodiments, the hair follicles and surrounding skin are healthy. In some embodiments, the hair follicles and surrounding skin are on a subject having reduced total hair count, hair density, mean thickness, and/or cumulative hair thickness, in comparison to measurements taken from the subject at a previous time or average measurements. In some embodiments, the reduced total hair count, hair density, mean thickness, and/or cumulative hair thickness is caused by aging. In some embodiments, the reduced total hair count, hair density, mean thickness, and/or cumulative hair thickness is caused by inflammation, radiation damage, ultraviolet damage, or combinations thereof. In some embodiments, the reduced total hair count, hair density, mean thickness, and/or cumulative hair thickness is caused by an incision from a surgical procedure or other medical intervention, such as chemotherapy, radiation, or medication. In some embodiments, the reduced total hair count, hair density, mean thickness, and/or cumulative hair thickness is caused by scarring. In some embodiments, the reduced total hair count, hair density, mean thickness, and/or cumulative hair thickness is caused by an aesthetic procedure, as described herein, such as micro-needling, micro-injection, micro-coring, dermaplaning, dermal abrasion, dermal filling or laser resurfacing. An area of hair loss can be treated using the methods and uses described herein which involve applying the composition before, during and/or after an aesthetic procedure, such as micro-needling, micro-injection, micro-coring, dermal abrasion, skin resurfacing, dermaplaning or laser treatment. For example, an area of hair loss may be treated using the composition described herein which is applied before a micro-needling procedure is performed. For example, an area of hair loss may be treated using the composition described herein which is applied before a micro-injection procedure is performed. For example, an area of hair loss may be treated using the composition described herein which is applied after a laser resurfacing treatment is performed. In some embodiments, the area of hair loss is on healthy skin. In some embodiments, the area of hair loss is caused by ageing. In some embodiments, the area of hair loss is caused by inflammation, radiation damage, ultraviolet damage, or combinations thereof. In some embodiments, the area of hair loss is caused by an incision from a surgical procedure or other medical intervention, such as chemotherapy, radiation, or medication. In some embodiments, the area of hair loss is due to a scar. In some embodiments, the area of hair loss is caused by an aesthetic procedure, as described herein, such as micro-needling, micro-injection, micro-coring, dermaplaning, dermal abrasion, dermal filling or laser resurfacing.

In some embodiments, the invention provides a method (e.g. cosmetic method) of treating a skin disruption caused by aging, inflammation, radiation damage, ultraviolet damage or combinations thereof (such as hair loss, wrinkles, sagging, furrows, sunburn, sunspots, irregular pigmentation, rough texture and dryness) in a subject, wherein said method comprises: (1) providing a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier comprising hyaluronic acid; and (2) applying the composition (e.g. topically) onto the skin disruption before, during and/or after a skin penetrating aesthetic procedure is performed (e.g. on the same area of the subject's skin) (such as micro-needling, micro-injection, micro-coring, dermal abrasion or dermal filling), wherein the carrier delivers biologically active rhPDGF onto or into the disruption to regenerate, rejuvenate or repair the subject's skin. Advantageously, the skin penetrating aesthetic procedure penetrates the subject's skin to facilitate delivery of the composition to the skin disruption. In some embodiments, the skin disruption does not involve tendons, ligaments or bones. In some embodiments, the skin disruption extends only into the dermal or subdermal layers of the subject's skin. In some embodiments, the composition comprises or consists essentially of sterile rhPDGF-BB in a physiologic solution and a sterile biocompatible carrier comprising hyaluronic acid. In some embodiments, the composition comprises or consists essentially of sterile rhPDGF-AB in a physiologic solution and a sterile biocompatible carrier comprising hyaluronic acid. In some embodiments, the composition comprises or consists essentially of sterile rhPDGF-BB and rhPDGF-AB in a physiologic solution and a sterile biocompatible carrier comprising hyaluronic acid.

In some embodiments, the invention provides a method (e.g. cosmetic method) of treating a skin disruption caused by aging, inflammation, radiation damage, ultraviolet damage or combinations thereof (such as hair loss, wrinkles, sagging, furrows, sunburn, sunspots, irregular pigmentation, rough texture and dryness) in a subject, wherein said method comprises: (1) providing a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier comprising hyaluronic acid; and (2) applying the composition (e.g. topically) onto the skin disruption before, during and/or after a micro-needling procedure is performed (e.g. on the same area of the subject's skin), wherein the carrier delivers biologically active rhPDGF onto or into the disruption to regenerate, rejuvenate or repair the subject's skin. Advantageously, the micro-needling procedure penetrates the subject's skin to facilitate delivery of the composition to the skin disruption. In some embodiments, the skin disruption does not involve tendons, ligaments or bones. In some embodiments, the skin disruption extends only into the dermal or subdermal layers of the subject's skin. In some embodiments, the composition comprises or consists essentially of sterile rhPDGF-BB in a physiologic solution and a sterile biocompatible carrier comprising hyaluronic acid. In some embodiments, the composition comprises or consists essentially of sterile rhPDGF-AB in a physiologic solution and a sterile biocompatible carrier comprising hyaluronic acid. In some embodiments, the composition comprises or consists essentially of sterile rhPDGF-BB and rhPDGF-AB in a physiologic solution and a sterile biocompatible carrier comprising hyaluronic acid.

In some embodiments, the invention provides a method (e.g. cosmetic method) of treating a skin disruption caused by aging, inflammation, radiation damage, ultraviolet damage or combinations thereof (such as hair loss, wrinkles, sagging, furrows, sunburn, sunspots, irregular pigmentation, rough texture and dryness) in a subject, wherein said method comprises: (1) providing a composition comprising or consisting essentially of sterile recombinant human PDGF-BB (rhPDGF-BB) and/or sterile recombinant human PDGF-AB (rhPDGF-AB) in a physiologic solution and a sterile biocompatible carrier comprising hyaluronic acid; and (2) applying the composition (e.g. topically) onto the skin disruption after a micro-needling procedure has been performed (e.g. on the same area of the subject's skin), wherein the carrier delivers biologically active rhPDGF onto or into the disruption to regenerate, rejuvenate or repair the subject's skin. Advantageously, the micro-needling procedure penetrates the subject's skin to facilitate delivery of the composition to the skin disruption. In some embodiments, the skin disruption does not involve tendons, ligaments or bones. In some embodiments, the skin disruption extends only into the dermal or subdermal layers of the subject's skin. In some embodiments, the composition comprises or consists essentially of sterile rhPDGF-BB in a physiologic solution and a sterile biocompatible carrier comprising hyaluronic acid. In some embodiments, the composition comprises or consists essentially of sterile rhPDGF-AB in a physiologic solution and a sterile biocompatible carrier comprising hyaluronic acid. In some embodiments, the composition comprises or consists essentially of sterile rhPDGF-BB and rhPDGF-AB in a physiologic solution and a sterile biocompatible carrier comprising hyaluronic acid.

As described herein, application of the composition may include penetration or perforation of skin (e.g. intact skin), hair follicles and surrounding skin, and/or an area of hair loss via mechanical or chemical intervention to assist with the delivery of the composition onto or into the disruption or the subject's skin. In some embodiments, the composition for use described herein is administered by penetration or perforation of skin (e.g. intact skin), hair follicles and surrounding skin, and/or an area of hair loss via mechanical or chemical intervention to assist with the delivery of the composition onto or into the disruption or the subject's skin. Further, application of the therapeutic or cosmetic composition may include performing micro-needling or micro-injection or micro-coring or derm-abrasion using mechanical devices, lasers, scraping, or other resurfacing tools and applying the therapeutic composition to the skin of the face, neck, and/or scalp, before, during and/or after the use of such tools. In some embodiments, the depth of penetration is between about 0.2 mm and 0.5 mm, 0.2 mm and 3 mm, or 0.5 mm and 3 mm depending on the thickness of the skin. For example, the depth of penetration may be between about 0.2 mm and 0.5 mm. For example, the depth of penetration may be between about 0.5 mm to 1.0 mm. For example, the depth of penetration may be between about 1.0 mm and 2.0 mm. In some embodiments, the depth of penetration of the needles or lasers is between about 0.2 mm and 0.5 mm, or between about 0.5 mm and 3 mm, and generally about 0.5 mm to 1.0 mm or 1.0 mm-2.0 mm depending on the thickness of the skin. For example, the depth of penetration of the needles or lasers may be between about 0.2 mm and 0.5 mm. For example, the depth of penetration of the needles or lasers may be between about 0.2 mm to 1.0 mm. For example, the depth of penetration of the needles or lasers may be between about 0.5 mm to 1.0 mm. For example, the depth of penetration of the needles or lasers may be between about 1.0 mm and 2.0 mm. Some procedures described herein penetrate the skin more deeply. For example, Morpheus8 laser treatment typically penetrates between 4 mm and 8 mm. Thus, in some embodiments, the depth of penetration of the needles or laser may be between 4 mm and 8 mm. In some embodiments of the methods and uses disclosed herein, the action of skin penetration via micro-needling, micro-injection, or micro-coring delivers the composition (e.g. the composition of rhPDGF-BB and hyaluronic acid or other carrier, the composition of rhPDGF-AB and hyaluronic acid or other carrier, or the composition of rhPDGF-BB, rhPDGF-AB, and hyaluronic acid or other carrier) into the subepithelial dermal tissues of the skin of the body part being treated. In an exemplary embodiment, the hyaluronic is a serum or gel. In some embodiments, the hyaluronic acid is applied to the disruption prior to the penetration of the disruption via micro-needling, micro-injection, micro-coring or other procedure. In an embodiment, the hyaluronic is a serum or gel. In some embodiments, the hyaluronic acid has a pH value of 5.0 to 7.0. The hyaluronic acid may have the wt % described herein.

In some embodiments, the micro-needling, micro-injection, laser or other penetration of the skin is repeated up to 6 times. For example, the skin penetration may be repeated up to 2 times, up to 3 times, up to 4 times, up to 5 times or up to 6 times. In some embodiments of the invention, the micro-needling is repeated three times, such as in vertical, horizontal, and diagonal movements across the same area of the skin or other disruption being treated. It is also contemplated that the penetration of the same area of the skin is repeated with movement in the same direction. In another aspect of the invention, the area of the skin being treated is separated into subareas, and the initial penetration of the skin and repeated penetrations are conducted in each subarea. In one embodiment, the subarea is a 1.5 square inch area of the skin, but any size subarea is contemplated herein. In some embodiments, the composition may be reapplied topically between each repeat penetration.

In some embodiments, the (therapeutic or cosmetic) composition remains on the skin or other disruption, hair follicle and surrounding skin, and/or area of hair loss post application for at least 24 hours. In some embodiments, the (therapeutic or cosmetic) composition remains on the skin or other disruption, hair follicle and surrounding skin, and/or area of hair loss for up to 48 hours. After the minimal 24-hour period, the user can cleanse the treated area with water and reapply the composition or hyaluronic acid.

In some embodiments, the (therapeutic or cosmetic) composition (e.g. the composition of rhPDGF-BB and hyaluronic acid or other carrier, the composition of rhPDGF-AB and hyaluronic acid or other carrier, or the composition of rhPDGF-BB, rhPDGF-AB, and hyaluronic acid or other carrier) is applied topically to the skin before, during, or after TMA. In some embodiments, this treatment is repeated. In some embodiments, repeat treatment comprises the topical application of the PDGF only composition to the disruption. In some embodiments, the (therapeutic or cosmetic) composition (e.g. the composition of rhPDGF-BB and hyaluronic acid or other carrier, the composition of rhPDGF-AB and hyaluronic acid or other carrier, or the composition of rhPDGF-BB, rhPDGF-AB, and hyaluronic acid or other carrier) is applied topically to the skin after TMA and repeat treatment comprises the topical application of the PDGF only composition to the disruption. In some embodiments, the (therapeutic or cosmetic) composition (e.g. the composition of rhPDGF-BB and hyaluronic acid or other carrier, the composition of rhPDGF-AB and hyaluronic acid or other carrier, or the composition of rhPDGF-BB, rhPDGF-AB, and hyaluronic acid or other carrier) is applied topically to the skin during TMA and repeat treatment comprises the topical application of the PDGF only composition to the disruption. In some embodiments, the (therapeutic or cosmetic) composition (e.g. the composition of rhPDGF-BB and hyaluronic acid or other carrier, the composition of rhPDGF-AB and hyaluronic acid or other carrier, or the composition of rhPDGF-BB, rhPDGF-AB, and hyaluronic acid or other carrier) is applied topically to the skin before TMA and repeat treatment comprises the topical application of the PDGF only composition to the disruption. In some embodiments, the (therapeutic or cosmetic) composition (e.g. the composition of rhPDGF-BB and hyaluronic acid or other carrier, the composition of rhPDGF-AB and hyaluronic acid or other carrier, or the composition of rhPDGF-BB, rhPDGF-AB, and hyaluronic acid or other carrier) is applied topically to the skin before and during TMA and repeat treatment comprises the topical application of the PDGF only composition to the disruption.

As used herein, the “user” may include a dermatologist, plastic surgeon, physician, esthetician, the human being receiving the treatment or other individual qualified to perform the relevant methods to treat the disruption on the human being disclosed herein. Thus, in some embodiments of the invention, the patient (or subject) may complete the method and treat and/or retreat his or her own disruption. In another example, there may be two or more different users of the method over the course of the treatment and retreatments described herein. For example, a patient (or subject) may have a treatment completed in a dermatologists or estheticians office and apply the retreatment in the comfort of his or her home.

In some embodiments of the invention described herein, the therapeutic or cosmetic composition is added to compositions known in the art to repair and rejuvenate skin and other disruptions in aesthetic procedures, such as dermal and body contouring fillers, during the manufacture process of the known composition, and the resultant composition is applied to the skin disruption pre-, peri-, or post aesthetic procedure. Unlike most proteins which are inactivated during heated sterilization techniques, rhPDGF-BB and rhPDGF-AB have heat resistant properties that allow them to survive the thermal effects of moist heat sterilization better than other proteins. These heat-resistant properties may allow the rhPDGF-BB and rhPDGF-AB to be added to existing manufacturing processes for use in aesthetic compositions and methods of treatment.

The methods and uses described herein may further comprise a monitoring step in which the efficacy of the treatment is monitored during the treatment period and the treatment step (“step 2”) of the method is repeated, if required. For example, the method for promoting skin regeneration, rejuvenation or repair described herein may further comprise monitoring regeneration, rejuvenation or repair of the subject's skin during a treatment period and repeating step (2) of the method to retreat the disruption. For example, the method for treating a skin disruption described herein may further comprise monitoring regeneration, rejuvenation or repair of the skin disruption during a treatment period and repeating step (2) of the method to retreat the disruption. For example, the method for preventing or reducing pain or discomfort or the method of improving pain or discomfort caused by the skin disruption described herein may further comprise monitoring the pain or discomfort during a treatment period and repeating step (2) of the method to retreat the disruption. For example, the method for preventing or reducing redness, swelling and/or inflammation caused by a skin disruption described herein may further comprise monitoring the redness, swelling and/or inflammation during a treatment period and repeating step (2) of the method to retreat the disruption. For example, the method for promoting hair follicle regeneration and/or hair growth described herein may further comprise monitoring the hair follicle regeneration and/or hair growth during a treatment period and repeating step (2) of the method to retreat the hair follicles and surrounding skin and/or area of hair loss.

For effective results, the above-described procedure may be repeated about weekly or monthly or semi-annually or annually for up to 6 instances. In an embodiment of the invention, the composition is reapplied 1 to 2 times per day for up to 14 days. For example, the composition may be reapplied 1 to 2 times for day for up to 2 days, 3 days, 4 days, 5 days, 6 days, 7 days, 8 days, 9 days, 10 days, 11 days, 12 days, 13 days or 14 days. In some embodiments, the composition may be reapplied 2 to 3 times per day for up to 14 days (e.g. up to 2, 3, 4, 5, 6, 7, 8, 9, 10, 11, 12, 13 or 14 days). In some embodiment of the invention, the composition is reapplied 2 to 3 times per day for up to 14 days.

In some embodiments of the invention, there is provided a timing schedule for periodically retreating the disruption, i.e., periodically reapplying the (therapeutic or cosmetic) composition to the disruption. In some embodiments of the invention, there is provided a timing schedule for periodically retreating the hair follicles and surrounding skin and/or area of hair loss i.e., periodically reapplying the cosmetic composition to the hair follicles and surrounding skin and/or area of hair loss. The actual number of retreatments and the retreating frequency (i.e., the treatment interval) should be determined based on a number of factors including the severity of the disruption, the extent to which the natural disruption healing environment is compromised, including the method for creating the disruption, patient's age, and duration of the disruption. The number of retreatments and the retreatment frequency should be increased for more severe disruptions or for disruptions with more compromised healing environments. In addition, the prescribed number of treatments and/or the retreatment frequency may be adjusted during the treatment period based on the disruption's rate of healing, i.e., increase number of retreatments and/or retreatment frequency for slower healing disruptions, or decrease number of retreatments and/or retreatment frequency for faster healing disruptions.

In some embodiments of the invention, the retreatment frequency is at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days and so on up to at least about once every six weeks, or combinations thereof. In some embodiments of the invention, the retreatment frequency is at least within about 2 days, at least within about 3 days, at least within about 4 days, at least within about 5 days, at least within about 6 days, at least within about 7 days, at least within about 8 days, at least within about 9 days, at least within about 10 days, at least within about 11 days, at least within about 12 days, at least within about 13 days, at least within about 14 days, or at least within about 15 days and so on up to at least about within six weeks of the first treatment, or combinations thereof. In some embodiments, the retreatment frequency is once every 2 to 42 days, or once every 3 to 42 days, or once every 2 to 28 days, or once every 3 to 28 days, or once every 2 to 7 days, or once every 2 to 3 days, or once every 3 to 7 days, or once every 4 to 21 days, once every 7 to 28 days, or once every 7 to 21 days, or once every 7 to 14 days, or once every 10 to 15 days, or once every 12 to 14 days. In some embodiments, the retreatment frequency is once every 2 days, 3 days, 4 days, 5 days, 6 days, 7 days, 8 days, 9 days, 10 days, 12 days, 14 days, 15 days, 21 days, 28 days, 30 days, 35 days, 42 days, or combinations thereof. In another aspect of the invention, the retreatment frequency of the above listed days is more than once per day, such as one to two times per day or two to three times per day.

In some embodiments, the retreatment frequency is substantially the same over the treatment period, and the retreatment frequency is one time at least about every 2 days, at least about every 3 days, at least about every 4 days, at least about every 5 days, at least about every 6 days, at least about every 7 days, at least about every 8 days, at least about 9 every days, at least about every 10 days, at least about every 11 days, at least about every 12 days, at least about every 13 days, at least about every 14 days, or at least about every 15 days and so on up to at least about once per year.

In some embodiments, the disruption, hair follicle and surrounding skin, and/or area of hair loss is retreated at least 1 time, at least 2 times, at least 3 times, at least 4 times, or at least 5 times over the treatment, or at least 6 times over the treatment period. In accordance with another aspect of the invention, the disruption, hair follicle and surrounding skin, and/or area of hair loss is retreated between 0 and 6 times, between 0 and 7 times, or between 0 and 8 times over the treatment period. In accordance with another aspect of the invention, the disruption, hair follicle and surrounding skin, and/or area of hair loss is treated between 1 to 8 times, or between 2 to 7 times, or between 3 to 6 times over the treatment period. In accordance with another aspect of the invention, the disruption, hair follicle and surrounding skin, and/or area of hair loss is repeated 1, 2, 3, 4, 5, 6, 7, 8, 10, or 20 times over the treatment period. In accordance with another aspect of the invention the disruption, hair follicle and surrounding skin, and/or area of hair loss is retreated between 0 and 46 times, or between 1 and 46 times, or between 0 and 20 times, or between 1 and 20 times, or between 0 and 27 times, or between 1 and 27 times.

In some embodiments, the cumulative total amount of rhPDGF-BB applied to the disruption during the treatment period is preferably more than 0 mg, but less than about 100 mg, 50 mg, or less than about 25 mg, or less than about 20 mg, or less than about 15 mg, or less than about 10 mg, or less than about 5 mg, or less than about 4 mg, or less than about 3 mg, or less than about 2 mg, or less than about 1 mg of rhPDGF-BB. In some embodiments, the cumulative total amount of rhPDGF-BB applied to the disruption during the treatment period is preferably between about 0.1 mg to about 50 mg, or about 0.5 mg to about 25 mg, or about 1 mg to about 10 mg, or about 2.5 mg to about 8 mg, or about 3 mg to about 7 mg, about 4 mg to about 6 mg. In some embodiments, the cumulative total amount of rhPDGF-BB applied to the disruption during the treatment period is between about 0.05 mg to about 0.1 mg, such as about 0.06 mg to about 0.1 mg, about 0.07 mg to about 0.09 mg, or about 0.07 mg to about 0.08 mg. In some embodiments, the cumulative total amount of rhPDGF-BB applied to the disruption during the treatment period is between about 0.1 mg to about 1 mg, such as about 0.2 mg to about 1 mg, about 0.3 mg to about 1 mg, about 0.4 mg to about 1 mg, about 0.5 mg to about 1 mg, about 0.6 mg to about 1 mg, about 0.7 mg to about 1 mg, about 0.8 mg to about 1 mg or about 0.9 mg to about 1 mg. In some embodiments, the cumulative total amount of rhPDGF-BB applied to the disruption during the treatment period is about 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg. 1.6 mg, 1.7 mg, 1.8 mg, 1.9 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, or 100 mg.

In some embodiments, the cumulative total amount of rhPDGF-AB applied to the disruption during the treatment period is preferably more than 0 mg, but less than about 100 mg, 50 mg, or less than about 25 mg, or less than about 20 mg, or less than about 15 mg, or less than about 10 mg, or less than about 5 mg, or less than about 4 mg, or less than about 3 mg, or less than about 2 mg, or less than about 1 mg of rhPDGF-AB. In some embodiments, the cumulative total amount of rhPDGF-AB applied to the disruption during the treatment period is preferably between about 0.1 mg to about 50 mg, or about 0.5 mg to about 25 mg, or about 1 mg to about 10 mg, or about 2.5 mg to about 8 mg, or about 3 mg to about 7 mg, about 4 mg to about 6 mg. In some embodiments, the cumulative total amount of rhPDGF-AB applied to the disruption during the treatment period is between about 0.05 mg to about 0.1 mg, such as about 0.06 mg to about 0.1 mg, about 0.07 mg to about 0.09 mg, or about 0.07 mg to about 0.08 mg. In some embodiments, the cumulative total amount of rhPDGF-AB applied to the disruption during the treatment period is between about 0.1 mg to about 1 mg, such as about 0.2 mg to about 1 mg, about 0.3 mg to about 1 mg, about 0.4 mg to about 1 mg, about 0.5 mg to about 1 mg, about 0.6 mg to about 1 mg, about 0.7 mg to about 1 mg, about 0.8 mg to about 1 mg or about 0.9 mg to about 1 mg. In some embodiments, the cumulative total amount of rhPDGF-AB applied to the disruption during the treatment period is about 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg. 1.6 mg, 1.7 mg, 1.8 mg, 1.9 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, or 100 mg.

In some embodiments, the cumulative total amount of rhPDGF-BB and rhPDGF-AB applied to the disruption during the treatment period is preferably more than 0 mg, but less than about 100 mg, 50 mg, or less than about 25 mg, or less than about 20 mg, or less than about 15 mg, or less than about 10 mg, or less than about 5 mg, or less than about 4 mg, or less than about 3 mg, or less than about 2 mg, or less than about 1 mg of rhPDGF-BB and rhPDGF-AB. In some embodiments, the cumulative total amount of rhPDGF-BB and rhPDGF-AB applied to the disruption during the treatment period is preferably between about 0.1 mg to about 50 mg, or about 0.5 mg to about 25 mg, or about 1 mg to about 10 mg, or about 2.5 mg to about 8 mg, or about 3 mg to about 7 mg, about 4 mg to about 6 mg. In some embodiments, the cumulative total amount of rhPDGF-BB and rhPDGF-AB applied to the disruption during the treatment period is between about 0.05 mg to about 0.1 mg, such as about 0.06 mg to about 0.1 mg, about 0.07 mg to about 0.09 mg, or about 0.07 mg to about 0.08 mg. In some embodiments, the cumulative total amount of rhPDGF-BB and rhPDGF-AB applied to the disruption during the treatment period is between about 0.1 mg to about 1 mg, such as about 0.2 mg to about 1 mg, about 0.3 mg to about 1 mg, about 0.4 mg to about 1 mg, about 0.5 mg to about 1 mg, about 0.6 mg to about 1 mg, about 0.7 mg to about 1 mg, about 0.8 mg to about 1 mg or about 0.9 mg to about 1 mg. In some embodiments, the cumulative total amount of rhPDGF-BB and rhPDGF-AB applied to the disruption during the treatment period is about 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg. 1.6 mg, 1.7 mg, 1.8 mg, 1.9 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, or 100 mg.

The various retreatments may involve the same or different dosages of rhPDGF-BB and/or rhPDGF-AB, either in terms of the exact amount of rhPDGF-BB and/or rhPDGF-AB that is applied to the disruption, hair follicles and surrounding skin, and/or an area of hair loss (i.e., “absolute dosage”) or in terms of the amount of rhPDGF-BB and/or rhPDGF-AB that is applied per square centimeter (cm²) of skin or disruption area, hair follicles and surrounding skin, and/or an area of hair loss (disruption area) (i.e., “area dosage”). In some embodiments, each treatment applies an absolute dosage of between about 10 μg and about 50 mg, or between about 10 μg and about 25 mg, or between about 10 μg and about 20 mg, or between about 10 μg and about 15 mg, or between about 10 μg and about 10 mg, or between about 10 μg and about 5 mg of rhPDGF-BB or between about 10 μg and about 1 mg or rhPDGF-BB. In some embodiments, each treatment applies an absolute dosage of between about 10 μg and about 50 mg, or between about 10 μg and about 25 mg, or between about 10 μg and about 20 mg, or between about 10 μg and about 15 mg, or between about 10 μg and about 10 mg, or between about 10 μg and about 5 mg of rhPDGF-AB or between about 10 μg and about 1 mg or rhPDGF-AB. In some embodiments, each treatment applies an absolute dosage of between about 10 μg and about 50 mg, or between about 10 μg and about 25 mg, or between about 10 μg and about 20 mg, or between about 10 μg and about 15 mg, or between about 10 μg and about 10 mg, or between about 10 μg and about 5 mg of rhPDGF-BB and rhPDGF-AB or between about 10 μg and about 1 mg or rhPDGF-BB and rhPDGF-AB. In some embodiments, each treatment applies an area dosage between about 10 μg PDGF/cm² and about 1.0 mg PDGF/cm², or between about 10 μg PDGF/cm² and about 0.5 mg PDGF/cm², or between about 10 μg PDGF/cm² and about 0.25 mg PDGF/cm², or between about 10 μg PDGF/cm² and 0.1 mg PDGF/cm², or between about 10 μg PDGF/cm² and about 0.05 mg PDGF/cm². In some embodiments, each treatment with rhPDGF-BB is preferably between about 10 μg to 1000 μg PDGF/cm², or about 0.01 mg to about 50 mg PDGF/cm², or about 0.05 mg to about 25 mg PDGF/cm², or about 0.1 mg to about 10 mg PDGF/cm², or about 0.2 mg to about 2 mg PDGF/cm². In some embodiments, each treatment with rhPDGF-AB is preferably between about 10 μg to 1000 μg PDGF/cm², or about 0.01 mg to about 50 mg PDGF/cm², or about 0.05 mg to about 25 mg PDGF/cm², or about 0.1 mg to about 10 mg PDGF/cm², or about 0.2 mg to about 2 mg PDGF/cm². In some embodiments, each treatment with rhPDGF-BB and rhPDGF-AB is preferably between about 10 μg to 1000 μg PDGF/cm², or about 0.01 mg to about 50 mg PDGF/cm², or about 0.05 mg to about 25 mg PDGF/cm², or about 0.1 mg to about 10 mg PDGF/cm², or about 0.2 mg to about 2 mg PDGF/cm². In some embodiments, each treatment applies an area dosage that is at least about 10 μg of rhPDGF/cm² of disruption surface area, or at least about 25 μg of rhPDGF/cm² of disruption surface area, or at least about 50 μg of rhPDGF/cm² of disruption surface area, or at least about 100 μg of rhPDGF/cm² of disruption surface area, or at least about 250 μg of rhPDGF/cm² of disruption surface area, or at least about 500 μg of rhPDGF/cm² of disruption surface area, or at least 1,000 μg of rhPDGF/cm² of disruption surface area. In some embodiments, each treatment applies an area dosage that is between about 100 μg of rhPDGF/cm² of disruption surface area and about 1,000 μg of rhPDGF/cm² of disruption surface area, or between about 300 μg of rhPDGF/cm² of disruption surface area and about 9,000 μg of rhPDGF/cm², or between about 400 μg of rhPDGF/cm² of disruption surface area and about 8,000 μg of rhPDGF/cm², or between about 10 μg of rhPDGF/cm² of disruption surface area and about 500 μg of rhPDGF/cm² of disruption surface area, or between about 10 μg of rhPDGF/cm² of disruption surface area and about 100 μg of rhPDGF/cm² of disruption surface area, or between about 15 μg of rhPDGF/cm² of disruption surface area and about 375 μg of rhPDGF/cm² of disruption surface area, or between about 30 μg of rhPDGF/cm² of disruption surface area and about 190 μg of rhPDGF/cm² of disruption surface area, or between about 30 μg of rhPDGF/cm² of disruption surface area and about 300 μg of rhPDGF/cm² of disruption surface area. In some embodiments, each treatment applies an area dosage that is between about 0.1 μg of rhPDGF/cm² of disruption surface area and about 10 μg of rhPDGF/cm² of disruption surface area. In some embodiments, each treatment applies an area dosage that is between about 0.2 μg of rhPDGF/cm² of disruption surface area and about 10 μg of rhPDGF/cm² of disruption surface area. In some embodiments, each treatment applies an area dosage that is between about 0.2 μg of rhPDGF/cm² of disruption surface area and about 1 μg of rhPDGF/cm² of disruption surface area. In some embodiments, each treatment applies an area dosage that is between about 0.5 μg of rhPDGF/cm² of disruption surface area and about 1 μg of rhPDGF/cm² of disruption surface area. In some embodiments, each treatment applies an area dosage that is between about 0.5 μg of rhPDGF/cm² of disruption surface area and about 10 μg of rhPDGF/cm² of disruption surface area. In some embodiments, each treatment applies an area dosage that is between about 1 μg of rhPDGF/cm² of disruption surface area and about 10 μg of rhPDGF/cm² of disruption surface area. In some embodiments, each treatment applies an area dosage that is between about 1 μg of rhPDGF/cm² of disruption surface area and about 5 μg of rhPDGF/cm² of disruption surface area. In some embodiments, each treatment applies an area dosage that is between about 2 μg of rhPDGF/cm² of disruption surface area and about 5 μg of rhPDGF/cm² of disruption surface area. In some embodiments, each treatment applies an area dosage that is between about 10 μg of rhPDGF/cm² of disruption surface area and about 100 μg of rhPDGF/cm² of disruption surface area. In some embodiments, each treatment applies an area dosage that is between about 100 μg of rhPDGF/cm² of disruption surface area and about 1,000 μg of rhPDGF/cm² of disruption surface area. In some embodiments, each treatment applies an area dosage that is between about 300 μg of rhPDGF/cm² of disruption surface area and about 1,000 μg of rhPDGF/cm² of disruption surface area.

In some embodiments, the initial treatment with compositions in accordance with the present invention may be the most important treatment. PDGF facilitates the disruption healing process through its effect on cell proliferation (mitogenesis) and directed cellular movement (chemotaxis) as well as re-vascularization (generating new blood vessels). PDGF can also improve cell survival by stimulating anti-apoptotic pathways. Many cells have been shown to possess receptors (binding sites) for PDGF including connective tissue cells (skin, bone, cartilage, tendon and ligament), blood vessel cells and cells of the nervous system. PDGF recruits and activates cells involved in mitogenesis such as stem cells, fibroblasts and smooth muscle cells. Cells that possess receptors for PDGF respond by migrating toward the site of the disruption (where PDGF is present at elevated levels as a result of applying therapeutic compositions in accordance with the present invention) and subsequently proliferating after binding PDGF. Since the PDGF receptor is degraded quickly after activation, cell proliferation is controlled and limited by the presence of locally available PDGF as well as by cell-cell interaction that leads cells to proceed from the proliferative phase of disruption healing to that of matrix deposition that ultimately results in complete healing. As a result, a critical bolus of rhPDGF-BB and/or rhPDGF-AB must be applied during the initial treatment to ensure that the patient's natural disruption healing process is properly activated. Therefore, in some embodiments, the initial treatment comprises applying a composition containing an area dosage that is at least 0.1 μg PDGF/cm² disruption surface area, up to 500 μg PDGF/cm² disruption surface area, at least 10 μg PDGF/cm² disruption surface area, up to 5000 μg PDGF/cm² disruption surface area, or at least 20 μg PDGF/cm² up to 1000 μg PDGF/cm² disruption surface area, or at least 30 μg PDGF/cm² up to 600 μg PDGF/cm² disruption surface area, or at least 40 μg PDGF/cm² up to 400 μg PDGF/cm² disruption surface area, or at least 50 μg PDGF/cm² up to 350 μg PDGF/cm² disruption surface area, or at least 60 μg PDGF/cm² up to 300 μg PDGF/cm² disruption surface area, or at least 200 μg PDGF/cm² up to 2000 μg PDGF/cm² disruption surface area. In some embodiments, the initial treatment comprises applying a therapeutic composition containing an area dosage that is at least 0.1 μg PDGF/cm² disruption surface area, at least 0.2 μg PDGF/cm² disruption surface area, at least 0.4 μg PDGF/cm² disruption surface area, at least 0.6 μg PDGF/cm² disruption surface area, at least 0.8 μg PDGF/cm² disruption surface area, at least 1 μg PDGF/cm² disruption surface area, at least 2 μg PDGF/cm² disruption surface area, at least 3 μg PDGF/cm² disruption surface area, at least 4 μg PDGF/cm² disruption surface area, at least 5 μg PDGF/cm² disruption surface area, at least 6 μg PDGF/cm² disruption surface area, at least 7 μg PDGF/cm² disruption surface area, at least 8 μg PDGF/cm² disruption surface area, at least 9 μg PDGF/cm² disruption surface area, at least 10 μg PDGF/cm² disruption surface area, or at least 20 μg PDGF/cm² disruption surface area, or at least 25 μg PDGF/cm² disruption surface area, or at least 30 μg PDGF/cm² disruption surface area, or at least 40 μg PDGF/cm² disruption surface area, or at least 50 μg PDGF/cm² disruption surface area, or at least 60 μg PDGF/cm² disruption surface area, or at least 70 μg PDGF/cm² disruption surface area, or at least 80 μg PDGF/cm² disruption surface area, or at least 90 μg PDGF/cm² disruption surface area, or at least 100 μg PDGF/cm² disruption surface area, or at least 250 μg PDGF/cm² disruption surface area, or at least 500 μg PDGF/cm² disruption surface area, or at least 600 μg PDGF/cm² disruption surface area, or at least 600 μg PDGF/cm² disruption surface area, or at least 700 μg PDGF/cm² disruption surface area, or at least or at least 800 μg PDGF/cm² disruption surface area, or at least 900 μg PDGF/cm² disruption surface area, or at least 1,000 μg PDGF/cm² disruption surface area, or at least 2,000 μg PDGF/cm² disruption surface area, or at least 5000 μg PDGF/cm² disruption surface area.

In some embodiments, each treatment applied is between about 4 μl PDGF solution/cm³ of carrier (which may be a hyaluronic acid gel or serum) to about 40 mL PDGF solution/cm³ of carrier, or between about 0.1 mL PDGF solution/cm³ of carrier to about 30 mL PDGF solution/cm³ of carrier, or between about 0.2 mL PDGF solution/cm³ of carrier to about 20 mL PDGF solution/cm³ of carrier, or between about 0.1 mL PDGF solution/cm³ of carrier to about 10 mL PDGF solution/cm³ of carrier, or between about 0.25 mL PDGF solution/cm³ of carrier to about 5 mL PDGF solution/cm³ of carrier, or between about 0.25 mL PDGF solution/cm³ of carrier to about 2.5 mL PDGF solution/cm³ of carrier, or between about 0.1 mL PDGF solution/cm³ of carrier to about 1 mL PDGF solution/cm³ of carrier, or between about 0.5 mL PDGF solution/cm³ of carrier to about 1.5 mL PDGF solution/cm³ of carrier. In certain embodiments, the PDGF solution contains about 0.3 mg/mL of rhPDGF-BB. In certain embodiments, the PDGF solution contains about 0.3 mg/mL of rhPDGF-AB. In certain embodiments, the PDGF solution contains about 0.3 mg/mL of rhPDGF-BB and rhPDGF-AB.

In some embodiments, each treatment applies between about 0.01 μg PDGF/cm³ of carrier to about 5 mg PDGF/cm³ of carrier, or between about 1.2 μg PDGF/cm³ of carrier to about 12 mg PDGF/cm³ of carrier, or between about 30 μg PDGF/cm³ of carrier to about 9 mg PDGF/cm³ of carrier, or between about 60 μg PDGF/cm³ of carrier to about 6 mg PDGF/cm³ of carrier, or between about 75 μg PDGF/cm³ of carrier to about 3 mg PDGF/cm³ of carrier, or between about 75 μg PDGF/cm³ of carrier to about 1.5 mg PDGF/cm³ of carrier, or between about 75 μg PDGF/cm³ of carrier to about 750 μg PDGF/cm³ of carrier, or between about 120 μg PDGF/cm³ of carrier to about 600 μg PDGF/cm³ of carrier, or between about 150 μg PDGF/cm³ of carrier to about 450 μg PDGF/cm³ of carrier, or between about 75 μg PDGF/cm³ of carrier to about 225 μg PDGF/cm³ of carrier.

In some embodiments, the initial PDGF treatment absolute dosage may be greater than the subsequent retreatment dosages. The initial PDGF treatment absolute dosage may be about 10%, about 20%, about 30%, about 40%, or about 50% higher, or up to about 300% higher than each of the subsequent retreatment PDGF dosages.

In some embodiments, the methods for treatment of acute skin disruptions as described herein may be similar to the methods for treatment of chronic wounds that are described in U.S. Pat. No. 10,071,182. However, the composition is applied to acute disruptions more frequently, but in fewer applications (e.g. 1-2 or 2-3 times day for up to 14 days). The methods for treating acute skin or connective tissue disruptions may also be similar to the methods used following micro-needling or other aesthetic procedures. In some embodiments described herein, the composition of the present invention used in the methods and uses described herein further comprises additional components, such as a local anesthetic. In other embodiments, the composition of the present invention used in the methods and uses described herein further comprise other PDGFs including compositions of PDGF-AA, PDGF-BB, PDGF-AB, PDGF-CC and/or PDGF-DD. In some embodiments described herein, the composition of the present invention used in the methods and uses described herein further compromises platelet-rich plasma (PRP), platelet-rich fibrin (PRF), and/or combinations thereof. However, PDGF has 1,000 to 300,000 times more growth factors than PRP, PRF or exosomes, so, in some embodiments, these additional components are optional.

In some embodiments of the methods and uses described herein, the carrier, such as hyaluronic acid, further comprises the aforementioned components (one or more of a local anesthetic) and is applied to the skin or other disruption separately from the composition before, during, and/or after the application procedure.

### EXAMPLES

The embodiments of the compositions, methods, uses and systems of the invention are described in more detail in the following non-limiting Examples.

### Example 1: Preparing a rhPDGF-BB Composition in a Hyaluronic Acid Carrier

This Example 1 describes a method for treating a skin disruption, such as hair loss, by applying a composition comprising rhPDGF-BB solution and hyaluronic acid, according to an embodiment. In the below described method, a composition of rhPDGF-BB solution with hyaluronic acid is used. However, a person skilled in the art would understand that any of the rhPDGF-BB and/or rhPDGF-AB-based compositions described herein may be used with the method described in Example 1.

In this exemplary method, a first step may include dispensing a volume (e.g., 3 mL) hyaluronic acid in a small dish. In one example, the hyaluronic acid may be in the form of, but not limited to, hyaluronic acid serum.

The next step may include dispensing a volume (e.g., 0.5 mL) of rhPDGF-BB solution into the dish containing the hyaluronic acid serum. In one embodiment, the rhPDGF solution may be composed of 300 ug/mL of rhPDGF-BB in 20 mM sodium acetate buffer having a pH value of 6.0+/−0.5 (i.e., 5.5-6.5). The dish, thus, now contains rhPDGF-BB solution and the hyaluronic acid serum in a 1:6 ratio.

Further, in this exemplary embodiment, the next step may include mixing the dispensed solution with the hyaluronic acid, thoroughly to form an rhPDGF-BB-based therapeutic composition with uniform consistency. The concentration of rhPDGF-BB in this exemplary therapeutic composition is 42.85 ug/mL. In an alternate embodiment, the composition may include a composition of rhPDGF-BB, hyaluronic acid and alginate. In yet another alternate embodiment, the composition may include a composition of rhPDGF-BB and a collagen or gelatin, wherein the collagen may be either soluble or insoluble. In yet another alternate embodiment, the rhPDGF-BB may be a powder, such as may be generated by freeze-drying or lyophilization, and the rhPDGF powder may be added to a carrier such as hyaluronic acid serum or a collagen, or collagen or gelatin or fibrin or other type of solution.

Example composition 1: An exemplary composition is prepared by mixing 0.5 mL of rhPDGF-BB solution at 300 ug/mL (300 ug/mL rhPDGF-BB in a 20 mM sodium acetate buffer) and 1.5 mL of a 1% hyaluronic acid (HA) gel or serum. The HA gel or serum is a blend of: (i) hyaluronic acid with a weight of between 100,000 Da and 150,000 Da and (ii) hyaluronic acid with a weight of between 750,000 Da and 900,000 Da at a 50:50 ratio. The resultant mixture contains 150 ug of PDGF in 2 mL of gel and has a final concentration of 75 ug PDGF/mL. The rhPDGF-BB solution and the hyaluronic acid serum are present in a 1:3 ratio. The resultant mixture contains 0.75 wt % of hyaluronic acid. Water may be added to the rhPDGF-BB therapeutic composition prior to applying the composition or may be present in the rhPDGF-BB solution, HA gel or serum, or both.

Example composition 2: A further exemplary composition is prepared by mixing 0.5 mL of rhPDGF-BB solution at 300 ug/mL (300 ug/mL rhPDGF-BB in a 20 mM sodium acetate buffer) and 3 mL of 1% hyaluronic acid (HA) gel or serum, which itself is a blend of (i) hyaluronic acid with a weight of between 100,000 Da and 150,000 Da and (ii) hyaluronic acid with a weight of between 750,000 Da and 900,000 Da. The resultant mixture contains 150 ug of PDGF in 3.5 mL of composition and has a final concentration of 42.86 μg PDGF/mL. The rhPDGF-BB solution and the hyaluronic acid serum are present in a 1:6 ratio. The resultant mixture contains 0.86 wt % of hyaluronic acid. Water may be added to the rhPDGF-BB therapeutic composition prior to applying the composition or may be present in the rhPDGF-BB solution, HA gel or serum, or both.

### Example 2: PDGF Promotes Hair Regrowth—Topical or Micro-Injection Treatment Following Tixel® Skin Perforation Treatment

This Example 2 describes methods for treating hair follicles and surrounding skin by performing the Tixel® skin perforation procedure, followed by applying a composition comprising rhPDGF-BB solution, according to an embodiment. In the below described method, a composition of rhPDGF-BB solution mixed with bacteriostatic saline is used. However, a person skilled in the art would understand that any of the rhPDGF-BB and/or rhPDGF-AB-based compositions described herein may be used with the methods described in Example 2.

After the Tixel® skin perforation procedure, approximately 2 mL of a PDGF composition (see the “Example Composition 1” in Example 1), was applied topically on and around the site of hair loss. After ten to fourteen days, 0.5 mL of rhPDGF-BB solution at 300 ug/mL (300 ug/mL rhPDGF-BB in a 20 mM sodium acetate buffer) mixed with 1.5 mL of bacteriostatic saline (2.0 mL total volume) was injected directly into the scalp using 5 prong 1.5 mL needles (32 gauge).

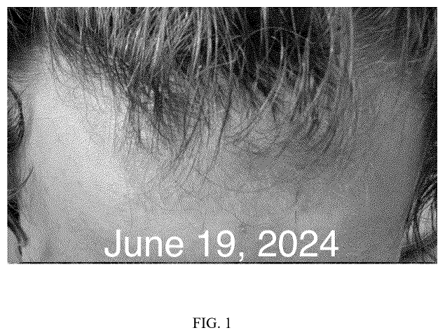

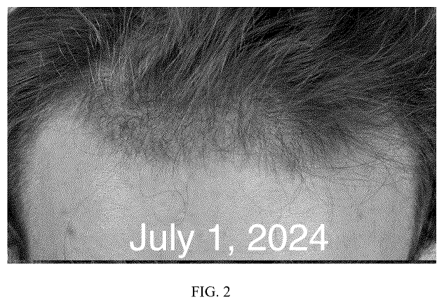

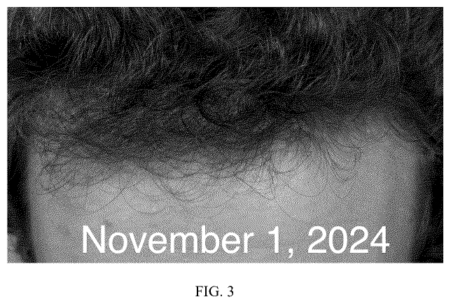

The patient took photographic images fourteen (14) days following the Tixel® skin procedure. By two weeks after the procedure and application, the hair growth was visually apparent (FIG. 2). Further, by 4 and a half months (19 weeks and 2 days), with a second treatment approximately 5 weeks after the first treatment, the patient demonstrated significant hair regrowth (FIG. 3). By comparison, FIG. 1 shows a photographic image taken prior to treatment with the PDGF composition, and after previous treatments following the standard of care, which involved a topical Minoxidil 7% with Finasteride 0.1% administered for two months and an oral Finasteride for one month and shows no hair regrowth.

### Example 3: PDGF Promotes Hair Regrowth—Micro-Injection Treatment

This Example 3 describes methods for treating hair follicles and surrounding skin by applying a therapeutic composition comprising rhPDGF-BB solution by micro-injection, according to an embodiment. In the below described method, a composition of rhPDGF-BB solution mixed with bacteriostatic saline is used. However, a person skilled in the art would understand that any of the rhPDGF-BB and/or rhPDGF-AB-based compositions described herein may be used with the methods described in Example 3.

Patients received approximately one to two units (i.e. syringes) of 0.5 mL of a PDGF composition (300 ug/mL rhPDGF-BB in a 20 mM sodium acetate buffer) diluted with 1.0 mL to 1.5 mL bacteriostatic saline applied via micro-injection to the scalp (1 cm apart) at the site of hair loss. Thus, the final dosing was 150 ug to 300 ug of rhPDGF-BB in a final volume of 2 mL to 4 mL of physiologic buffer, respectively. After four to eight weeks, the treatment was repeated.

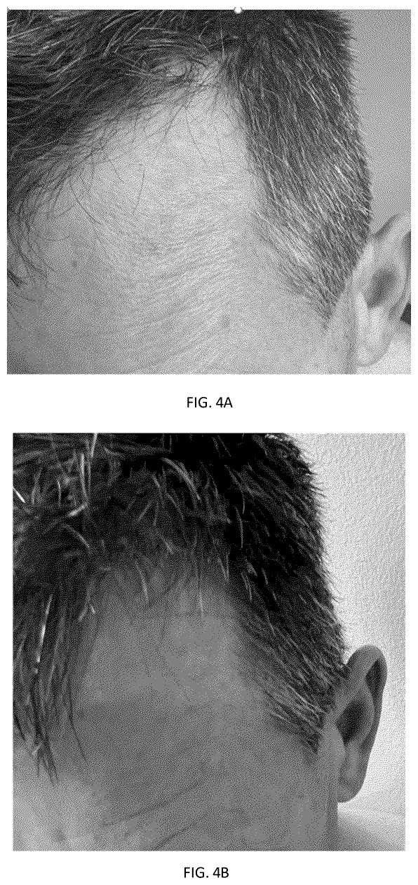

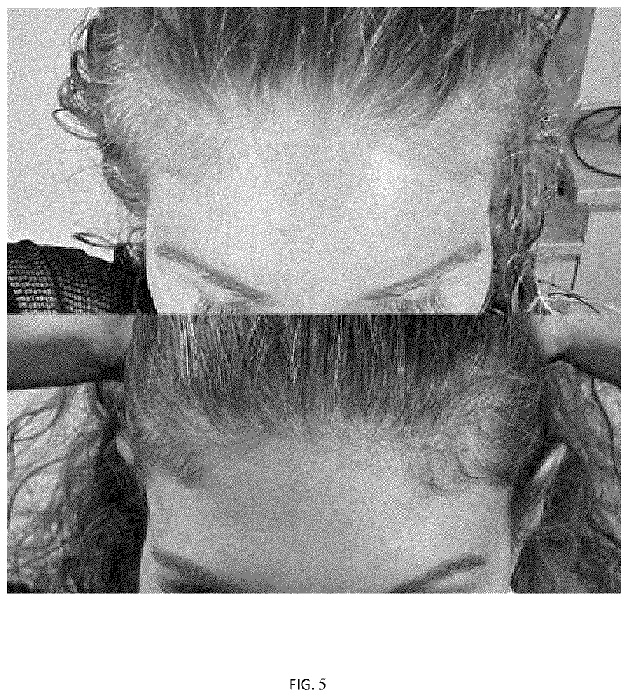

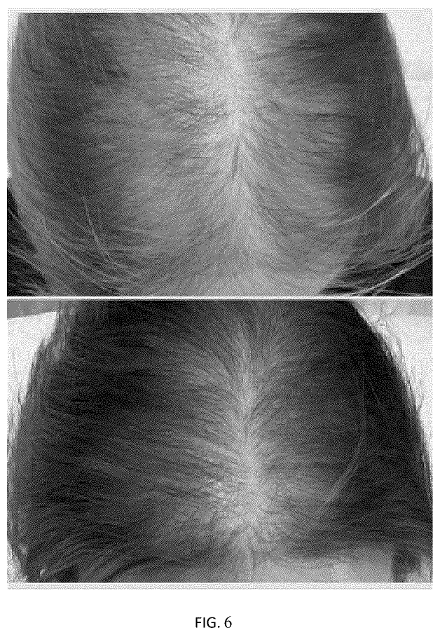

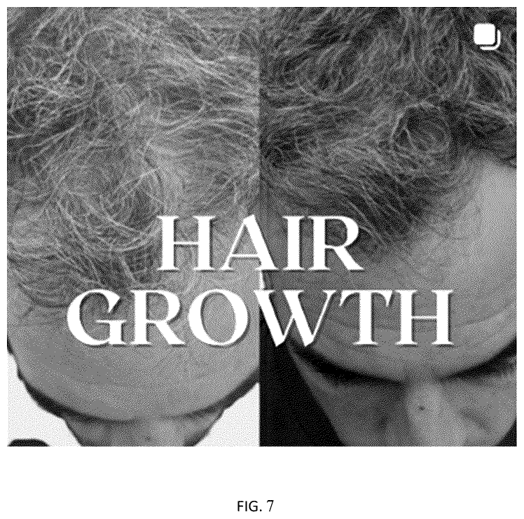

All patients demonstrated visually apparent hair regrowth in an image captured after the second treatment, in comparison to an image captured prior to treatment (FIGS. 4-7).

The invention has been described with reference to various specific and preferred embodiments and techniques. However, it should be understood, that many variations and modifications may be made while remaining within the spirit and scope of the invention. It will be apparent to one of ordinary skill in the art that methods, devices, device elements, materials, procedures, and techniques other than those specifically described herein can be applied to the practice of the invention as broadly disclosed herein without resort to undue experimentation. All art-known functional equivalents of methods, devices, device elements, materials, procedures, and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation. Additionally, it should be understood, that the various embodiments of the networks, devices, and/or modules described herein contain optional features that can be individually or together applied to any other embodiment shown or contemplated here to be mixed and matched with the features of such networks, devices, and/or modules.

While the invention has been described with respect to a limited number of embodiments, those skilled in the art, having benefit of this disclosure, will appreciate that other embodiments can be devised which do not depart from the scope of the invention as disclosed herein.

The embodiments, variations, and sequences described herein should provide an indication of the utility and versatility of the present invention. Other embodiments that do not provide all of the features and advantages set forth herein may also be utilized, without departing from the spirit and scope of the present invention. Such modifications and variations are considered to be within the scope of the invention.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process, when possible, as well as performed sequentially.

1.-63. (canceled)

64. A method of improving the pain or discomfort caused by a skin disruption on a subject, wherein the disruption does not involve tendons, ligaments, or bones, wherein said method comprises:
(1) providing a composition comprising sterile recombinant human PDGF-BB (rhPDGF-BB) and/or sterile recombinant human PDGF-AB (rhPDGF-AB) in a physiologic solution; and
(2) applying the composition onto or into the skin disruption leading to reduced pain or discomfort caused by the skin disruption.

65. The method of claim 64, wherein the composition does not comprise any additional growth factor, optionally wherein the composition does not comprise transforming growth factor (e.g. TGF beta), vascular endothelial growth factor (e.g. VEGF), keratinocyte growth factor (e.g. KGF), or connective tissue growth factor (e.g. CTGF).

66. The method of claim 64, wherein the composition further comprises a sterile biocompatible carrier, wherein the biocompatible carrier is a hyaluronic acid, collagen, gelatin, alginate, polysaccharide, glycosaminoglycan, fibrin, a derivative thereof or a combination thereof.

67. (canceled)

68. The method of claim 66, wherein the hyaluronic acid is a serum or gel.

69. The method of claim 66, wherein the composition comprises rhPDGF-BB and hyaluronic acid in a 1:3, 1:4, 1:5 or 1:6 ratio.

70. The method of claim 66, wherein the hyaluronic acid is a 50:50 blend of two molecular weights: (i) hyaluronic acid with a weight of between about 100,000 Da and 300,000 Da and (ii) hyaluronic acid with a weight of between about 700,000 Da and 950,000 Da; optionally wherein the hyaluronic acid is a 50:50 blend of (i) hyaluronic acid with a weight of between about 100,000 Da and 150,000 Da and (ii) hyaluronic acid with a weight of between about 750,000 Da and 900,000 Da.

71. The method of claim 64, wherein the skin disruption is caused by aging, inflammation, radiation damage, ultraviolet damage, or combinations thereof, optionally wherein the skin disruption comprises rough, dry or itchy skin that causes discomfort, or wherein the skin disruption is sunburn.

72. The method of claim 64, wherein the skin disruption is caused by an incision from a surgical procedure, such as an incision following facelift surgery; or a scar, such as a surgical scar.

73. The method of claim 64, wherein the skin disruption is caused by a skin penetrating aesthetic procedure, such as micro-needling, micro-injection, micro-coring, dermal abrasion, dermaplaning, dermal filling or laser skin resurfacing.

74. The method of claim 64, wherein the skin disruption extends only into the dermal or subdermal layers of the subject's skin.

75. The method of claim 64, further comprising monitoring the pain or discomfort of the skin disruption during a treatment period and repeating step (2) to retreat the skin disruption.

76.-77. (canceled)

78. The method of claim 64, wherein the method further comprises forming the composition by placing sterile recombinant human PDGF-BB (rhPDGF-BB) and/or sterile recombinant human PDGF-AB (rhPDGF-AB) in a physiologic solution, optionally wherein the method further comprises combining the sterile recombinant human PDGF-BB (rhPDGF-BB) and/or sterile recombinant human PDGF-AB (rhPDGF-AB) in a physiologic solution and a sterile biocompatible carrier, wherein the biocompatible carrier is a hyaluronic acid, collagen, gelatin, alginate, polysaccharide, glycosaminoglycan, fibrin, a derivative thereof or a combination thereof.

79. The method of claim 64, wherein the composition comprises an rhPDGF-BB solution comprising less than about 90 μg/g of rhPDGF-BB.

80. The method of claim 79, wherein the composition comprises an rhPDGF-BB solution comprising less than about 800 μg/g of rhPDGF-BB.

81.-86. (canceled)

87. The method of claim 64, wherein the composition comprises an rhPDGF-BB solution comprising less than about 90 μg/mL of rhPDGF-BB.

88. The method of claim 87, wherein the composition comprises an rhPDGF-BB solution comprising less than about 80 μg/mL of rhPDGF-BB.

89.-94. (canceled)

95. The method of claim 64, wherein the sterile rhPDGF-BB is at a final concentration of between about 200 μg rhPDGF-BB/mL of the composition and 400 μg rhPDGF-BB/mL of the composition; optionally wherein the sterile rhPDGF-BB is at a final concentration of about 300 μg rhPDGF-BB/mL of the composition.

96. The method of claim 64, wherein the sterile rhPDGF-BB is at a final concentration of between about 1.0 μg rhPDGF-BB/mL of the composition and 80 μg rhPDGF-BB/mL of the composition, optionally wherein the sterile rhPDGF-BB is at a final concentration of between about 40 μg rhPDGF-BB/mL of the composition and 80 μg rhPDGF-BB/mL of the composition.

97. (canceled)

98. The method of claim 64, wherein the composition comprises or consists of:
rhPDGF-BB at a final concentration of between about 200 μg/mL and 400 μg/mL; about 10 mM to about 30 mM sodium acetate; preferably about 20 mM sodium acetate; and water

99. The method of claim 64, wherein the composition is applied topically to the skin disruption.

100. The method of claim 64, wherein the composition is applied to the skin disruption before, during and/or after an aesthetic procedure is performed to penetrate the subject's skin or skin disruption e.g. using a mechanical device, a laser or a scraping tool; such as before, during and/or after a micro-needling, micro-injection, micro-coring, dermal abrasion, dermaplaning, dermal filling or laser skin resurfacing procedure is performed.

101.-102. (canceled)

103. The method of claim 100, wherein the mechanical device, laser or scraping tool penetrates the skin disruption at a depth between about 0.5 mm and 3 mm.

104.-105. (canceled)

106. The method of claim 100, wherein the penetration of the skin disruption is repeated optionally wherein the composition is re-applied between the repeat penetrations.

107. (canceled)

108. The method of claim 64, wherein the composition is applied to the skin disruption to achieve one or more of the following effects: improving appearance or aesthetics of the subject's skin, improving function of the subject's skin, stimulating cell growth, stimulating angiogenesis, improving texture of the subject's skin, for example by reducing wrinkles, furrows and/or sagging, enhance tissue volume, for example by reducing wrinkles, furrows and/or sagging, or prevent or reduce scarring of the subject's skin.

109.-114. (canceled)

115. The method of claim 64, wherein the composition is applied to the skin disruption to reduce redness, swelling, and/or inflammation, optionally to prevent or reduce redness, swelling and/or inflammation of a surgical incision, a scar, or the site at which an aesthetic procedure has been or will be applied, such as micro-needling, micro-injection, micro-coring, dermal abrasion, dermaplaning, dermal filling or laser skin resurfacing.

116. The method of claim 64, wherein the composition is applied to the skin disruption to regenerate, rejuvenate, or repair the skin disruption.

117. (canceled)

118. The method of claim 64, wherein the composition is applied into or onto the skin disruption via one or more needles; optionally wherein the composition is mixed with a dermal filler and applied by injection into or onto the skin disruption.

119. The method of claim 64, wherein the composition comprises an rhPDGF-BB solution comprising less than about 1,000 μg/mL of rhPDGF-BB.

120.-124. (canceled)

125. The method of claim 64, wherein the composition comprises an rhPDGF-BB solution comprising less than about 400 μg/mL of rhPDGF-BB

126.-127. (canceled)

128. The method of claim 64, wherein the composition is applied to the skin disruption to stimulate hair growth.

129. The method of claim 64, wherein the composition is applied to the skin disruption to treat hair loss.

130. The method of claim 64, wherein the composition comprises sterile recombinant human PDGF-BB (rhPDGF-BB) in a physiologic solution.

131. The method of claim 64, wherein the composition comprises sterile recombinant human PDGF-AB (rhPDGF-AB) in a physiologic solution.

132. The method of claim 64, wherein the composition comprises sterile recombinant human PDGF-BB (rhPDGF-BB) and sterile recombinant human PDGF-AB (rhPDGF-AB) in a physiologic solution.

133. A cosmetic method of promoting hair follicle regeneration and/or hair growth in a subject, wherein said method comprises:
(i) providing a composition comprising or consisting essentially of recombinant human PDGF-BB (rhPDGF-BB) and/or recombinant human PDGF-AB (rhPDGF-AB) in a physiologic solution; and
(ii) applying the composition onto or into the hair follicles and surrounding skin and/or area of hair loss.

134. The method of claim 133, wherein the composition comprises recombinant human PDGF-BB (rhPDGF-BB) in a physiologic solution.

135. The method of claim 133, wherein the composition comprises recombinant human PDGF-AB (rhPDGF-AB) in a physiologic solution.

136. The method of claim 133, wherein the composition comprises recombinant human PDGF-BB (rhPDGF-BB) and recombinant human PDGF-AB (rhPDGF-AB) in a physiologic solution.

137. The method of claim 133, wherein the rhPDGF-BB and/or rhPDGF-AB are sterile.

138. The method of claim 133, wherein the composition does not comprise any additional growth factor, optionally wherein the composition does not comprise transforming growth factor (e.g. TGF beta), vascular endothelial growth factor (e.g. VEGF), keratinocyte growth factor (e.g. KGF), or connective tissue growth factor (e.g. CTGF).

139. The method of claim 133, wherein the composition further comprises a biocompatible carrier, wherein the biocompatible carrier is a hyaluronic acid, collagen, gelatin, alginate, polysaccharide, glycosaminoglycan, fibrin, a derivative thereof or a combination thereof.

140.-141. (canceled)

142. The method of claim 139, wherein the composition comprises rhPDGF-BB and hyaluronic acid in a 1:3, 1:4, 1:5 or 1:6 ratio.

143. The method of claim 139, wherein the hyaluronic acid is a 50:50 blend of two molecular weights: (i) hyaluronic acid with a weight of between about 100,000 Da and 300,000 Da and (ii) hyaluronic acid with a weight of between about 700,000 Da and 950,000 Da; optionally wherein the hyaluronic acid is a 50:50 blend of (i) hyaluronic acid with a weight of between about 100,000 Da and 150,000 Da and (ii) hyaluronic acid with a weight of between about 750,000 Da and 900,000 Da.

144. The method of claim 133, wherein the area of hair loss is caused by aging, inflammation, radiation damage, ultraviolet damage, or combinations thereof.

145. The method of claim 133, wherein the area of hair loss is caused by an incision from a surgical procedure, such as an incision following facelift surgery; or a scar, such as a surgical scar.

146. The method of claim 133, wherein the area of hair loss is caused by a skin penetrating aesthetic procedure, such as micro-needling, micro-injection, micro-coring, dermal abrasion, dermaplaning, dermal filling or laser skin resurfacing.

147. The method of claim 133, wherein the hair follicles and surrounding skin are on a subject having reduced total hair count, hair density, mean thickness, and/or cumulative hair thickness, in comparison to measurements taken from the subject at a previous time or average measurements.

148. The method of claim 147, wherein the reduced total hair count, hair density, mean thickness, and/or cumulative hair thickness is caused by aging, inflammation, radiation damage, ultraviolet damage, or combinations thereof.

149. The method of claim 147, wherein the reduced total hair count, hair density, mean thickness, and/or cumulative hair thickness is caused by an incision from a surgical procedure or other medical intervention, such as chemotherapy, radiation, or medication.

150. The method of claim 147, wherein the reduced total hair count, hair density, mean thickness, and/or cumulative hair thickness is caused by scarring or an aesthetic procedure, such as micro-needling, micro-injection, micro-coring, dermaplaning, dermal abrasion, dermal filling or laser resurfacing.

151. The method of claim 133, further comprising monitoring the hair follicle regeneration and/or hair growth during a treatment period and repeating step (ii) of the method to retreat the hair follicles and surrounding skin and/or area of hair loss.

152.-153. (canceled)

154. The method of claim 133, wherein the method further comprises forming the composition by placing rhPDGF-BB and/or rhPDGF-AB in a physiologic solution, optionally wherein the method further comprises combining the rhPDGF-BB and/or rhPDGF-AB in a physiologic solution and a biocompatible carrier, wherein the biocompatible carrier is a hyaluronic acid, collagen, gelatin, alginate, polysaccharide, glycosaminoglycan, fibrin, a derivative thereof or a combination thereof.

155. The method of claim 133, wherein the composition comprises an rhPDGF-BB solution comprising less than about 90 μg/g of rhPDGF-BB.

156. The method of claim 155, wherein the composition comprises an rhPDGF-BB solution comprising less than about 80 μg/g of rhPDGF-BB.

157.-162. (canceled)

163. The method of claim 133, wherein the composition comprises an rhPDGF-BB solution comprising less than about 90 μg/mL of rhPDGF-BB.

164. The method of claim 163, wherein the composition comprises an rhPDGF-BB solution comprising less than about 80 μg/mL of rhPDGF-BB.

165.-170. (canceled)

171. The method of claim 133, wherein the rhPDGF-BB is at a final concentration of between about 200 μg rhPDGF-BB/mL of the composition and 400 μg rhPDGF-BB/mL of the composition; optionally wherein the rhPDGF-BB is at a final concentration of about 300 μg rhPDGF-BB/mL of the composition

172. The method of claim 133, wherein the rhPDGF-BB is at a final concentration of between about 1.0 μg rhPDGF-BB/mL of the composition and 80 μg rhPDGF-BB/mL of the composition, optionally wherein the rhPDGF-BB is at a final concentration of between about 40 μg rhPDGF-BB/mL of the composition and 80 μg rhPDGF-BB/mL of the composition.

173. (canceled)

174. The method of claim 133, wherein the composition comprises or consists of:
rhPDGF-BB at a final concentration of between about 200 μg/mL and 400 μg/mL; about 10 mM to about 30 mM sodium acetate; preferably about 20 mM sodium acetate; and water.

175. The method of claim 133, wherein the composition is applied topically to the hair follicle and surrounding skin and/or area of hair loss.

176. The method of claim 133, wherein the composition is applied to the hair follicle and surrounding skin and/or area of hair loss before, during and/or after an aesthetic procedure is performed to penetrate the subject's hair follicle and surrounding skin and/or area of hair loss e.g. using a mechanical device, a laser or a scraping tool; such as before, during and/or after a micro-needling, micro-injection, micro-coring, dermal abrasion, dermaplaning, dermal filling or laser skin resurfacing procedure is performed.

177.-178. (canceled)

179. The method of claim 176, wherein the mechanical device, laser or scraping tool penetrates the skin disruption at a depth between about 0.5 mm and 3 mm.

180.-181. (canceled)

182. The method of claim 176, wherein the penetration of the skin disruption is repeated optionally wherein the composition is re-applied between the repeat penetrations.

183. (canceled)

184. The method of claim 133, wherein the composition is applied to the hair follicle and surrounding skin and/or area of hair loss to improve appearance or aesthetics of the subject's hair.

185. (canceled)

186. The method of claim 133, wherein the composition is applied into or onto the hair follicle and surrounding skin and/or area of hair loss via one or more needles.

187.-188. (canceled)
